(19) United States
(12) Patent Application Publication
(10) Pub. No.: US 2025/0090658 A1
(43) Pub. Date: Mar. 20, 2025

(54) POLYMER ADJUVANT

(71) Applicants: Oxford University Innovation Limited, Oxford (GB); The United States of America, as represented by the Secretary, Department of Health and Human Servic, Bethesda, MD (US)

(72) Inventors: Kerry Fisher, Oxford (GB); Richard Laga, Prague 6 (CZ); Geoffrey Lynn, Bethesda, MD (US); Leonard Seymour, Oxford (GB); Robert Seder, Bethesda, MD (US)

(73) Assignees: Oxford University Innovation Limited, Oxford (GB); The United States of America, as represented by the Secretary, Department of Health and Human Servic, Bethesda, MD (US)

(21) Appl. No.: 18/970,451

(22) Filed: Dec. 5, 2024

Related U.S. Application Data
(62) Division of application No. 16/907,912, filed on Jun. 22, 2020.
Application No. 16/907,912 is a continuation of application No. 15/517,121, filed on Apr. 5, 2017, now abandoned.

(30) Foreign Application Priority Data
Oct. 10, 2014 (GB) 1418004.6

(51) Int. Cl. A61K 39/39; A61K 39/00; A61K 39/12; A61K 39/385; A61K 47/58; A61K 47/60; C08F 220/54; C08F 220/56; C08F 220/60

(52) U.S. Cl. CPC A61K 39/39; A61K 39/0011; A61K 39/12; A61K 39/385; A61K 47/58; A61K 47/60; C08F 220/56; A61K 2039/55511; A61K 2039/55516; A61K 2039/55555; A61K 2039/55561; A61K 2039/55572; A61K 2039/6093; A61K 2039/627; C08F 220/54; C08F 220/606; C12N 2740/16234

(57) ABSTRACT

The invention relates to an adjuvant comprising Pattern Recognition Receptor (PRR) agonist molecules linked to polymer chains that are capable of undergoing particle formation in aqueous conditions, or in aqueous conditions in response to external stimuli; and methods of treatment or prevention of disease using such an adjuvant.

### CROSS-REFERENCE TO RELATED APPLICATIONS

This application is a divisional of co-pending U.S. application Ser. No. 16/907,912 filed Jun. 22, 2020, which is a continuation of U.S. application Ser. No. 15/517,121 filed Apr. 5, 2017, which is a 35 U.S.C. § 371 National Phase Entry Application of International Application No. PCT/GB2015/05/052974 filed Oct. 9, 2015, which claims benefit of, and priority to, GB Application No. 1418004.6 filed Oct. 10, 2014, the contents of each of which are incorporated herein by reference in their entireties.

### SEQUENCE LISTING

The instant application contains a Sequence Listing which has been submitted electronically in XML format and is hereby incorporated by reference in its entirety. Said XML file, created on Dec. 4, 2024, is named BARI-002.xml and is 5.1 kilobytes in size.

This invention relates to an adjuvant, an immunogenic composition, and manufacture of such adjuvants and compositions, and their use.

Vaccines that elicit potent and durable cellular immunity (CD4 and CD8 T-cells) are needed for protection against certain infections (e.g. malaria and tuberculosis) or as therapies for cancer.

While there are several vaccine platforms (whole organism, viral vectors, etc.) for inducing T cell responses, many efforts are focused on protein-based vaccines, which are safe, scalable and capable of being used repetitively to boost immunity. A limitation is that protein is weakly immunogenic when administered alone and requires the addition of adjuvants, such as pattern recognition receptor agonists (PRRa), that improve cellular immune responses primarily through activation of antigen presenting cells (APCs) that provide the signals required for priming, differentiating and expanding T cells.

Adjuvants are often used to improve and refine the immune response to an antigen. Accounting for the delivery of certain adjuvants, particularly molecularly defined, PRRa, which includes Toll-like receptor agonists (TLRa), is critical for optimizing their in vivo activity for use with protein antigens. For instance, formulating or delivering TLRa in or on particles mixed with protein antigen, or even attaching TLRa directly to antigen, have all been shown to markedly improve CD4 and CD8 T cell responses. Improved responses likely arise from the combined affects that these formulation and delivery approaches have on TLRa pharmacokinetics and APC uptake, whereby increased local retention of a particulate carrier could prolong the persistence of innate immune (e.g., APC) activation in lymph nodes that is important for T cell priming.

An aim of the present invention is to provide an improved adjuvant for use in eliciting an immune response in a subject.

According to a first aspect of the invention, there is provided an adjuvant comprising PRRa molecules linked to polymer chains that are capable of undergoing particle formation in aqueous conditions, or in aqueous conditions in response to external stimuli; and optionally wherein the polymer is a unimolecular polymer chain.

In one embodiment, the polymer is a linear or branched polymer, such as a linear or branched unimolecular polymer chain.

In one embodiment, the polymer is a thermo-responsive polymer.

Advantageously, the invention can be used to provide a persistent innate immune activation. In particular, the invention advantageously provides a particle-forming adjuvant, or pre-formed particle adjuvant, that can enhance innate immune activation in lymph nodes by increasing local retention and promoting uptake by APCs (antigen presenting cells). Linking PRR agonist molecules to unimolecular polymer chains with thermo-responsive properties enables particle formation after administration, providing advantages in manufacturing and storage over the use of preformed particles with or without thermo-responsive properties. For example, sterile filtration is the most cost-effective means of purifying solutions used for vaccines and typically requires that all the components are smaller than about 200 nm. This requirement precludes the use of many pre-formed particle based vaccines that are larger than this size, or it requires more expensive and labour-intensive purification strategies. By using thermo-responsive polymers that exist as single unimolecular chains that are, for example, ˜10-20 nm in diameter in aqueous conditions, sterile-filtration can be used and still have the capability to form any desired size particles in vivo. For storage, particles tend to aggregate over time in solution, reducing the chemical definition (e.g., increases variability and decreases reproducibility) and even the concentration of the active molecules (e.g., if the particles aggregate and become insoluble). It's therefore advantageous to have a means of storing the composition with reduced potential that any particles may aggregate over time.

A further advantage of the ability to form particles after administration is that local tissue damage may be minimised, and it can be potentially less painful for a subject to receive an administration of a non-particulate solution relative to a pre-formed particulate. Higher density of PRR agonists clustered on the formed particles is also achievable for particles formed in situ relative to pre-formed particles where density of the PRR agonist is limited by steric hindrance. Advantageously, the in situ formation of particles may allow the formation of a more heterogeneous mixture of particle sizes that can provide a more favourable immune response relative to more uniform pre-fabricated particles.

The term “pre-formed” in relation to particles is understood to mean that the particles are provided/formed prior to any administration of the adjuvant to a subject, and they do not substantially form post-administration in situ. For example, the particles may be formed during manufacture or preparation of the adjuvant from linear or branched unimolecular polymer chains with linked PRRa. That the particles are formed from PRRa linked to linear or branched unimolecular polymer chains provides the advantages in terms of manufacturing and storage as compared with particles that are fabricated first and then linked to PRRa. For instance, a higher density of PRRa per particle can be obtained by inducing the polymers linked to PRRa to form particles, rather than reacting PRRa with pre-formed particles.

The terms “linear or branched polymers” may also be referred to as “unimolecular polymer chains”, and it is intended that such terms may be used interchangeably.

The term “in aqueous conditions” in the context of linear or branched polymers is understood to mean that the linear or branched polymer is in solution or a suspension.

The external stimuli may comprise a change in temperature/a temperature shift. The temperature shift may be an increase in temperature. The external stimuli may comprise a change in pH. The change in pH may be an increase in acidity, a decrease in pH. The change in pH may be an increase in alkalinity, an increase in pH. The pH shift may be a result of a natural physiological process, such as the acidification of an intracellular vesicle from pH 7.4 to pH 5.5. The pH shift may be a result of high metabolic activity at the site of an inflamed tissue, which can result in glycolis and production of acidic substrates. The pH shift may be a result of a cancer that creates an acidic microenvironment due to high rates of glycolysis, which may result in production of an acidic substrate (Warburg effect).

In one embodiment, the adjuvant comprised of PRRa linked to unimolecular polymer chains may be capable of assembling into particles in response to a temperature shift, for example where thermo-responsive polymer is used. In another embodiment, the adjuvant may comprise PRRa linked to unimolecular polymer chains that assemble into particles in aqueous conditions due to the hydrophobic nature of attached ligand molecules (pre-formed polymer particles). Therefore, in one embodiment, the polymers, such as linear or branched unimolecular polymer chains, may be capable of undergoing particle formation in aqueous conditions (for example in the absence of temperature change stimulus). In another embodiment, the polymers may be thermo-responsive and are capable of undergoing particle formation in response to a temperature shift.

The term “particle formation” is understood to mean assembly of multiple linear or branched unimolecular (single molecule) polymer chains into higher order structures, including micelles, nano-sized supramolecular associates and/or submicron to micron-sized particles. The particles (either pre-formed, or formed after a temperature shift) may be a size capable of being phagocytosed, for example from about 2 to about 5,000 nm in size. Alternatively, larger particles may be formed or provided, that allow slow release of smaller particles, the agonist, and/or the antigen. The adjuvant may be, or may be capable of assembling into, particles of defined sizes of between about 20 nm and about 10,000 nm. The adjuvant may be, or may be capable of assembling into, particles of defined sizes of between about 20 nm and about 5,000 nm. The adjuvant may be, or may be capable of assembling into, particles of defined sizes of between about 20 nm and about 1,000 nm. The adjuvant may be, or may be capable of assembling into, particles of defined sizes of between about 20 nm and about 100 nm. The adjuvant may be, or may be capable of assembling into, particles of defined sizes of between about 25 nm and about 100 nm. The adjuvant may be, or may be capable of assembling into, particles of defined sizes of between about 30 nm and about 100 nm. The adjuvant may be, or may be capable of assembling into, particles of defined sizes of between about 20 nm and about 99 nm. The adjuvant may be, or may be capable of assembling into, particles of defined sizes of between about 30 nm and about 99 nm. The adjuvant may be, or may be capable of assembling into, particles of defined sizes of between about 20 nm and about 95 nm. The adjuvant may be, or may be capable of assembling into, particles of defined sizes of between about 30 nm and about 95 nm. The adjuvant may be, or may be capable of assembling into, particles of defined sizes of between about 20 nm and about 90 nm. The adjuvant may be, or may be capable of assembling into, particles of defined sizes of between about 30 nm and about 90 nm. The adjuvant may be, or may be capable of assembling into, particles of defined sizes of between about 500 nm and about 8,000 nm. The adjuvant may be, or may be capable of assembling into, particles of defined sizes of between about 100 nm and about 2,000 nm. The adjuvant may be, or may be capable of assembling into, particles of defined sizes of between about 20 nm and about 200 nm. The adjuvant may be, or may be capable of assembling into, particles of defined sizes of between about 50 nm and about 400 nm. The adjuvant may be, or may be capable of assembling into, particles of defined sizes of between about 50 nm and about 200 nm. The adjuvant may be, or may be capable of assembling into, particles of defined sizes of between about 50 nm and about 100 nm. The adjuvant may be, or may be capable of assembling into, particles of defined sizes of between about 30 nm and about 110 nm. The adjuvant may be, or may be capable of assembling into, particles of defined sizes of between about 40 nm and about 105 nm. The adjuvant may be, or may be capable of assembling into, particles of defined sizes of less than about 100 nm. The adjuvant may be, or may be capable of assembling into, particles of defined sizes of less than about 10,000 nm. The adjuvant may be, or may be capable of assembling into, particles of defined sizes of less than about 1,000 nm. The adjuvant may be, or may be capable of assembling into particles of defined sizes of less than about 500 nm. The adjuvant may be, or may be capable of assembling into particles of defined sizes of greater than about 20 nm. The adjuvant may be, or may be capable of assembling into particles of defined sizes of greater than about 50 nm. The adjuvant may be, or may be capable of assembling into particles of defined sizes of greater than about 100 nm. The assembly may be in response to a temperature shift in embodiments requiring thermo-responsive polymer. In one embodiment, the size of the particles may be the average size of their longest dimension within a population of particles. In another embodiment, all of the particles in a population may be within the defined size, as measured by the longest dimension of the particle.

The adjuvant of the invention may be for local administration to a specific tissue, site, or region of the body. The adjuvant of the invention may be substantially retained in the body at the site of administration, for example at least 95% of the adjuvant may be retained at the site of administration. In another embodiment, at least 90%, 80% or 70% of the adjuvant may be retained at the site of administration. The adjuvant may be retained locally and persist in draining lymph nodes for at least 5 days. The adjuvant may be retained locally and persist in draining lymph nodes for at least 10 days. The adjuvant may be retained locally and persist in draining lymph nodes for at least 15 days. The adjuvant may be retained locally and persist in draining lymph nodes for at least 18 days. The term “retained” means that the adjuvant does not become substantively dispersed or systemic after local administration. Reference to “does not become substantially systemic” is understood to be an asymmetric pattern of biodistribution wherein local concentrations of a drug are higher than concentrations in systemic circulation following non-systemic routes of administration.

There are a variety of scaffolds with thermo-responsive properties that are suitable for the delivery of immune potentiators (e.g., pattern recognition receptor (PRR) agonists). A feature of thermo-responsive polymeric scaffolds is that the materials undergo temperature dependent conformational changes that minimize the polymer-solvent contacts and maximize contacts between monomers, a process that results in the polymers scaffolds undergoing transition from a random coil to a collapsed globular, or micellar, structure in aqueous conditions, resulting in multiple polymer chains coming together to form multimeric particles. The thermo-responsive polymer may exhibit a lower critical solution temperature (LCST)-type phase diagram, where the critical temperature Tc indicating the coil-globule transition of the macromolecular chain is ≤40° C. in aqueous solutions. The thermo-responsive polymer may exhibit a lower critical solution temperature (LCST)-type phase diagram, where the critical temperature Tₑ indicating the coil-globule transition of the macromolecular chains is ≤37° C. in aqueous solutions. The thermo-responsive polymer may be responsive to a temperature shift from below body temperature (for example less than about 36° C.) to body temperature (about 37° C.) or more. In an alternative embodiment, the thermo-responsive polymer may be responsive to a temperature shift from below 39° C. to a temperature of about 40° C. or more. The thermo-responsive polymer may conformation may change from a random coil to a collapsed globular or a micellar shape depending on temperature changes of the environment to minimize the polymer-solvent contacts and maximize the contacts between monomers. The thermo-responsive polymer may have a lower critical solution temperature (LCST) (otherwise referred to as the “phase transition temperature” or “coil-globule transition temperature”) of less than 36° C. The thermo-responsive polymer may have a lower critical solution temperature of less than 35° C. The thermo-responsive polymer may have a lower critical solution temperature of between about 4° C. and about 40° C. The thermo-responsive polymer may have a lower critical solution temperature of between about 4° C. and about 37° C. The thermo-responsive polymer may have a lower critical solution temperature of between about 4° C. and about 36° C. The thermo-responsive polymer may have a lower critical solution temperature of between about 20° C. and about 37° C. The thermo-responsive polymer may have a lower critical solution temperature of between about 20° C. and about 36° C. The thermo-responsive polymer may have a lower critical solution temperature of between about 20° C. and 35° C. The thermo-responsive polymer may have a lower critical solution temperature of between 24° C. and 36° C. The thermo-responsive polymer may have a lower critical solution temperature of between 30° C. and 35° C.

In one embodiment, the lower critical solution temperature may be higher than normal body temperature, for example 40° C., or more. The lower critical solution temperature may be higher than 37° C. The lower critical solution temperature may be between about 38° C. or 39° C. and 42° C. The adjuvant may be capable of forming particles at the site of radiation, for example during tumour therapy, where the local tissue is heated to a temperature above the surrounding tissue, for example above body temperature. The adjuvant may be capable of forming particles at the site of inflammation, for example during infection, where the local tissue is heated to a temperature above the surrounding tissue, for example above body temperature. Such a lower critical solution temperature would advantageously allow particles to be formed at specific tissue sites, such as in tumour tissue.

The linear or branched unimolecular polymers may exist as single unimolecular chains that are ˜1-20 nm in diameter in aqueous conditions. The linear or branched unimolecular polymers may exist as single unimolecular chains that are ˜1-20 nm in diameter in aqueous conditions and in the absence of external stimuli. In non-aqueous conditions the linear or branched unimolecular polymer chains may exist as single unimolecular chains that can adopt an extended coil conformation or globular morphology.

In embodiments wherein the polymer is not in aqueous conditions (i.e. in non-aqueous conditions) the polymer may be suspended or dissolved in organic solvents. Examples of organic solvents include methanol, DCM and DMSO, and the skilled person will be familiar with the range of organic solvents suitable as a carrier or solute for the polymer. The organic solvent may be a pharmaceutically acceptable organic solvent. In another embodiment, the non-aqueous conditions may refer to the adjuvant comprising the polymer being lyophilised, for example for storage. Upon reconstitution with water, the polymer may collapse to form the compact globuli/particle. Alternatively, upon reconstitution with water, the polymer may be arranged to remain as a unimolecular polymer dispersed in the water, and may only further collapse to form the compact globuli/particle in response to the external stimuli.

The polymer may collapse in solution to form the compact globuli/particle. In another embodiment, the thermo-responsive polymer chain in solution may have an extended coil conformation (e.g., about 10 nm in size, or in some embodiments about 5-20 nm in size), which will collapse to form a compact globuli/particle at the phase separation temperature of the thermo-responsive polymer. In alternative embodiments, where block- or graft-copolymers with amphiphilic character are used (e.g., where one block (or graft) is formed by thermo-responsive chains and the second one consist of hydrophilic chains), the macromolecules may collapse into micelles. The thermo-responsive polymer may be, or arranged to be, globular in structure at body temperature (e.g., at 37° C.). The thermo-responsive polymer may be, or arranged to be, extended-coil/non-globular in structure at room temperature (for example at 24° C.).

The lower critical solution temperature may be determined by turbidimetry. The lower critical solution temperature may be defined as the temperature at the onset of cloudiness, the temperature at the inflection point of the transmittance curve, or the temperature at a defined transmittance (e.g., 50%). The lower critical solution temperature may be calculated from the intersection point of two lines formed by linear regression of a lower horizontal asymptote and a vertical section of the sigmoidal curve (S-shaped curve).

A thermo-responsive polymer, such as pNIPAM (poly (NIPAM), may be modified by copolymerization with an appropriate monomer or with linking moieties and/or branches to alter the lower critical solution temperature to the required temperature. The lower critical solution temperature of any given polymer molecule may be influenced by incorporating molecules with different hydrophilic/hydrophobic characteristics. For example, agonist molecules based on highly hydrophobic Pam3Cys statistically attached along the backbone of a thermo-responsive polymer may be used to significantly decrease its lower critical solution temperature, while incorporation of hydrophilic CpG-based agonist will have the reverse effect.

The polymer may be biodegradable, for example biodegradable in the body. The polymer may be held together by bonds (for example, amide, esters, or the like) that can undergo hydrolysis in the body to release small molecules that can be eliminated through renal or hepatic excretion.

The polymer may be biocompatible. It is understood that the term “biocompatible” may comprise non-toxic to a human or animal body, for example at therapeutically relevant doses. The polymer may not be antigenic in the absence of any antigenic molecules linked thereto.

The polymer may be a homopolymer, a copolymer a block-copolymer or a graft copolymer. In one embodiment, the polymer is linear. In another embodiment the polymer is branched. In another embodiment, a mixture of linear and branched polymers may be provided.

The polymer may comprise or consist of monomers of any of the group selected from N-isopropylacrylamide (NIPAM); N-isopropylmethacrylamide (NIPMAM); N,N′-diethylacrylamide (DEAAM); N-(L)-(1-hydroxymethyl) propyl methacrylamide (HMPMAM); N,N′-dimethylethylmethacrylate (DMEMA), 2-(2-methoxyethoxy)ethyl methacrylate (DEGMA); pluronic, PLGA and poly(caprolactone); or combinations thereof. The polymer may comprise or consist of block-copolymer, such as NIPAM-HPMA or NIPAM-PLGA. The polymer may comprise or consist of graft-copolymers, for example NIPAM with protein or PLGA attached to side chains. The polymer may comprise HPMA (N-(2-Hydroxypropyl) methacrylamide). In embodiments where a specific thermo-responsiveness is not necessary, e.g. in pre-formed particles, other polymers may be considered, such as PLGA. Suitable pre-formed particles or non-thermoresponsive polymers may include those that are produced by chain growth polymerization using radical donating species to initiate polymerization of monomers having a vinyl moiety. Such polymers may comprise of monomers with (meth)acrylates, (meth)acrylamides, styryl and vinyl moieties. Specific examples of (meth)acrylates, (meth)acrylamides, as well as styryl- and vinyl-based monomers include N-2-hydroxypropylmethacrylamide (HPMA), hydroxyethylmethacrylate (HEMA), Styrene and vinylpyrrolidone (PVP), respectively. Non-thermo-responsive polymers or particles can also be based on cyclic monomers that include cyclic urethanes, cyclic ethers, cyclic amides, cyclic esters, cyclic anhydrides, cyclic sulfides and cyclic amines. Polymers based on cyclic monomers may be produced by ring opening polymerization and include polyesters, polyethers, polyamines, polycarbonates, polyamides, polyurethanes and polyphosphates; specific examples may include but are not limited to polycaprolactone and polyethylenimine (PEI). Suitable polymers may also be produced through condensation reactions and include polyamides, polyacetals and polyesters.

Non-thermoresponsive polymers may be based on biopolymers or naturally occurring monomers and combinations thereof. Natural biopolymers may include single or double stranded RNA or DNA, comprised of nucleotides (e.g., adenosine, thymidine). The natural biopolymers can be peptides comprised of amino acids; a specific example is poly(lysine). Biopolymers can be polysaccharides, which may include but is not limited to glycogen, cellulose and dextran. Additional examples include polysaccharides that occur in nature, including alginate and chitosan. Non-thermoresponsive polymers may also be comprised of naturally occurring small molecules, such as lactic acid or glycolic acid, or may be a copolymer of the two (i.e., PLGA). Suitable preformed particles may also be based on formulations (e.g., stabilized emulsions, liposomes and polymersomes) or may be mineral salts that form particles suitable for complexation or ion exchange on the surfaces of the particles, which may include Aluminum-based salts.

The average molecular weights of the polymer may be between about 5,000 to 1,000,000 g/mol. The polydispersity indexes of the polymer may range from about 1.1 to about 5.0.

The adjuvant composition may be suitable for, or capable of, eliciting an immune response in a mammal, such as a human. The immune response may comprise a protective immune response. The immune response may comprise an antibody response. The immune response may comprise a T-cell response. The T-cell response may comprise a CD4 and/or CD8 T-cell response. The T-cell response may comprise a CD8 T-cell response. The T-cell response may comprise a CD4 T-cell response. The immune response may comprise a TH₁ and/or TH₂ cell response. The immune response may comprise a TH₁ cell response. The immune response may comprise an antibody and T cell response.

The Pattern Recognition Receptor (PRR) agonist may comprise any of a broad and diverse class of synthetic or naturally occurring compounds that are recognized by pattern recognitions receptors (PRRs). The Pattern Recognition Receptor (PRR) agonist may comprise a PAMP (pathogen-associated molecular pattern). The PRR agonist may comprise a TLR agonist. The TLR agonist may comprise any TLR agonist selected from the group comprising TLR-1/2/6 agonists (e.g., lipopeptides and glycolipids, such as Pam2cys or Pam3cys lipopeptides); TLR3 agonists (e.g., dsRNA and nucleotide base analogs), TLR4 (e.g., lipopolysaccharide (LPS) and derivatives); TLR5 agonists (Flagellin); TLR-7/8 agonists (e.g., ssRNA and nucleotide base analogs); and TLR-9 agonists (e.g., unmethylated CpG); or combinations thereof. The TLR agonist may comprise a TLR-7/8 agonist. The TLR agonist may comprise an imidazoquinoline compound. The TLR agonist may comprise R848, or a functionally equivalent derivative or analogue thereof. The TLR agonist may comprise a more potent variant of R848. The more potent variant of R848 may be characterised by a more hydrophobic and planar linker instead of a flexible alkane chain. The TLR agonist may comprise a TLR-7 agonist such as Imiquimod (R837) or a functionally equivalent analogue thereof.

The PRR agonist may comprise NOD-like receptor (NLR) agonists, such as peptidogylcans and structural motifs from bacteria (e.g., meso-diaminopimelic acid and muramyl dipeptide). The PRR agonist may comprise agonists of C-type lectin receptors (CLRs), which include various mono, di, tri and polymeric sugars that can be linear or branched (e.g., mannose, Lewis-X tri-saccharides, etc.). The PRR agonist may comprise agonists of STING (stimulator of interferon [IFN] genes) (e.g., cyclic dinucleotides, such as cyclic diadenylate monophosphate).

The adjuvant composition may be combined with a suitable antigen to create an immunogenic composition that can be administered to a subject. The antigen may be a protein or peptide antigen or a poly(saccharide) derived from a pathogen or tumor. The antigen may be co-administered with the adjuvant composition. The antigen may be co-administered with the adjuvant composition comprising linear or branched unimolecular polymers linked to PRRa, wherein the polymers may be thermo-responsive. In one embodiment, the antigen may be linked to the polymer carrying PRRa. The antigen may be linked to the polymer carrying PRRa, wherein the polymers may be thermo-responsive.

The antigen may comprise a pathogen-derived antigen. The antigen may comprise a microbial antigen, such as a viral, parasitic, fungal, or bacterial antigen. The antigen may comprise a disease-associated antigen, such as a cancer/tumour-associated antigen. The tumour-associated antigen may comprise a self-antigen, such as gp120 or Na17 (melanoma). The tumour-associated antigen may comprise NY-ESO from testicular cancer. The tumour-associated antigen may be a mutated self-protein that is unique to the individual patient and contain neo-epitopes that are referred to as neoantigens that are patient-specific. The parasitic antigen may comprise a malarial antigen. The bacterial antigen may comprise a TB antigen. The antigen may comprise a Leishmania parasite antigen. In one embodiment, combinations of two or more antigens may be provided. The antigen may comprise HIV Envelope protein. The antigen may comprise a glycoprotein from Respiratory Syncytial Virus (RSV).

The PRR agonist molecules and/or antigens may be linked to the monomer units (such as co-monomer units) distributed along the polymer backbone at a density of between about 1 mol % and about 20 mol %. The PRR agonist molecules and/or antigens may also be linked to the end of the main polymer chain. The PRR agonist molecules and/or antigens may be linked to the polymer at a density of between about 5 mol % and about 20 mol %. The PRR agonist molecules and/or antigens may be linked to the polymer at a density of between about 5 mol % and about 100 mol %. The PRR agonist molecules and/or antigens may be linked to the polymer at a density of between about 5 mol % and about 80 mol %. The PRR agonist molecules and/or antigens may be linked to the polymer at a density of between about 5 mol % and about 50 mol %. The PRR agonist molecules and/or antigens may be linked to the polymer at a density of between about 8 mol % and about 20 mol %. The PRR agonist molecules and/or antigens may be linked to the polymer at a density of between about 8 mol % and about 15 mol %. The PRR agonist molecules and/or antigens may be linked to the polymer at a density of between about 8 mol % and about 10 mol %. The mol % of agonist and/or antigen is defined as the molar percentage of monomer units bearing the agonist and/or antigen incorporated to the main polymer chain. For example, 10 mol % agonist is equal to 10 monomer units linked to the agonist molecules from a total 100 monomer units. The remaining 90 may be macromolecule-forming monomeric units.

In one embodiment, the PRR agonist molecules and/or antigens may be linked to the monomer units at or substantially near one end of the polymer (i.e. semi-telechelic). In another embodiment, they may be linked to the monomer units at or substantially near both ends of the polymer (i.e. telechelic).

The PRR agonist and/or antigen may be covalently linked to the polymer. The PRR agonist and/or antigen may be linked to the polymer before particle formation. Additionally or alternatively, the link may be electrostatic (ion-ion), protein-protein interaction (e.g., coil-coil) and/or high affinity interaction between small molecules and proteins (e.g., biotin and avidin, as well as haptens and antibodies). The PRR agonist and/or antigen may be linked to the polymer by a linker molecule. The linker molecule may comprise an organic molecule. The organic linker molecule may comprise an aliphatic straight chain, branched or cyclic moiety. The organic linker molecule may comprise a C1-C18 alkane linker. The linker molecule may comprise a hydrophilic or hydrophobic linker. In one embodiment the linker is hydrophilic.

The linker may comprise PEG. The linker, such as PEG, may be at least 4 monomers in length. The linker, such as PEG, may be between about 4 and about 24 monomers in length, or more. Where the linker comprises a carbon chain, the linker may comprise a chain of between about 1 or 2 and about 18 carbons. Where the linker comprises a carbon chain, the linker may comprise a chain of between about 12 and about 18 carbons. Where the linker comprises a carbon chain, the linker may comprise a chain of between no more than 18 carbons.

The linker may be linked to the polymer backbone of the polymer by any suitable chemical moiety, for example any moiety resulting from a ‘click chemistry’ reaction, or thiol exchange chemistry. For example, a triazole group may attach the linker to the polymer. An alkyne group and an azide group may be provided on respective molecules to be linked by “click chemistry”. For example the antigen may comprise, or be modified with, an N-terminal azide that allows for coupling to a polymer having an appropriate reactive group such as an alkyne group. The skilled person will understand that there are a number of suitable reactions available to link the linking group to the polymer background. In one embodiment, the linker may be linked to the polymer backbone of the polymer by an amine. The link with an amine may be provided by reacting any suitable electrophilic group such as alkenes (via Michael addition), activated esters (for example, NHS ester), aldehydes, and ketones (via Schiff base).

The agonist and/or antigen may be linked to the polymer by a coil domain, split intein or tag, such as a SpyTag (for example taking advantage of a fibronectin-binding protein FbaB, which contains a domain with a spontaneous isopeptide bond between Lys and Asp).

Protein antigens are typically larger than 100 amino acids and typically require post-translational modification steps that require their production using in vitro expression systems. As such, in some circumstances it may not be easy to chemically incorporate “clickable”/bio-orthogonal groups, which allow for site-specific attachment into proteins. Instead, recombinant technologies can be used express antigens as fusion proteins with coil domains, split inteins and Spy tags that permit site-selective docking to polymeric platforms.

According to another aspect of the present invention, there is provided an immunogenic composition comprising an adjuvant and an antigen according to the invention herein. The immunogenic composition may be a vaccine.

The antigen may be separate, or linked to the polymer. In embodiments where the antigen is linked to the polymer, it may be releasable from the polymer by degradation, such as chemical or enzyme mediated degradation.

According to another aspect of the present invention, there is provided a method of treatment or prevention of a disease comprising the administration of an adjuvant or immunogenic composition according to the invention herein to a subject in need thereof.

According to another aspect of the present invention, there is provided a method of eliciting an immune response for a disease comprising the administration of an adjuvant or immunogenic composition according to the invention herein to a subject in need thereof.

The method may comprise the step of forming particles of the adjuvant in the subject by the action of a temperature shift from the administered adjuvant or immunogenic composition moving from outside the body to inside the body of the subject.

The adjuvant technology described herein may be used as direct immunotherapeutic agents by activating immune cells and reversing regulatory T cell tolerance. Alternatively, the invention may be used to improve host immune responses against cancer through vaccination.

The disease may be any disease suitable for treatment or prevention by vaccination. The disease may be an infectious disease. The disease may be cancer. The infectious disease may comprise any of a bacterial infection, viral infection, fungal infection, or parasite infection. The disease may comprise any of the group selected from malaria, cancer, tuberculosis, or parasitic disease; or combinations thereof. The parasite may comprise Leishmania parasite.

The disease may comprise any disease selected from the group comprising localized and metastatic cancers of the breast, such as infiltrating ductal, invasive lobular or ductal/lobular pathologies; localized and metastatic cancers of the prostate; localized and metastatic cancers of the skin, such as basal cell carcinoma, squamous cell carcinoma, Kaposi's sarcoma or melanoma; localized and metastatic cancers of the lung, such as adenocarcinoma and bronchiolaveolar carcinoma, large cell carcinoma, small cell carcinoma or non-small cell lung cancer; localized and metastatic cancers of the brain, such as glioblastoma or meningioma; localized and metastatic cancers of the colon; localized and metastatic cancers of the liver, such as hepatocellular carcinoma; localized and metastatic cancers of the pancreas; localized and metastatic cancers of kidney, such as renal cell carcinoma; and localized and metastatic cancers of the testes.

The disease may comprise any viral disease caused by a viral agent selected from the group comprising influenza, human immunodeficiency virus (HIV), Ebola, coronaviruses (such as MERS, SARs), cytomegalovirus, mumps, measles, rubella, polio, enterovirus, parvovirus, Herpes Simplex Virus (HSV), Arboviruses (eastern equine, western equine, St. Louis, Venezuelan equine encephalitis, and West Nile viruses), varicella-zoster virus, Epstein-Barr virus, and Human Papilloma Virus (HPV).

The disease may comprise any bacterial disease caused by a bacterial agent selected from the group comprising Mycobacterium tuberculosis, Staphylococcus aureus, Streptococcus pneumoniae, Enterococci, Pseudomonas aeruginosa, Clositridium difficilie, Treponema Pallidum (Syphilis), and Chlamidia Trachomatis.

The disease may comprise any protozoan disease caused by a protozoan agent selected from the group comprising Plasmodia parasites that cause Malaria (e.g. Plasmodium falciparum and Plasmodium vivax), parasites that cause Leishmaniasis (e.g., Leishmania major), the parasite that causes Chagas disease (Trypanosoma cruzi), and parasites that cause Giardiasis (Giardia lablia).

The invention herein may be used to treat or prevent conditions associated with a toxin. The toxin may be a protein-based toxin produced by bacteria, such as Anthrax or Tetanus toxins. The toxin may be a “Manmade”/artificial toxin, for example related to drug abuse. The toxin may comprise a protein toxin (such as ricin), a small molecule toxin (e.g., Sarin), or a small molecule drug of abuse (e.g., di-acetylated morphine/heroine).

The subject may be a mammal. The mammal may be a human.

The method may further comprise the administration in combination with another active agent, such as a therapeutic molecule, biologic or different antigen. In an embodiment where a PRR agonist is linked to a polymer, the adjuvant may be administered concurrently with an antigen. The antigen may be mixed with the adjuvant prior to administration. In an embodiment where an antigen is linked to the polymer, the adjuvant may be administered concurrently with the PRR agonist. The PRR agonist may be mixed with the adjuvant prior to administration.

According to another aspect of the present invention, there is provided an adjuvant or immunogenic composition according to the invention herein, for use in the prevention or treatment of a disease.

The adjuvant or immunogenic composition may be used in combination with at least one other therapeutic or preventative active agent.

The use may be for use as a vaccine. The immune response may be a protective immune response, for example it may completely prevent or cure the disease, or may at least alleviate symptoms of the disease.

The administration may be into a specific tissue site in a subject. The administration may be intramuscular. The administration may be any of intramuscular, subcutaneous, transcutaneous, or oral. Alternatively, the administration may be systemic, for example, when tumours are treated with the polymer arranged to form particles at the site of the tumour. A dose of about 0.1-10 mg of the adjuvant may be administered.

According to another aspect of the present invention, there is provided a method of preparing an adjuvant comprising polymer particles, the method comprising the steps of:
providing an adjuvant composition according to the invention;
filter sterilising the adjuvant composition; and
forming adjuvant particles by providing a temperature shift from below the lower critical solution temperature of the polymer to above the lower critical solution temperature of the polymer.

The particles may be formed in situ (e.g., after administration to a subject). The temperature shift may be provided by administration of the adjuvant into a subject (e.g., an increase in temperature to body temperature, or temperature of tissue inflammation at a specific site in the body of the subject). The temperature shift may be provided post-administration of the adjuvant, by radiation directed into a specific tissue of the subject, such as a tumour tissue. Alternatively, the particles may be formed prior to administration, where the adjuvant is heated, for example in a water bath.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

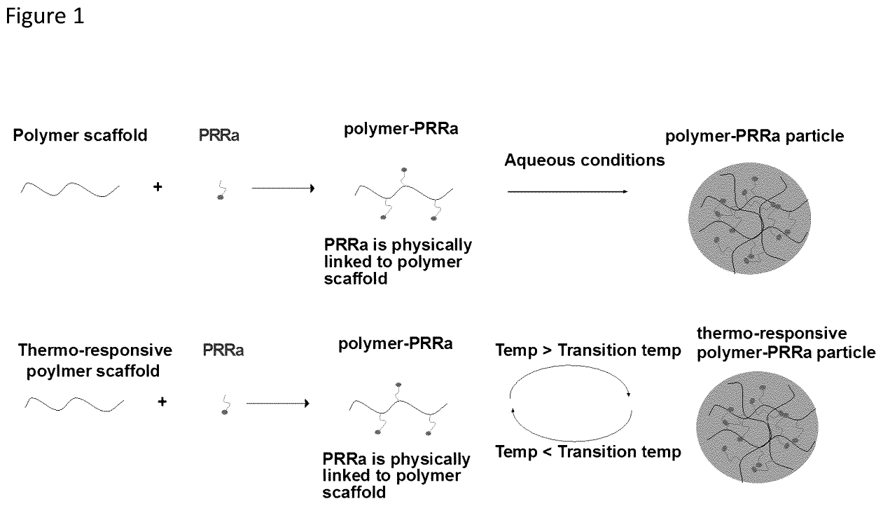

FIG. 1: Summary of present invention. The novelty of the present invention is conveyed in this diagram. The invention relates to an adjuvant composition comprised of PRRa linked to linear or branched polymer chains that exist as unimolecular polymers that undergo particle formation in aqueous media due to their hydrophobic properties or undergo particle formation in response to an external stimuli, such as temperature, wherein the polymer is a thermo-responsive polymer. Note that adjuvant composition can be stored as unimolecular polymer chains linked to PRRa but provide the advantage that the polymer chains can assemble into particles in physiologic conditions in room temperature in aqueous buffers or at body temperature in vivo.

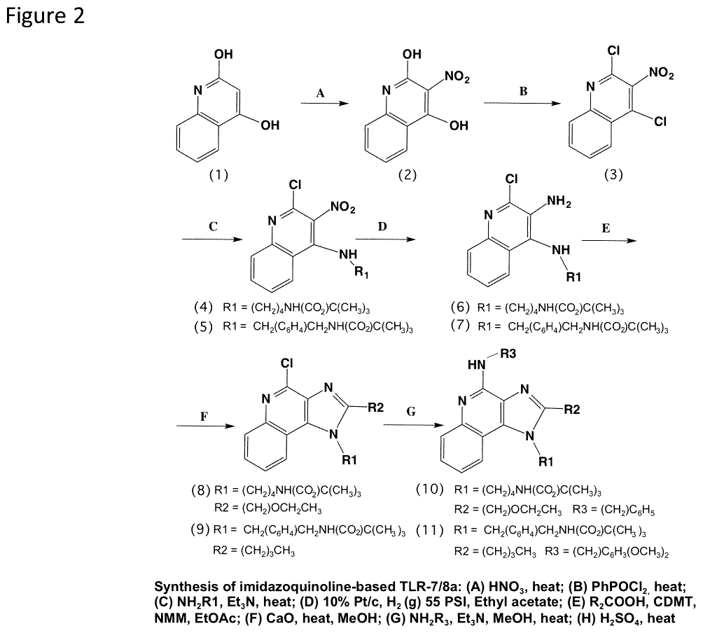

FIG. 2: Synthesis of imidazoquinoline-based polymer-reactive TLR-7/8a used as model PRRa in these studies.

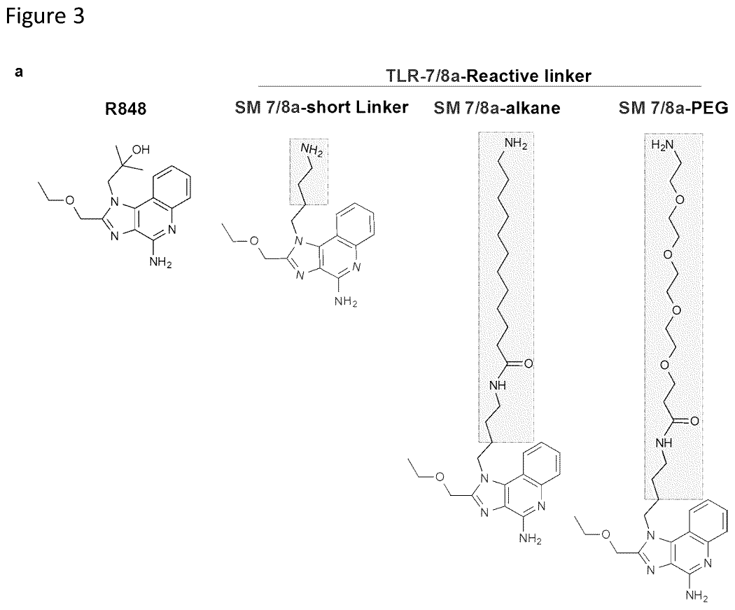

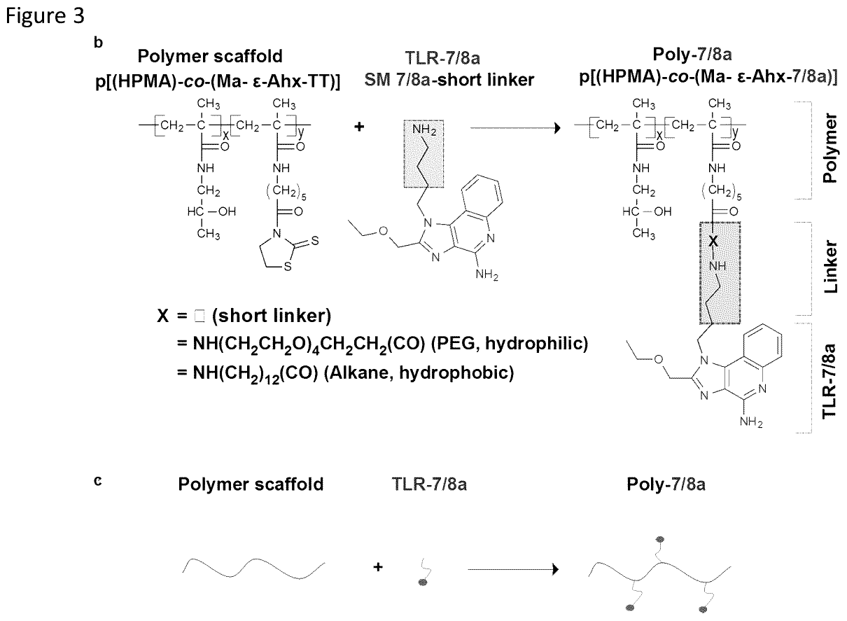

FIG. 3: Synthesis of Poly-7/8a. (a) Chemical structures of TLR-7/8a: Nucleophilic analogs of the commercially available TLR-7/8a, R848, were produced by replacing the isopropanol group with reactive linkers that are indicated by the shaded boxes overlaying the structures of SM 7/8a, SM 7/8a-alkane and SM 7/8a-PEG. Note that the alkane and PEG linkers are of comparable length but different composition (hydrophobic vs. hydrophilic). The terminal amine on each of the linkers permitted facile attachment to amine reactive polymer precursors. (b) Poly-7/8a were generated by reacting nucleophilic TLR-7/8a (SM 7/8a) with HPMA-based copolymers in a facile one step reaction, resulting in a stabile amide bond between the TLR-7/8a and the polymer backbone. Note that the brackets represent repeating units of each monomer, with the subscripts, x and y, representing the percentage composition (mol %) of each monomer. (c) Schematic of the reaction used to generate Poly-7/8a. Poly=polymer; SM=small molecule; HPMA=N-(2-hydroxypropyl) methacrylamide; MA=methacrylamide; Ahx=aminohexanoic acid; PEG=Polyethylene glycol; TT=2-Thiazolidine-2-thione

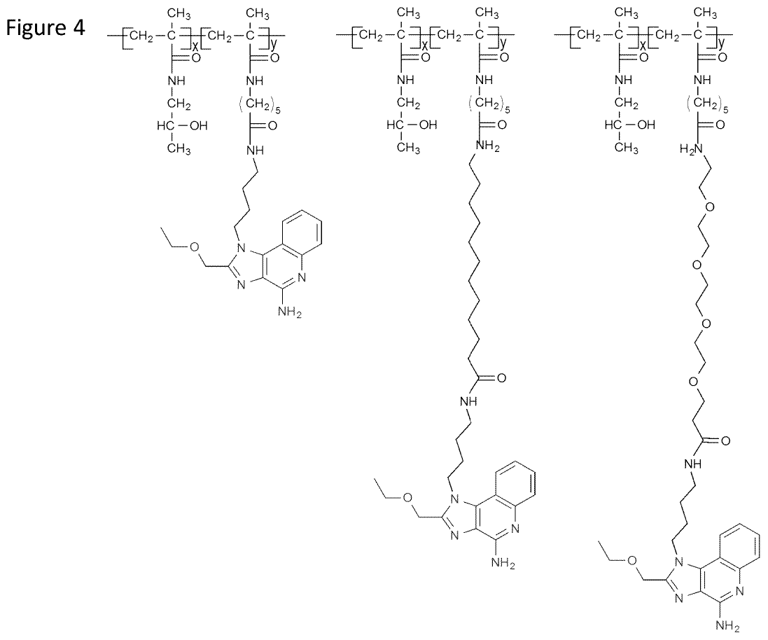

FIG. 4: HPMA-based carriers of TLR-7/8a.

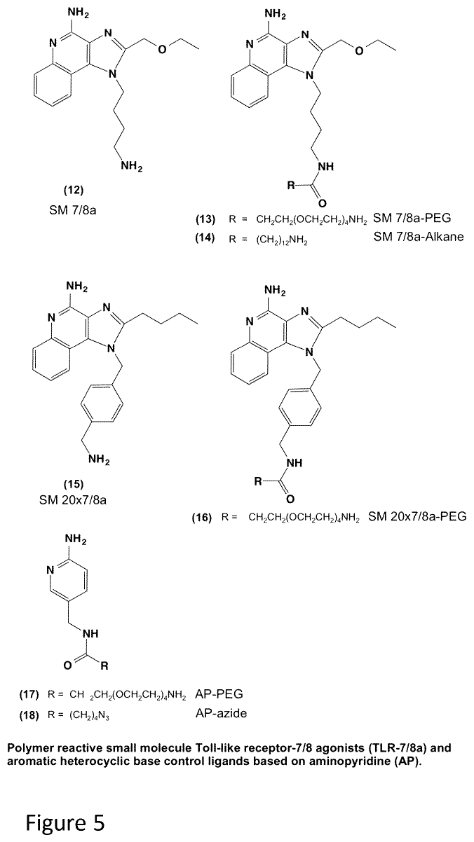

FIG. 5: Additional conjugatable TLR-7/8a and control ligands.

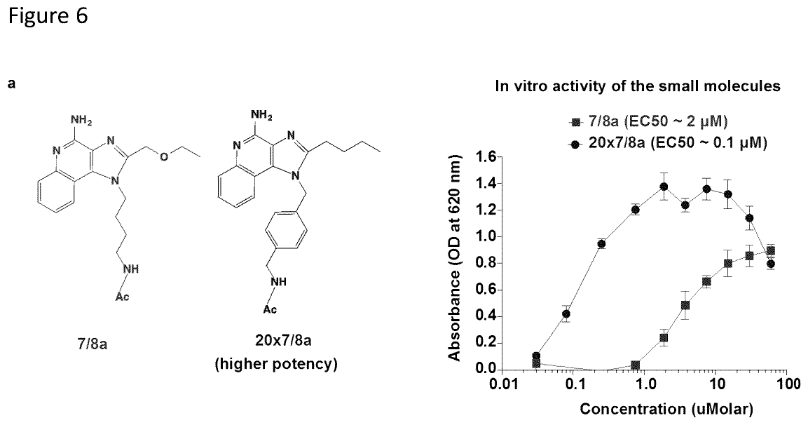

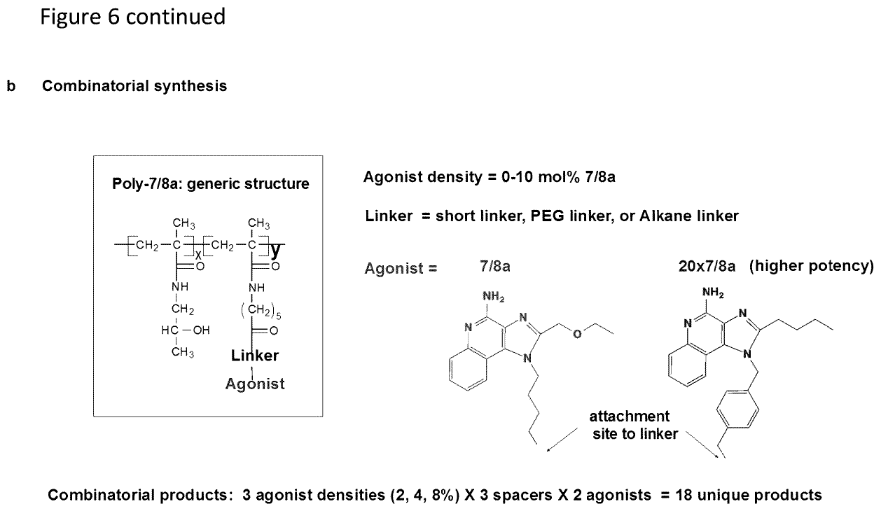

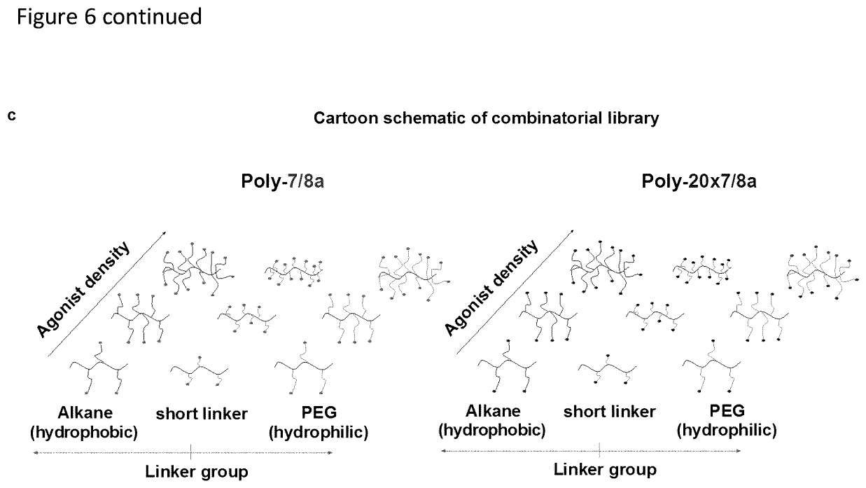

FIG. 6: Combinatorial synthesis was used to generate diverse arrays of Poly-7/8a. (a) In addition to SM 7/8a described previously, a ˜20-fold more potent TLR-7/8, referred to as SM 20×7/8a, was attached to Poly-7/8a. The potency of these two TLR-7/8a was evaluated in vitro using HEK293 hTLR7 reporter cells; absorbance at 405 nm is proportional to TLR7 binding. (b) A combinatorial library of Poly-7/8a was generated by attaching 2 unique TLR-7/8a (SM 7/8a or SM 20×7/8a) to reactive HPMA-based copolymers at different densities (˜2,4, 8 mol %) using either a short, alkane or PEG linker. By reacting 2 unique TLR-7/8a, at 3 different densities, using 3 different linkers, there are 18 unique products that can be generated, as illustrated (c). Note that this cartoon representation is for illustrative purposes; not all Poly-7/8a represented in this schematic were evaluated in this study, nor does this schematic represent all the materials described herein.

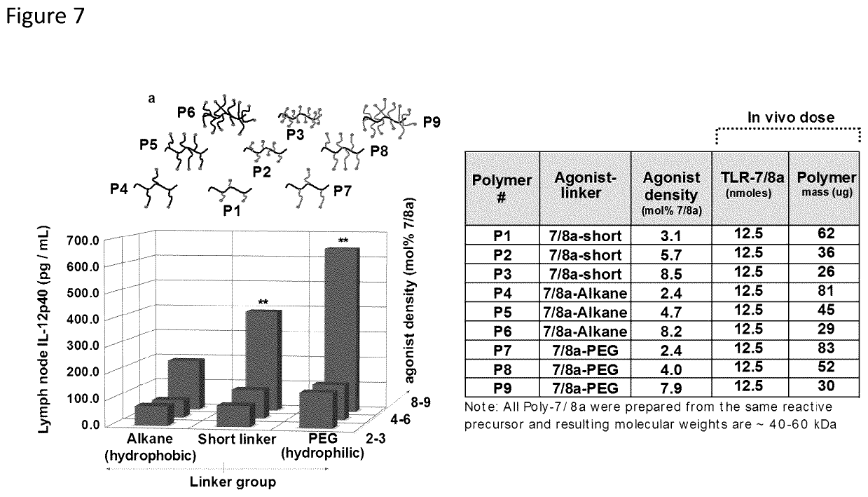

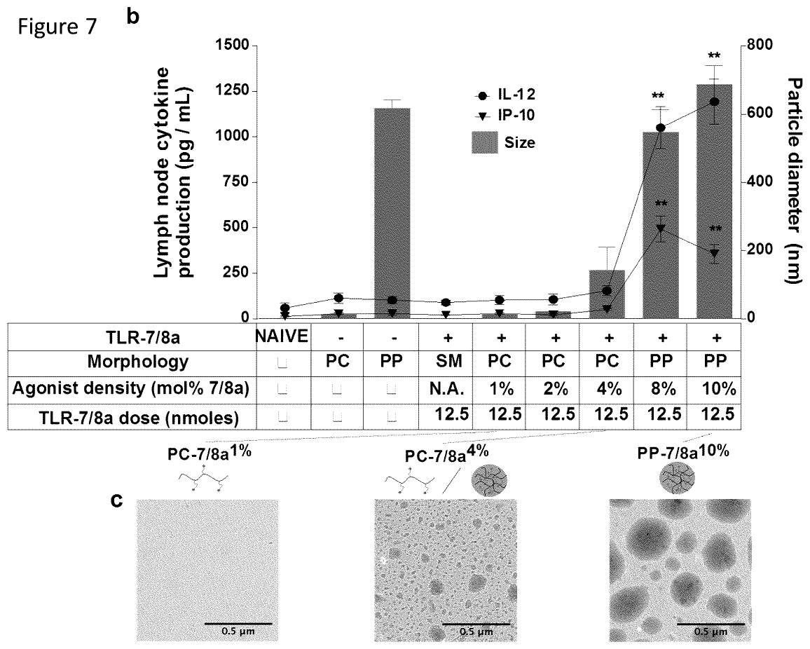

FIG. 7: In vivo screening of a combinatorial library of Poly-7/8a yields structure-activity insights. (a) Lymph node (n=4) IL-12p40 induced by a combinatorial library of Poly-7/8a. (b) Influence of TLR-7/8a density on lymph node (n=6) IL-12p40 and IP-10 levels (left y-axis, line graphs) and particle size assessed by dynamic light scattering (right y-axis, bar graph). (c) Cryo-electron microscopy of selected samples is shown for illustrative purposes. All data are represented as mean±SEM; statistical significance is relative to SM 7/8a and polymer controls (ANOVA with Bonferroni correction); ns, not significant (P>0.05); *, P<0.05; **, P<0.01. SM=small molecule; PC=polymer coil; PP=polymer particle.

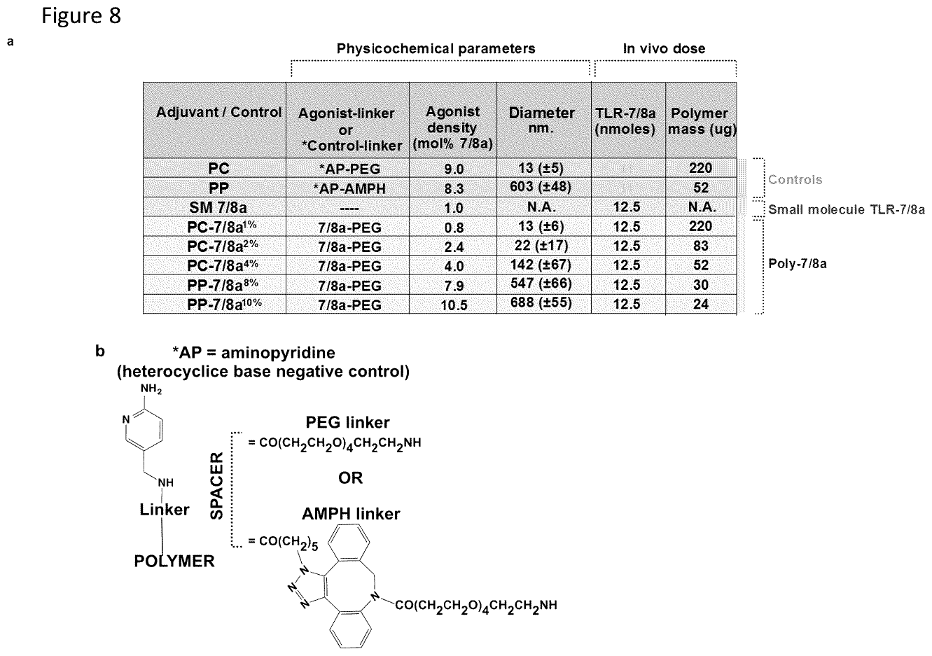

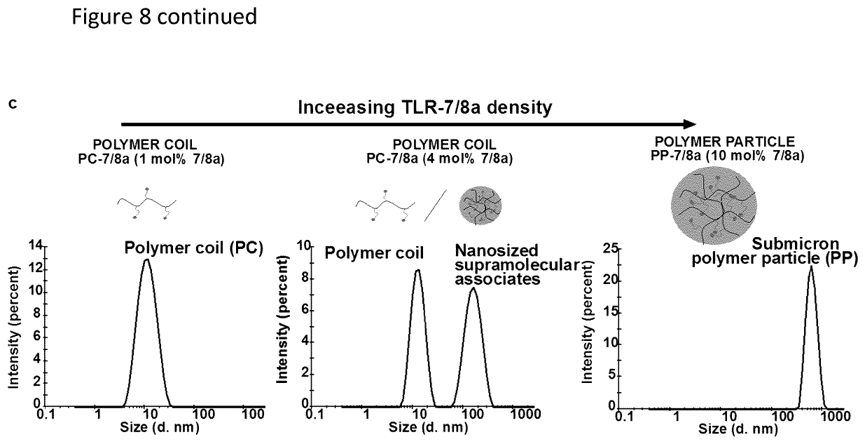

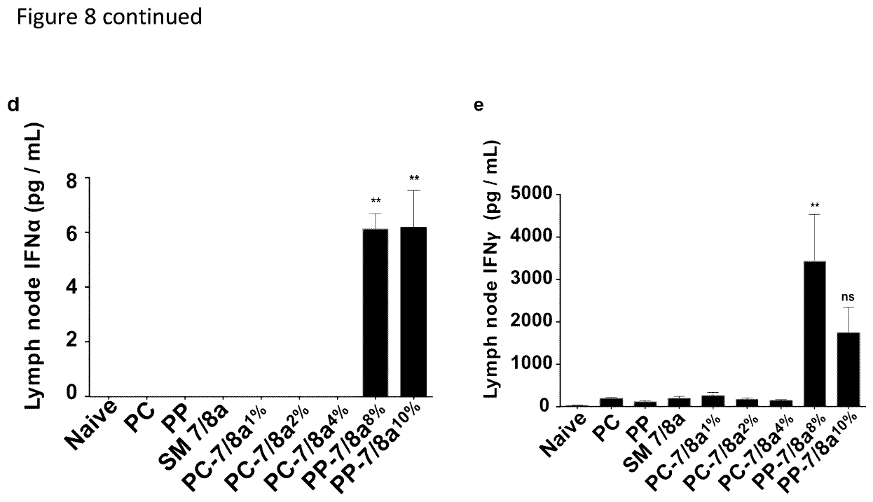

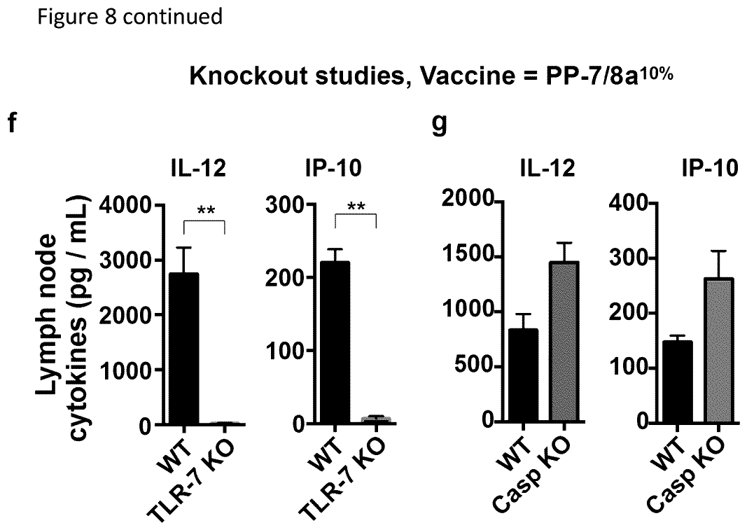

FIG. 8: Increasing density of agonists on polymers results in an increased tendency of Poly-7/8a to form particles in aqueous solutions and is associated with enhanced local innate immune activity. (a) Properties of Poly-7/8a, SM 7/8a and control polymers are summarized in the table. (b) Negative control polymers were generated using aminopyridine (AP) to account for the contribution of the aromatic amine present on the imidazoquinoline based TLR-7/8a used in this study. AP was attached to polymers either through a PEG or amphiphilic (AMPH) spacer. (c) The relationship between TLR-7/8a density and particle formation was characterized by light scattering: Polymers with 1 mol % TLR-7/8a attached (Poly-7/8^{1%}) exist as polymers coils, referred to as PC; Poly-7/8a^{4%} exist in equilibrium between polymer coils and nano-sized supramolecular associates; and, Poly-7/8a^{10%} assemble into submicron polymer particles, referred to as PP. (d, e) Poly-7/8a, SM 7/8a or polymer controls were subcutaneously administered into both hind footpads of C57/BL6 mice. 24 h after administration, supernatant of overnight lymph node cell suspensions were assessed for IFNα (d) or IFNγ (e) by ELISA. (f-g) Lymph nodes were isolated from wild type (WT), TLR-7 knockout or Caspase 1/11 (inflammasome) knockout mice 24 h after subcutaneously administering Poly-7/8a^{10%}; culture supernatants were evaluated for IP-10 and IL-12 by ELISA. All data are represented as mean of replicates from individual experiments±SEM; statistical significance is relative to all other groups, unless specified otherwise within the figure (ANOVA with Bonferroni correction, n=4); ns, not significant (P>0.05); *, P<0.05; **, P<0.01.

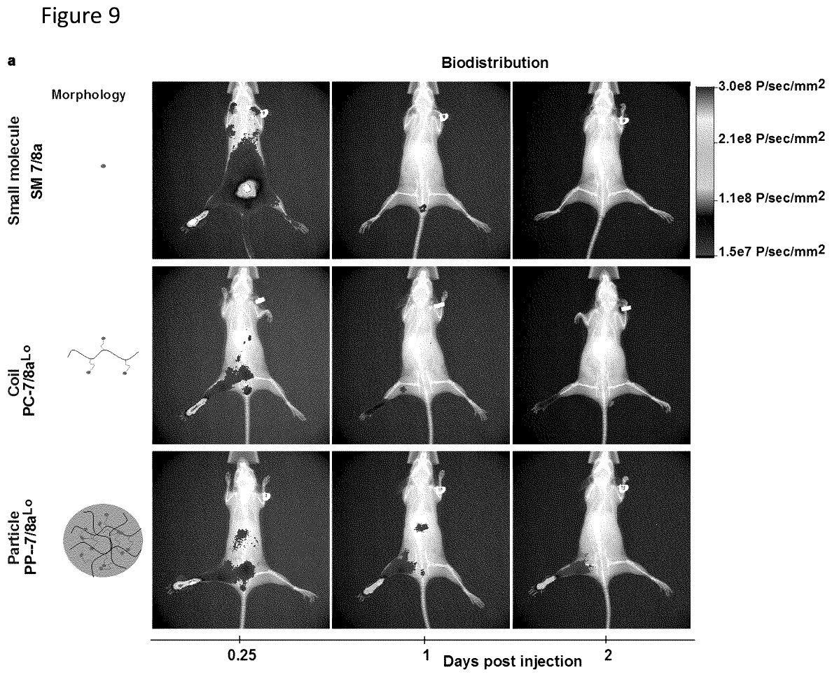

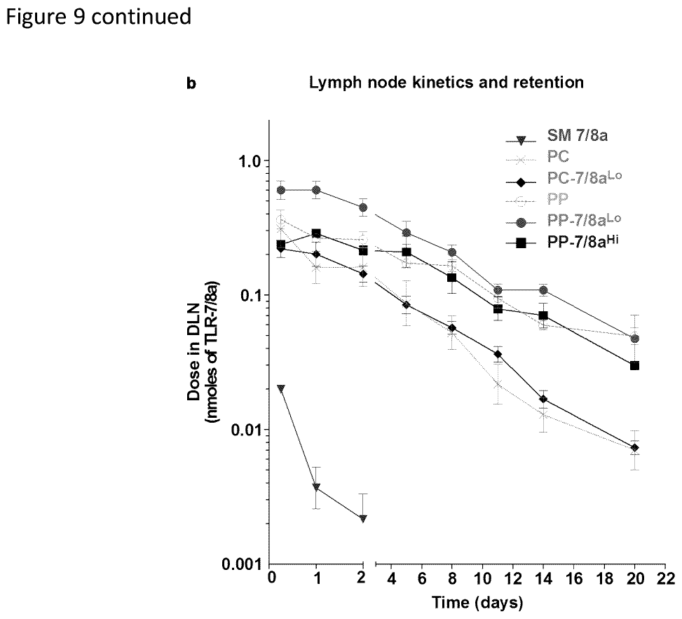

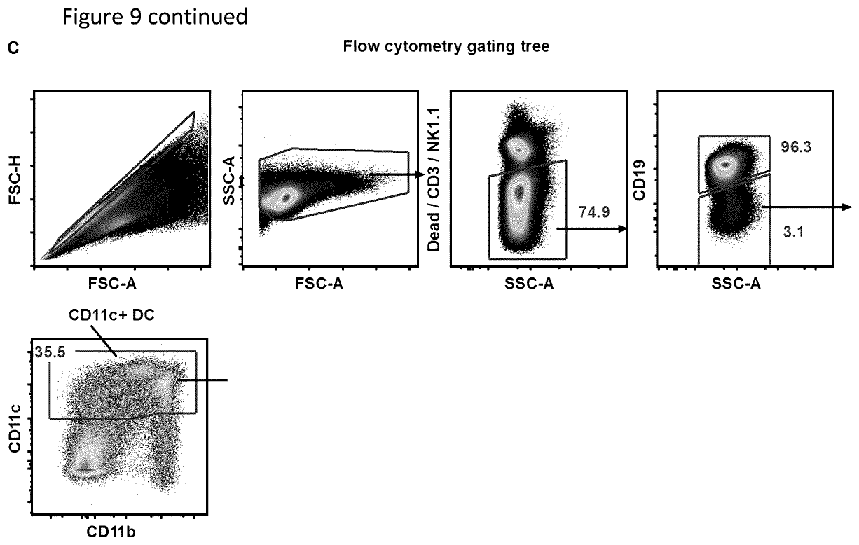

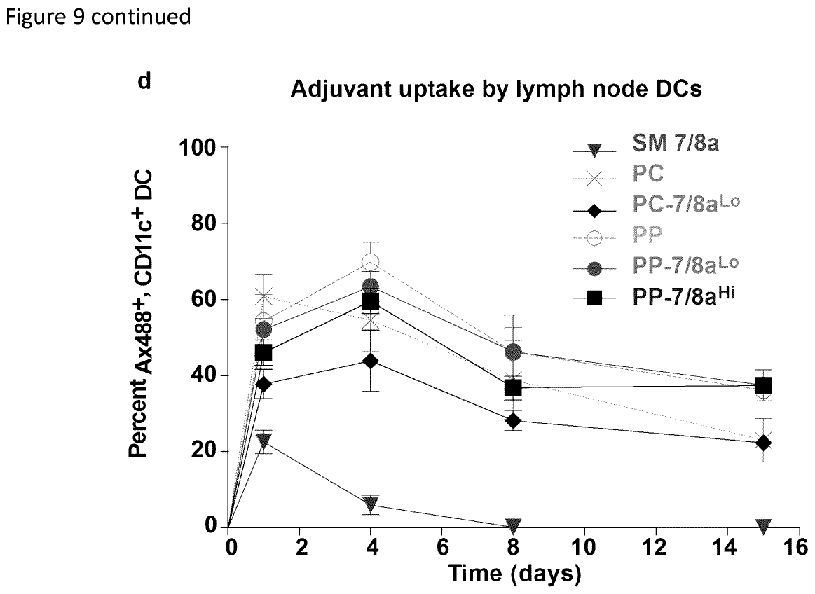

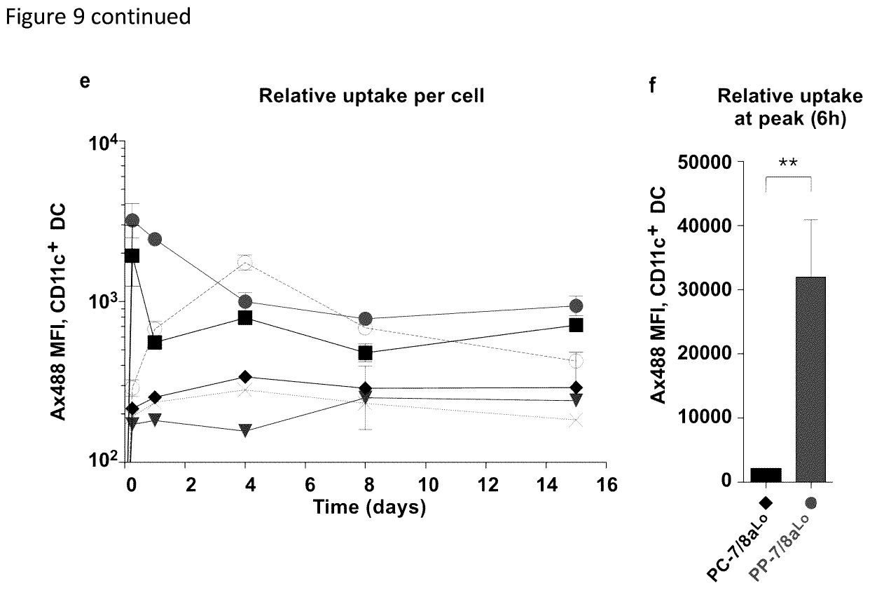

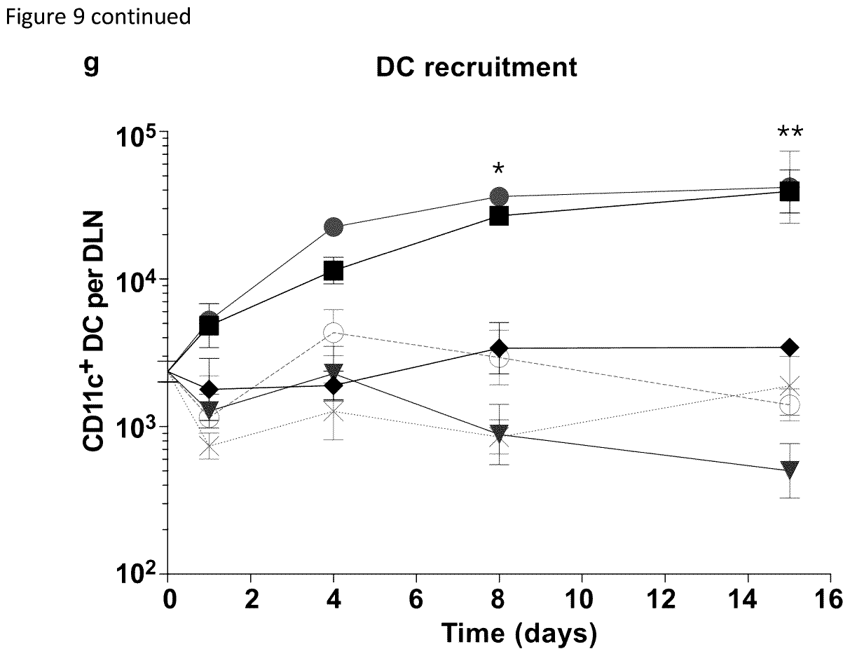

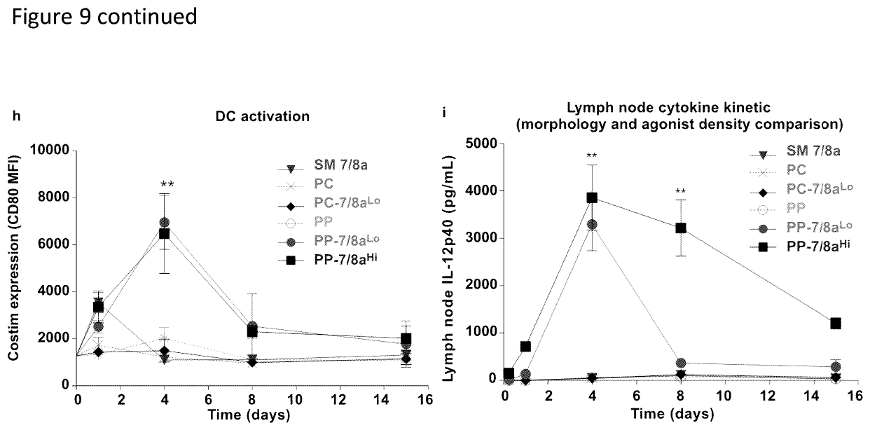

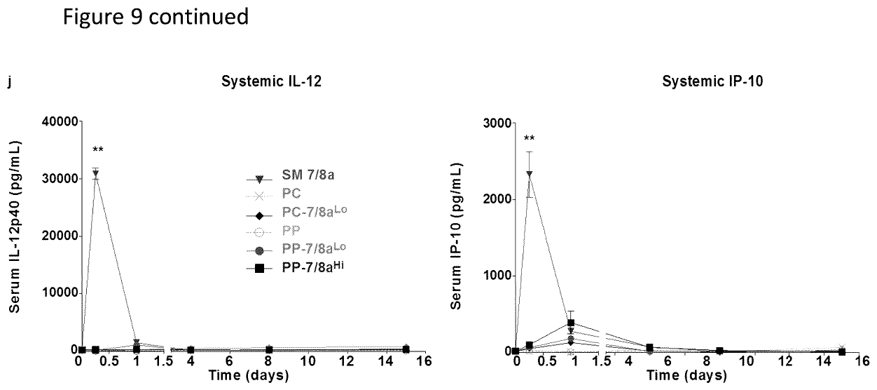

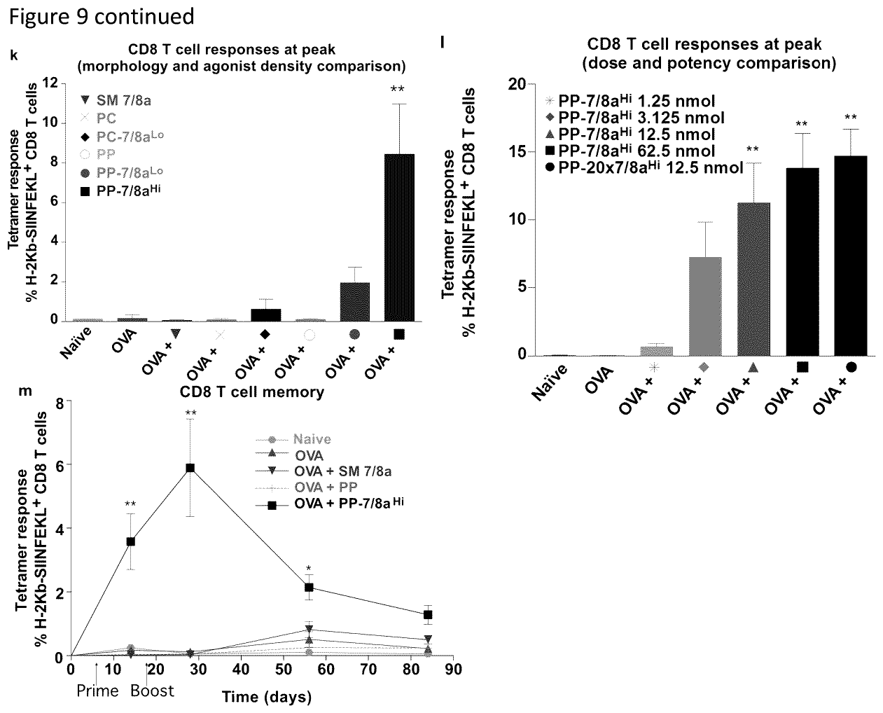

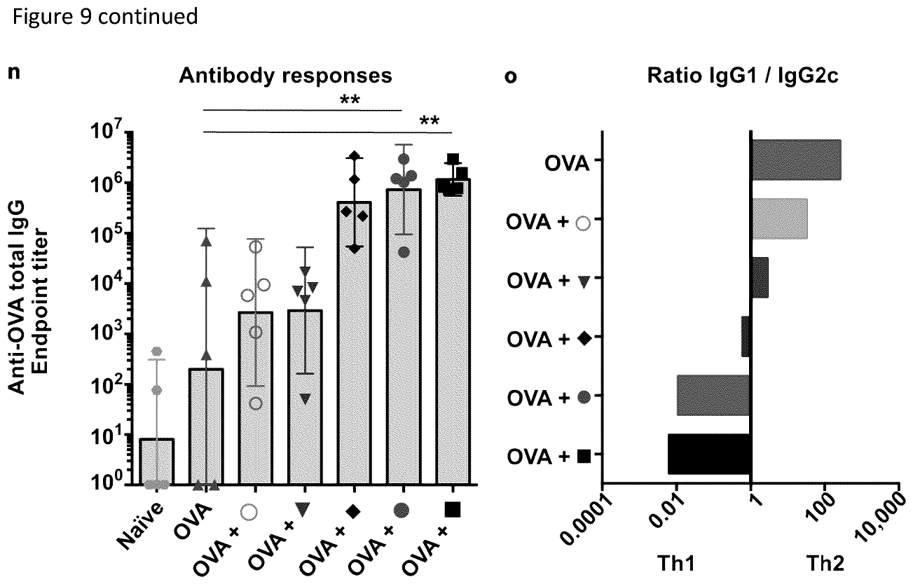

FIG. 9: Morphology of the carrier and TLR-7/8a agonist density, dose and potency independently influence the magnitude and spatiotemporal characteristics of innate immune activation. (a-g) To facilitate in vivo tracking, dye-labeled Poly-7/8a (PP-7/8a^{Hi}, PP-7/8a^{Lo}, PC-7/8a^{Lo}) (Note PP-7/8a^{Hi}=10 mol % and PP-7/8a^{Lo}=3 mol %) and small molecule TLR-7/8a normalized for TLR-7/8a dose (12.5 nmol), and controls, were delivered subcutaneously to the left footpad of mice. (a, b) mice (n=3) that received IR-dye labeled materials were imaged by dual modality X-ray and epifluorescence spectroscopy; (a) Representative images illustrating biodistribution over the first 2 days; (b) Kinetics of the different constructs in the popliteal lymph node. (c-e) Draining lymph nodes (n=3) were harvested at serial timepoints and evaluated by flow cytometry. (c) example gating tree. Total CD11c⁺ DC were evaluated for (d) adjuvant uptake (% Ax488⁺), (e,f) relative adjuvant uptake per cell (Ax488 MFI; note that PP-7/8a^{Lo} and PC-7/8a^{Lo} are matched for TLR-7/8a density and dose), (g) magnitude and (h) activation (CD80 co-stimulatory molecule expression). (i) IL-12p40 was measured from the supernatant of ex vivo lymph node cultures. (j) IL-12p40 and IP-10 was measured in the serum; note that the polymers do not induce systemic cytokine production following local (subcutaneous) adjuvant administration. (k-o) Poly-7/8a, SM 7/8a or a control were formulated with 50 μg of OVA in PBS and given subcutaneously to C57/BL6 mice (n=5) at days 0 and 14. At day 28, tetramer+CD8 T cell responses were evaluated from whole blood. (m) Durability of tetramer+CD8 T cell responses was followed out to 12 weeks. (n) Total IgG antibody titers and the ratio of IgG1/IgG2c antibody endpoint titers were evaluated from sera on day 28. All data are represented as mean of replicates±SEM; statistical significance is relative to small molecule TLR-7/8a and polymer controls (ANOVA with Bonferroni correction); ns, not significant (P>0.05); *, P<0.05; **, P<0.01.

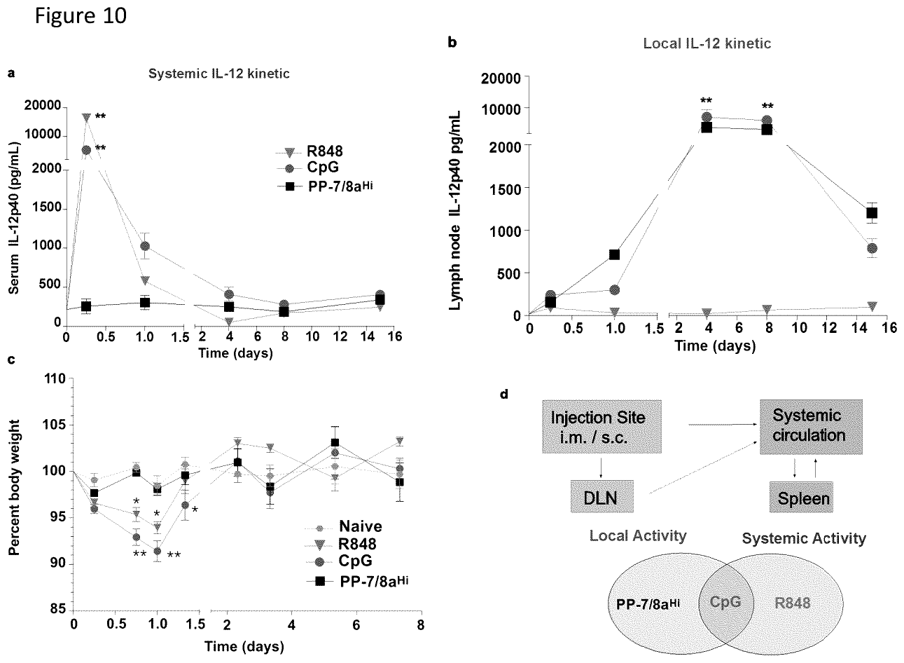

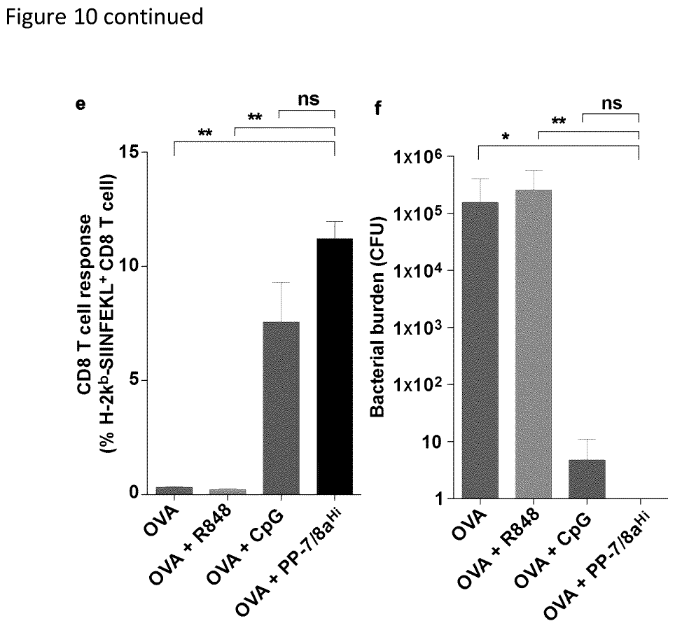

FIG. 10: Persistent local innate immune by particulate Poly-7/8a (PP-7/8a) activation is necessary and sufficient for inducing protective CD8 T cell responses. (a-c) CpG (20 μg), R848 (62.5 nmol) or PP-7/8a^{Hi} (62.5 nmol) were delivered subcutaneously into both hind footpads of C57/BL6 mice. (a) Supernatant of ex vivo cultured lymph node cell suspensions (n=4) were evaluated for IL-12p40 by ELISA at serial timepoints. (b) Serum (n=3-5) was assessed for IL-12p40 by ELISA at serial timepoints. (c) Percent body weight change (n=3) following subcutaneous administration of different vaccine adjuvants. (d) A model for understanding the relationship between biodistribution and local and systemic innate immune activation. (e, f) C57/BL6 mice (n=6) received a single subcutaneous footpad injection of 50 μg of OVA formulated with adjuvant at days 0 and 14. (e) At day 25, tetramer⁺ CD8 T cell responses from whole blood were evaluated by flow cytometry. (f) Mice (n=6) were challenged intravenously at day 28 with LM-OVA and bacterial burden in the spleens was evaluated on day 31. (g, h) Immunogenicity experiments were repeated using SIV Gag p41 in place of OVA. All data are represented as mean±SEM; statistical significance is relative to naïve, unless individual comparisons are indicated (ANOVA with Bonferroni correction); ns, not significant (P>0.05); *, P<0.05; **, P<0.01.

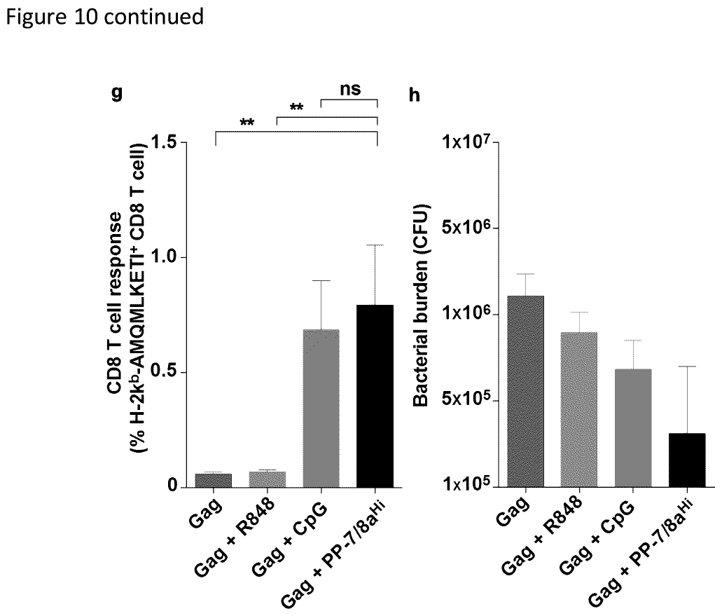

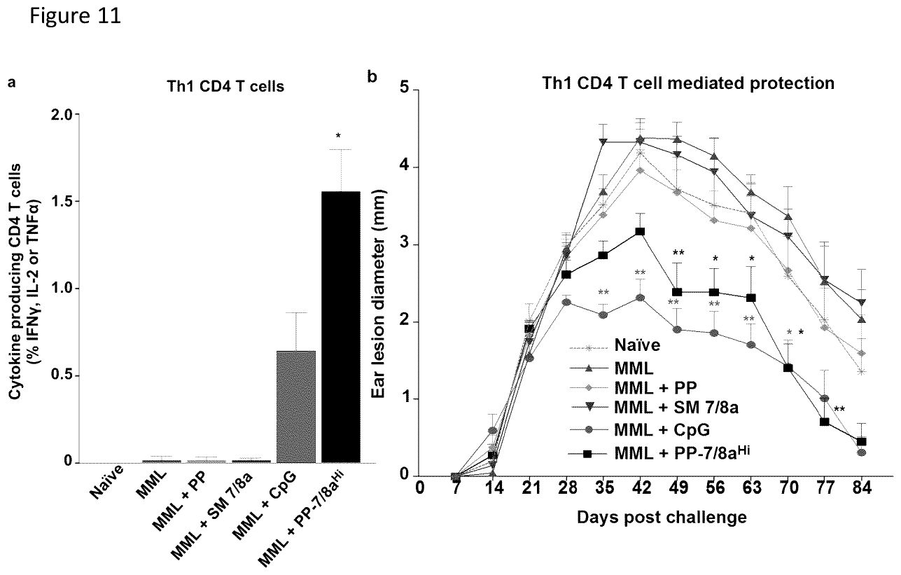

FIG. 11: Particulate Poly-7/8a induce Tₕ1 CD4 T cells that mediate protection against Leishmania major. C57/BL6 mice received subcutaneous immunizations of 20 μg of MML protein either alone, or formulated with an adjuvant, on days 0, 21 and 42. (a) Splenocytes were isolated on day 70 and stimulated in vitro with MML peptide pool. Antigen-specific cytokine producing CD4 T cells in the mixed splenocyte cultures were quantified by flow cytometry (n=5) for their capacity to produce Tₕ1 characteristic cytokines (IFNγ, IL-2 and TNFα). (b) Mice (n=6) were challenged intradermally in both ears with L major at day 70. Ear lesion diameters were measured for 12 weeks. All data are represented as mean of replicates±SEM; statistical significance is relative to naïve, MML alone and SM 7/8a (ANOVA with Bonferroni correction); ns, not significant (P>0.05); *, P<0.05; **, P<0.01.

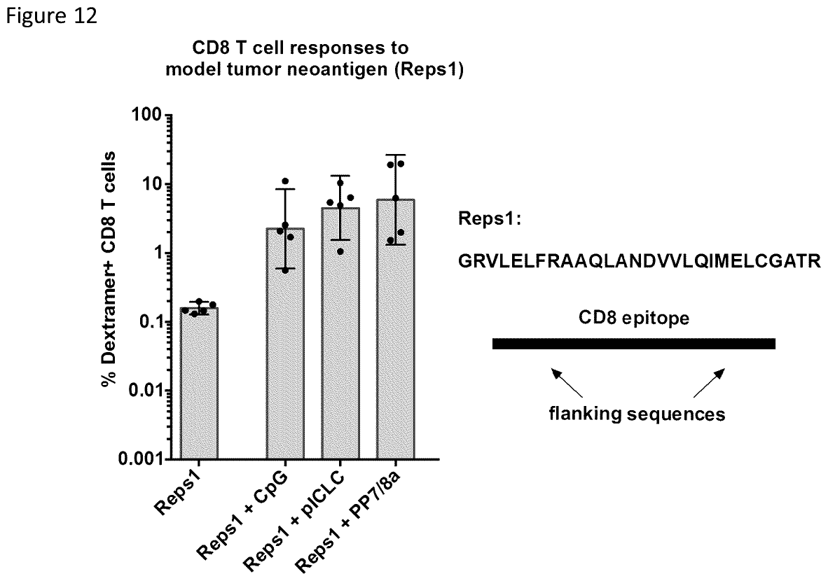

FIG. 12: Particulate Poly-7/8a (PP-7/8a) induces CD8 T cell responses against peptide-based tumor neoantigens. The activity of PP-7/8a for inducing CD8 T cell immunity against the model tumor neoantigen, Reps1, was benchmarked against CpG and pICLC. PP-7/8a (4 nmol), CpG (3.1 nmol) and pICLC (20 μg) were administered with 4 nmol of Reps 1 (peptide sequence shown) subcutaneously into the footpad of mice (n=5). Dextramer positive CD8 T cell responses were determined 2 weeks after two immunizations.

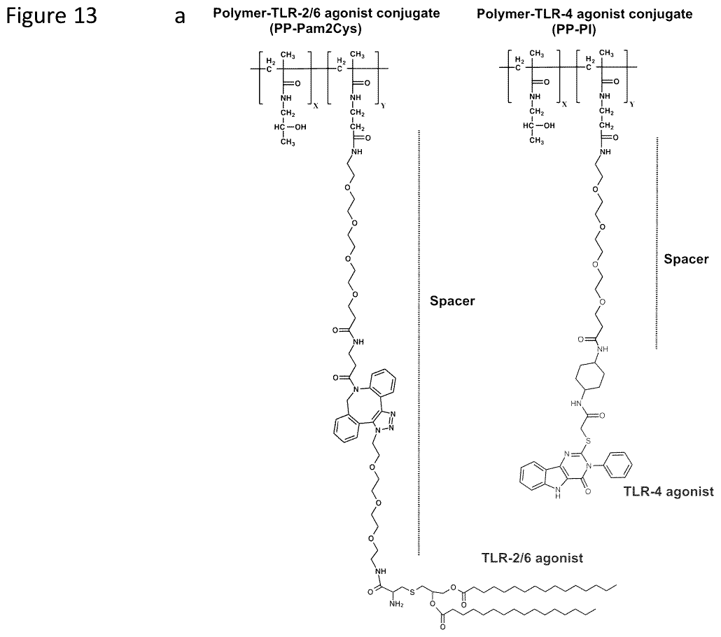

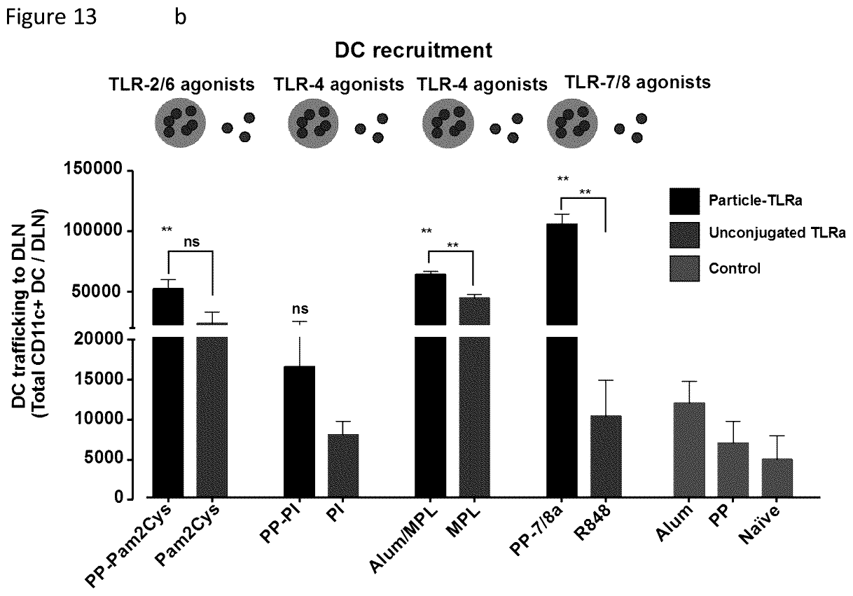

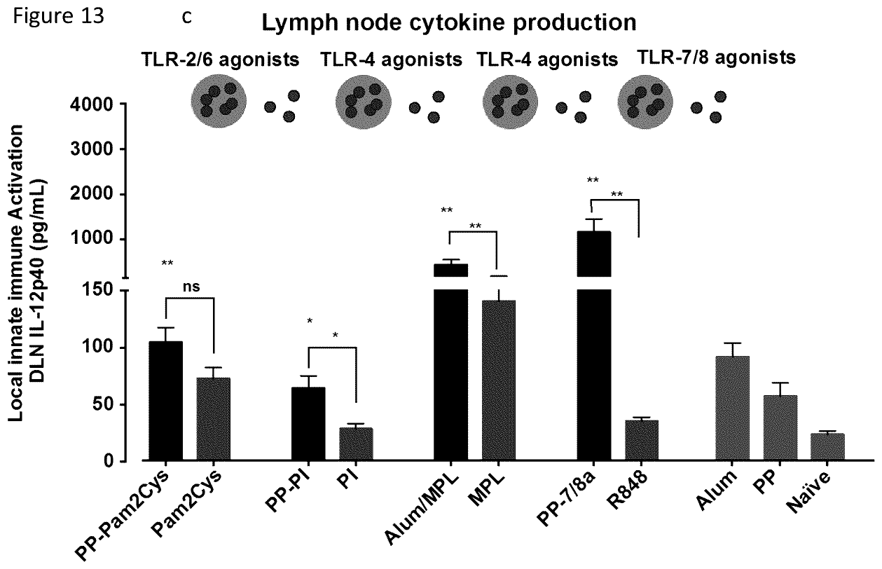

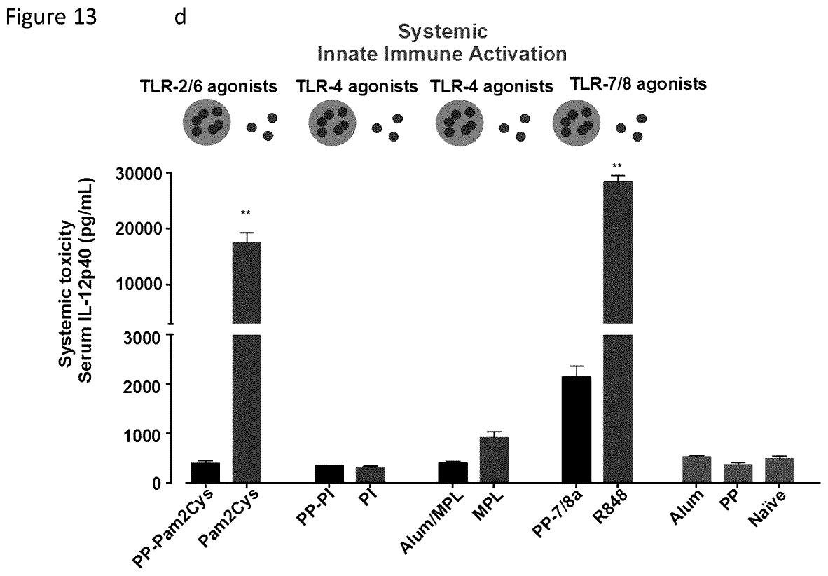

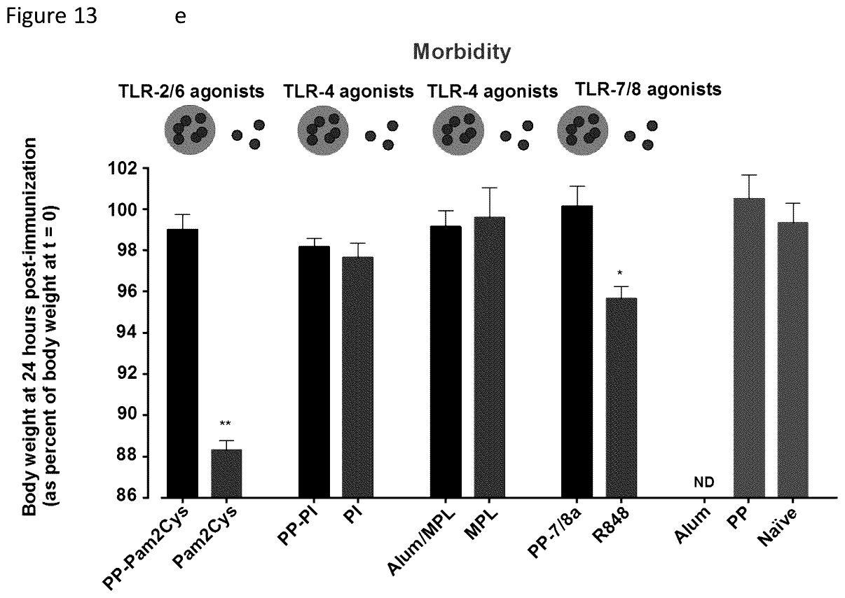

FIG. 13: Polymer particle (PP) carriers of other PRRa enhance lymph node innate immune activity and reduce systemic toxicity. (a) HPMA-based polymer carriers of the TLR-2/6a Pam2Cys (PP-P2Cys) and a pyrimidoindole-based TLR-4a (PP-PI). Both Pam2Cys and PP-PI were prepared with >5 mol % TLRa to promote particle formation in aqueous conditions. (b-e) The various different PP-TLRa conjugates or unconjugated TLRa were administered into the hind footpads of mice and evaluated for (b) DC recruitment to draining lymph nodes (n=3), (c) IL-12p40 production in lymph nodes (n=8), (d) serum IL-12p40 production (n=5) and (e) body weight reduction. All data are represented as mean±SEM; statistical significance is relative to naïve, unless individual comparisons are indicated (ANOVA with Bonferroni correction); ns, not significant (P>0.05); *, P<0.05; **, P<0.01.

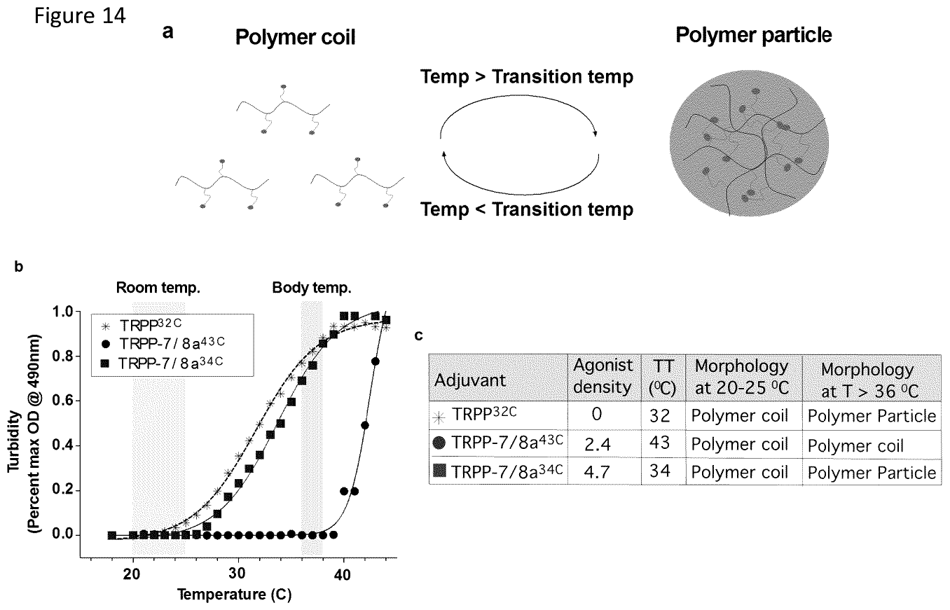

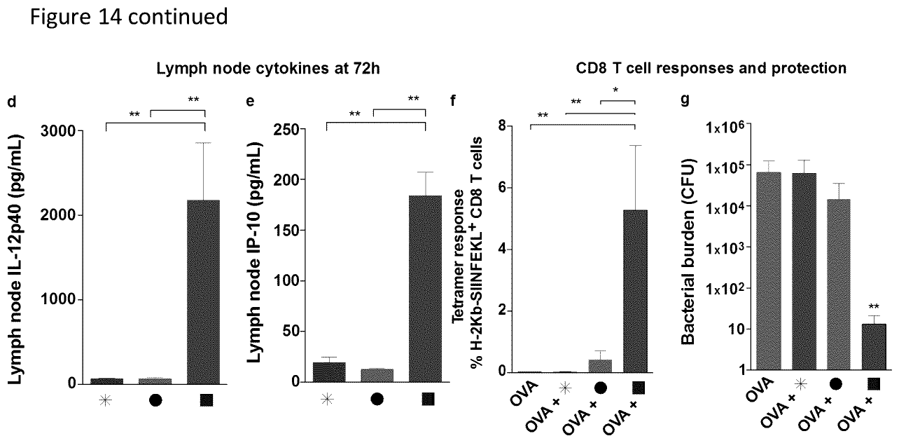

FIG. 14: Thermo-responsive polymer particles (TRPP) permit in vivo particle assembly that leads to persistent innate immune activation sufficient for eliciting protective CD8 T cell responses. (a) Schematic of TRPP shown reversibly assembling into particles. (b) Transition temperatures (TT) were empirically determined by measuring the turbidity (OD at 490 nm) of solutions of TRPP in PBS at different temperatures. (c) Table summarizing the thermo-responsive properties of select TRPP. (d and e) TRPP-7/8a and TRPP control were delivered subcutaneously into both hind footpads of C57/BL6 mice. Popliteal lymph nodes (n=4) were harvested at 72 h and cultured ex vivo overnight. Supernatants were evaluated for the presence of (d) IL-12p40 and (e) IP-10. (f, g) C57/BL6 mice (n=5) received subcutaneous administration of 50 μg of OVA formulated with adjuvant or control at days 0 and 14. (f) Tetramer⁺ CD8 T cell responses were evaluated at day 24. (g) Mice were challenged intravenously at day 28 with LM-OVA and bacterial burden in spleens was evaluated on day 31; significance is relative to OVA, without adjuvant. All data are represented as mean±SEM; significance was calculated using ANOVA with Bonferroni correction; ns, not significant (P>0.05); *, P<0.05; **, P<0.01.

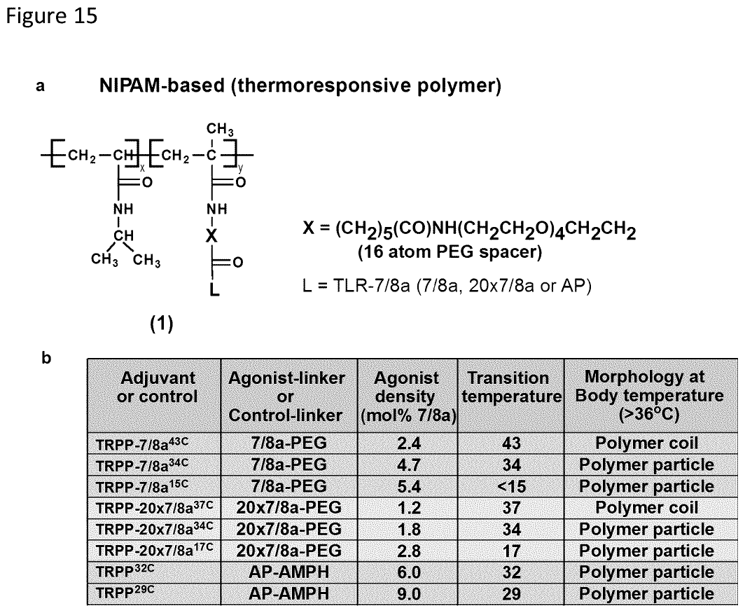

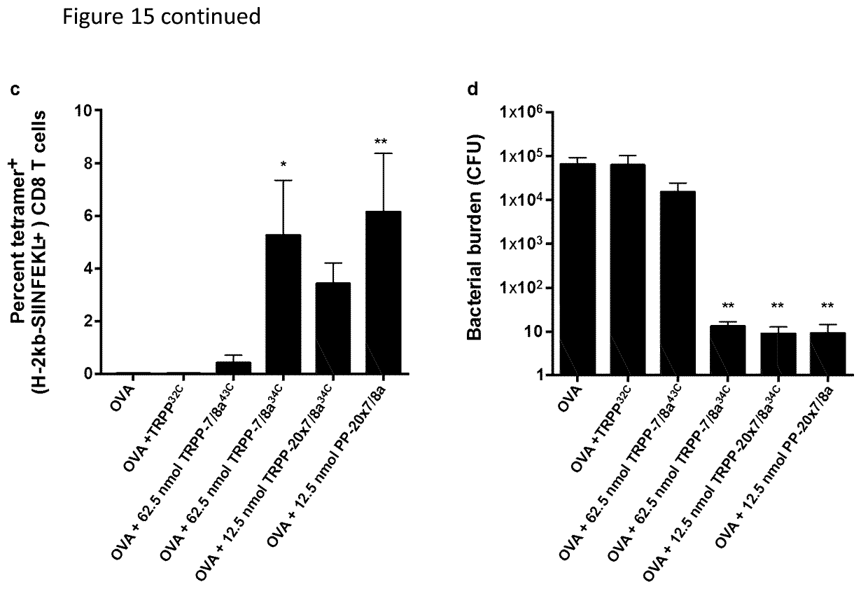

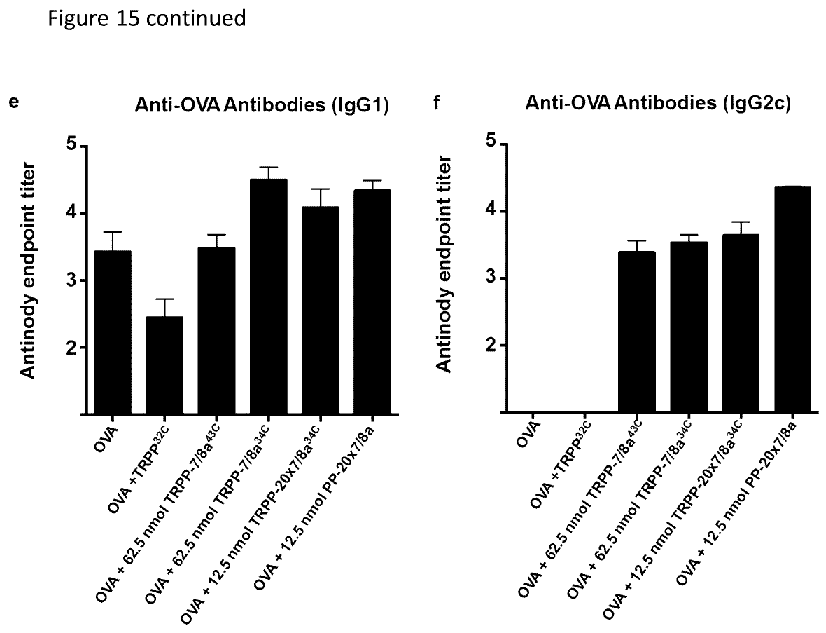

FIG. 15: (a) First-generation TRPP-7/8a are N-Isopropylacrylamide (NIPAM)-based copolymers. Note that the TLR-7/8a (7/8a or 20×7/8a) or a control ligand (AP) were attached to the NIPAM-based copolymers using a similar reaction scheme as described in supplementary FIG. 1 (see materials and methods). (b) A series of TRPP-7/8a were produced with increasing densities of either SM 7/8a, SM 20×7/8a or the control, AP-AMPH. Note that increasing densities of the hydrophobic ligands attached to the polymers leads to decreasing transition temperatures, the temperature at which particle formation occurs in aqueous solution. (c, d) TRPP-7/8a and controls were evaluated in a vaccination and challenge model using OVA. C57BL/6 mice (n=5) received 50 μg of OVA either alone or admixed with adjuvant that was administered subcutaneously in 50 μL of PBS at days 0 and 14. (c) At day 24, the proportion of tetramer⁺ CD8 T cells was evaluated from whole blood. (d) The capacity of the tetramer⁺ CD8 T cells to mediate protection was assessed by challenging the mice intravenously at day 28 with LM-OVA. Bacterial burdens were assessed in the spleen at day 31. (e, f, g) Serum was collected from vaccinated mice at day 28 and evaluated for (e) anti-OVA IgG1 and (f) IgG2c antibodies (geometric mean). Data are reported as mean±SEM; statistical significance is relative to OVA alone (ANOVA with Bonferroni correction); ns, not significant (P>0.05); *, P<0.05; **, P<0.01.

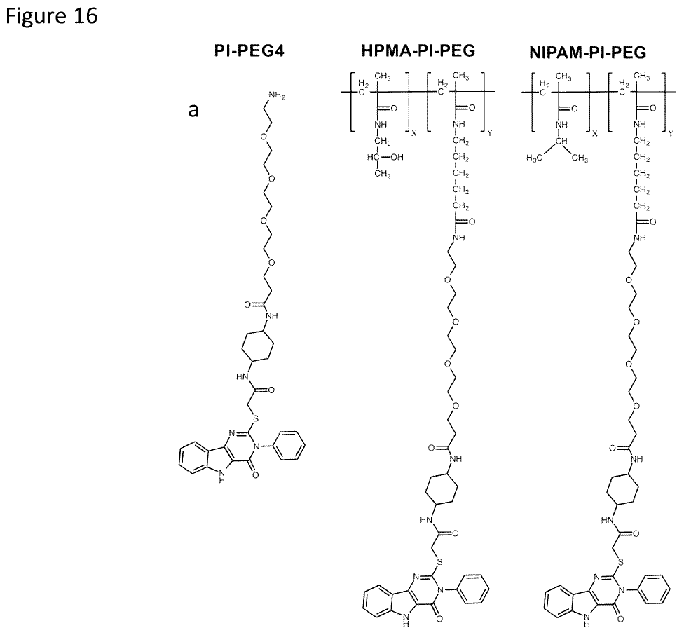

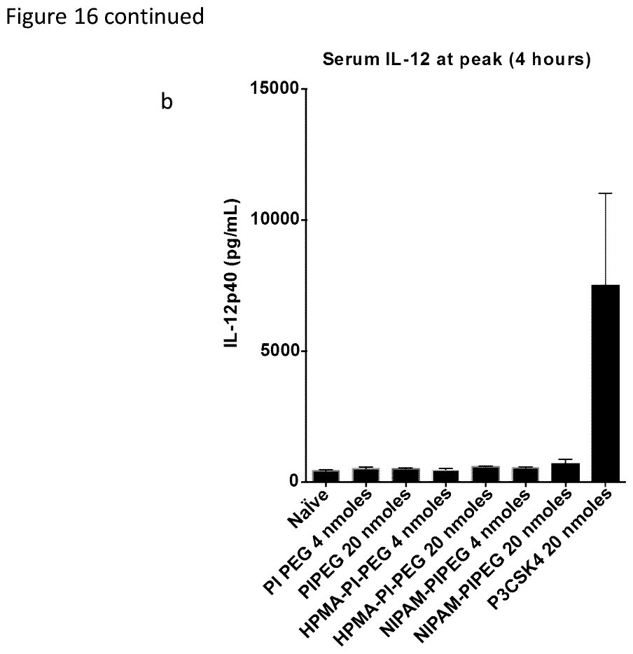

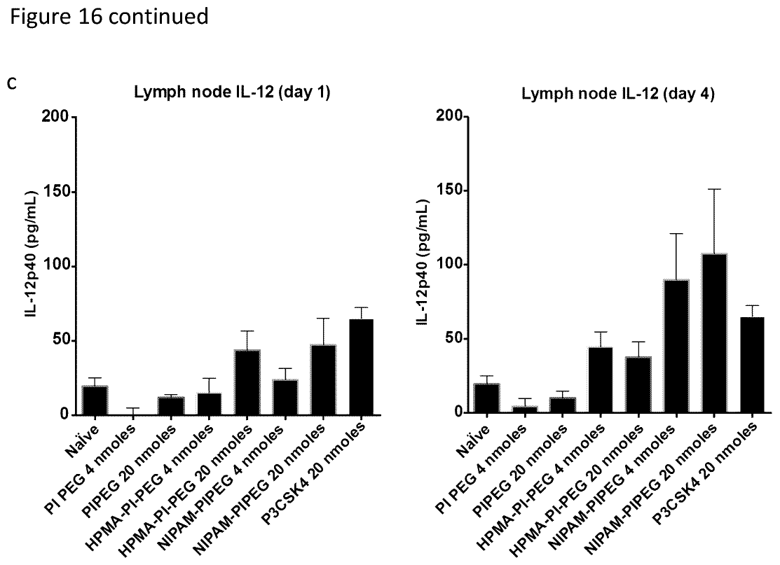

FIG. 16: Thermo-responsive polymer particle (PP) carriers of other PRRa enhance lymph node innate immune activity and reduce systemic toxicity. (a) HPMA and NIPAM-based polymer carriers of a pyrimidoindole-based TLR-4a (PP-PI). (b) The various different PP-PI conjugates or unconjugated TLRa were administered into the hind footpads of mice and evaluated for (b) serum and (c) lymph node IL-12p40 production (n=5). All data are represented as mean±SEM.

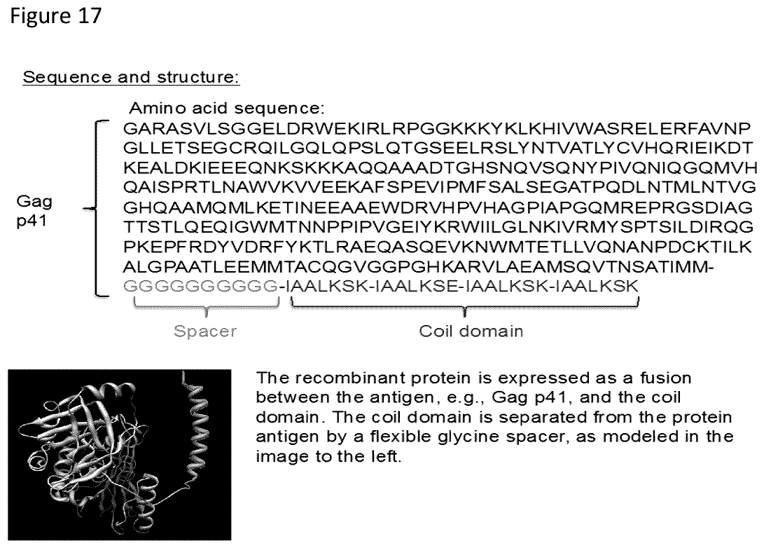

FIG. 17: HIV-Gag coil fusion protein used for site-selective attachment to thermo-responsive polymer adjuvants (TRPP-7/8a).

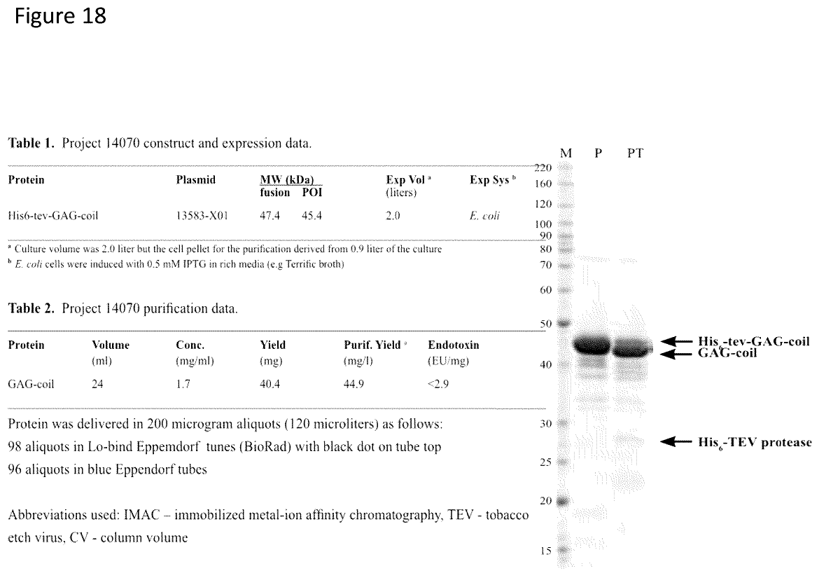

FIG. 18: Successful expression of the Gag-coil from bacteria.

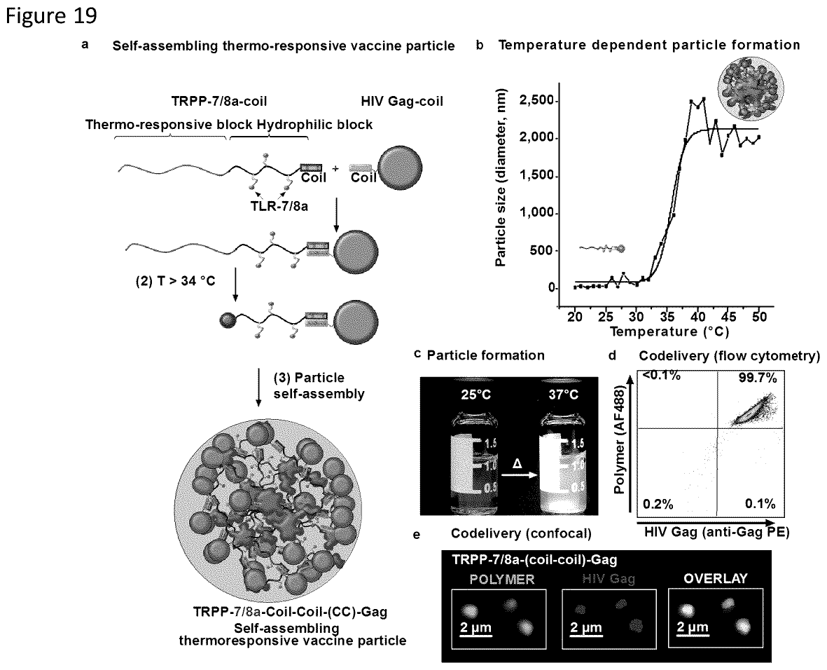

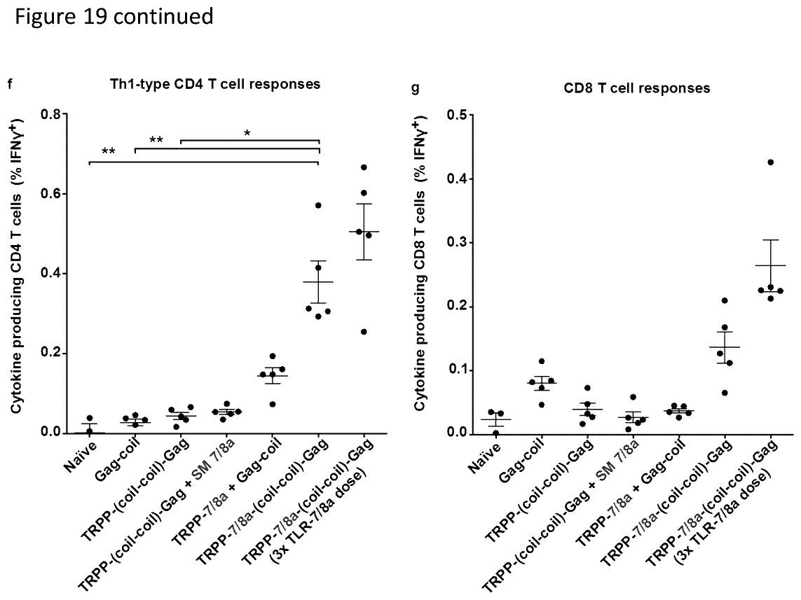

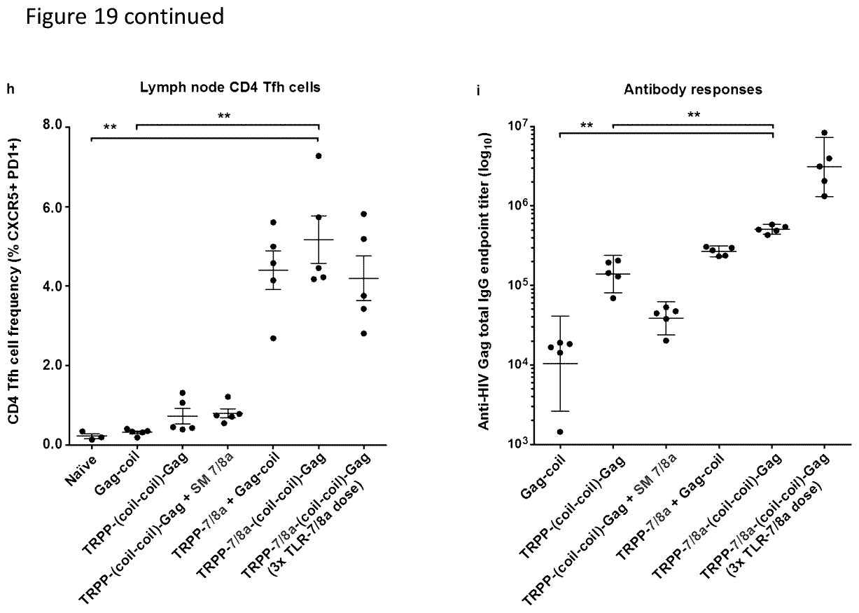

FIG. 19: Co-delivery of TLR-7/8a and HIV Gag-coil fusion protein antigen on a self-assembling thermo-responsive vaccine particle. (a) Cartoon schematic of a thermo-responsive Poly-7/8a (TRPP-7/8a) modified with a coil peptide that forms heterodimers with a recombinant HIV Gag-coil fusion protein. TRPP-7/8a-(coil-coil)-Gag complex formation occurs at room temperature and particle formation of the resulting complex occurs at temperatures greater than 33° C. (b) Temperature-dependent particle formation illustrated by dynamic light scattering. (c) Aqueous solutions of TRPP-7/8a-(coil-coil)-Gag at 25° C. and 37° C. (d, e) Co-localization of HIV Gag (labeled with anti-Gag PE) with TRPP-7/8a (labeled with carboxyrhodamine 110) was confirmed by (d) flow cytometry and (e) confocal microscopy. (f-i) BALB/c mice received subcutaneous administration of 50 μg of HIV-Gag coil formulated with either a control or TRPP-7/8a normalized for TLR-7/8a dose (1× dose=2.5 nmol, or 3× dose=7.5 nmol) at days 0 and 14. At day 28, DLN, spleen and serum from vaccinated mice were collected for analysis. Splenocytes were stimulated in vitro with an HIV Gag peptide pool. Antigen-specific IFNγ-producing (f) CD4 T cells (n=5) and (g) CD8 T cells (n=5) in the mixed splenocyte cultures, as well as (h) Tfh cells (n=5) in draining lymph nodes were quantified by flow cytometry. (i) Serum was evaluated for anti-HIV Gag total IgG antibody titers (n=5). In vivo studies are representative of two independent experiments. Data on linear axes are reported as mean±SEM. Data on log scale are reported as geometric mean with 95% CI. Comparison of multiple groups for statistical significance was determined using Kruskal-Wallis ANOVA with Dunn's post test; ns, not significant (P>0.05); *, P<0.05; **, P<0.01.

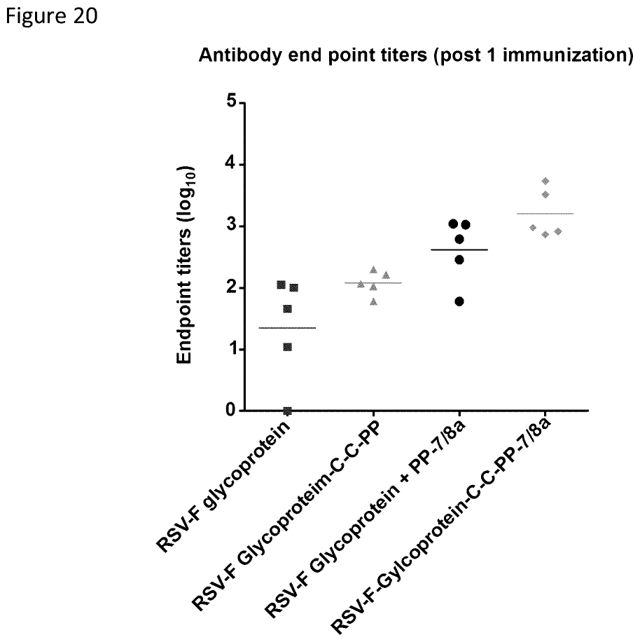

FIG. 20: Co-delivery of TLR-7/8a and RSV-F glycoprotein on a self-assembling thermo-responsive vaccine particle enhances antibody responses.

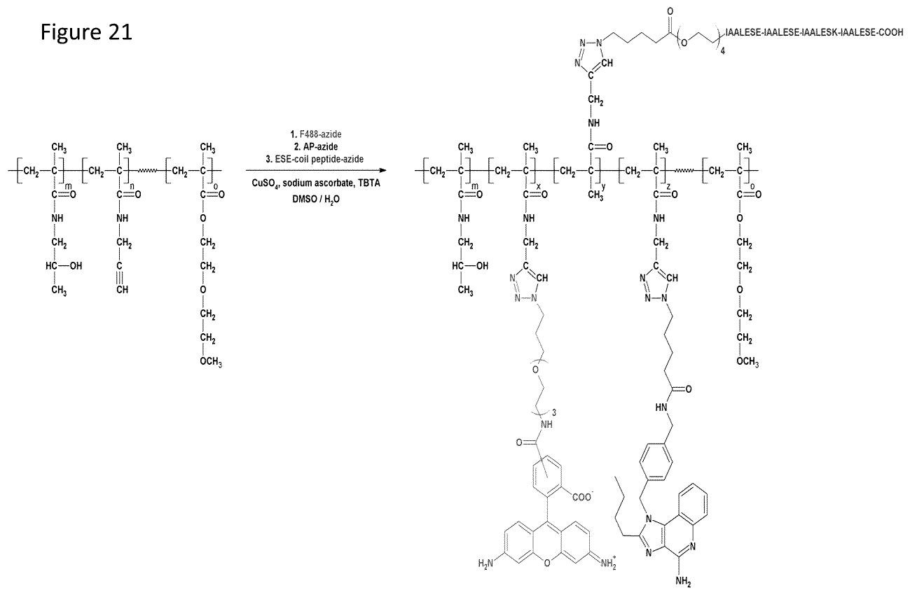

FIG. 21: Example adjuvant preparation scheme.

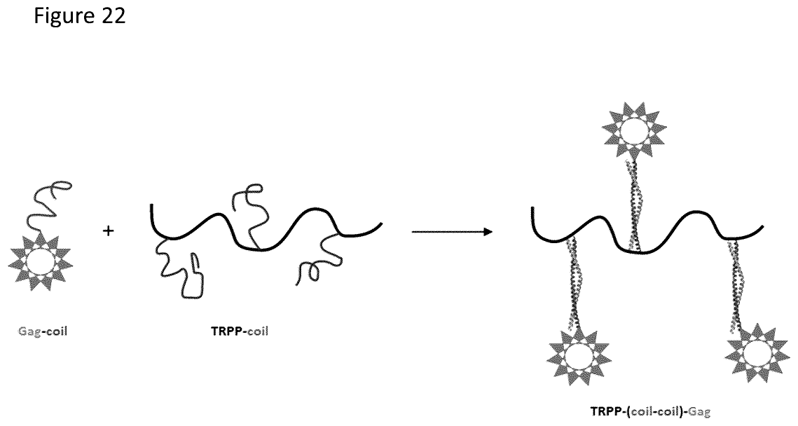

FIG. 22: Schematic diagram showing attachment of HIV Gag-KSK to fluorescently labelled TRPP-ESE conjugate via the coiled coil interaction

### INTRODUCTION

In reducing the invention to practice, it was evaluated how physicochemical parameters of TLRa delivery directly influence the magnitude and spatiotemporal characteristics of innate immune activation in vivo and how these responses translated to protective cellular immunity in the context of vaccination. Imidazoquinoline-based TLR-7/8a that bind to endosomally localized receptors within APCs were used as model adjuvants for these studies. Combined TLR-7/8 agonists (TLR-7/8a) have been shown to broadly activate multiple APC subsets in mice and humans and elicit a potent cytokine milieu (e.g., IL-12, type I Interferons) for generating cellular immunity. To modulate delivery, TLR-7/8a were linked to biocompatible polymer scaffolds in a combinatorial process that resulted in a diverse array of Polymer-TLR-7/8a conjugates (Poly-7/8a) that were screened in vivo. Properties that are important for activity were identified, including scaffold morphology, TLR-7/8a density (spacing of agonists on the scaffold) and linker group composition, and it was shown that particle formation is an important characteristic for enhancing the activity of Poly-7/8a. Biodistribution and kinetics studies together with cellular-level analysis of APC populations were used to mechanistically define how particle-forming Poly-7/8a enhance innate immune activation in lymph nodes by increasing local retention and promoting uptake by APCs. Increasing the density or potency of TLR-7/8a attached to the particle-forming Poly-7/8a, as well as the dose administered, increased the persistence of innate immune responses (>8 days), which we show is critical for inducing protective CD4 and CD8 T cell responses in two infectious challenge models of Leishmania major and Listeria monocytogenes, respectively. To extend these findings, thermo-responsive Poly-7/8a that exist as single water-soluble macromolecules during manufacturing and storage but undergo temperature-driven particle formation in vivo were developed to provide the benefits of soluble formulations in vitro during manufacturing and storage—high chemical definition and stability—with the improved activity of particulate adjuvants in vivo. To substantiate the observation that particle formation by the linear polymer carriers linked to PRRa was important for enhancing immunogenicity in vivo, additional TLRa and protein antigens were evaluated, including TLR-2/6 and TLR-4 agonists. Antigens L. major proteins (parasite) and peptide-based tumor neoantigens, as well as RSV-F glycoprotein and HIV Gag full-length viral protein antigens linked to the particle-forming polymer adjuvant compositions. The results presented herein will make clear the advantages of the invention over the prior art. The present invention (FIG. 1) deals with an adjuvant composition comprised of linear polymer chains that are covalently linked to adjuvant that undergo particle formation in aqueous conditions due to the hydrophobic characteristics of the attached PRR agonist or other ligand molecules. Alternatively, wherein the polymer is a thermo-responsive polymer, the adjuvant composition is comprised of thermo-responsive polymers linked to PRRa that exist as single water-soluble molecules in aqueous conditions during manufacturing and storage but that only undergo reversible particle formation at defined temperatures in vitro or at body temperature in vivo.

WO 2014/142653 A and CA 2627903 A describe vaccines comprised of particle matrices, wherein individual thermoresponsive polymer chains are cross-linked together and either entrap antigen/adjuvant or the antigen/adjuvant is covalently linked to the 3D particle matrix during the cross-linking step as in WO 2014/142653 A or is covalently attached to the surface of the particle as in CA 2627903 A. Thus, these patents describe particle vaccines comprised of cross-linked polymers that are fixed particles, rather than individual polymer chains that can reversibly form particles as in the present invention described herein. It is notable that there is no evidence to support the effects of such prior art systems on immunological activity. An advantage of the present invention is that using chemically defined single linear or branched polymers linked to PRRa addresses the limitations of preformed particles (poor chemical definition and long-term stability in aqueous conditions) and allows for control over precise loading (linkage) of PRRa and antigens on each polymer chain, which is not possible when modifying preformed particle such as the aforementioned prior art.

Another advantage of the present invention is that the temperature responsive polymers linked to PRRa exist as linear polymers but only form nanoparticles upon heating, whereas much of the referenced prior art relates to temperature-responsive particles that form amorphous gel matrices upon heating, which are distinct from the solution of defined spherical nanoparticles that define the present invention. For instance, US 2010/0028381 A, US 2013/0237561 A and the article by Shi et al (Shi, H. S. et al. Novel vaccine adjuvant LPS-Hydrogel for truncated basic fibroblast growth factor to induce antitumor immunity. Carbohydr Polym 89, 1101-1109 (2012)), describe the use of either pluronic, PEG-PLGA or PEG-poly(caprolactone) preformed particles that form amorphous gel matrices upon heating and can be used to physically entrap adjuvants, rather than physically linking PRRa directly to the polymer backbone as in the present invention. In contrast to these gels with non-linked adjuvant, the present invention described herein uses linear polymers covalently linked to PRRa that form spherical nanoparticles that are capable of targeting APCs in draining lymph nodes upon administration to a subject. Another advantage of the invention is that the single polymer chains linked to adjuvant that comprise the invention can be less than 10-20 nm in size and can be cost-effectively sterilely filtered, whereas the preformed particles described in the referenced prior are too large to use sterile filtration and require more costly methods of purification during production.

US 2012/0141409 A describes multivalent array of adjuvants on polymer chains that do not form particles. In contrast the present invention shows that polymer chains with multivalent arrayed adjuvants must form particles and have the adjuvant arrayed at a high density to promote high magnitude protective T cell responses.

Finally, Shakya et al (Shakya, A. K., Holmdahl, R., Nandakumar, K. S. & Kumar, A. Characterization of chemically defined poly-N-isopropylacrylamide based copolymeric adjuvants. Vaccine 31, 3519-3527 (2013)) describe the use of a thermo-responsive polymer to deliver a protein antigen to elicit antibodies but only associate antibody responses induced by the polymer with the chemical rather than physical (particle) nature of the vaccine formulation. Moreover, the authors did not evaluate the use of PRRa on polymers, as in the present invention.

The advantage of the present invention relates to the finding that linear polymers linked to PRRa require assembly into particles to optimize innate and adaptive immunity in vivo. An additional finding was that increasing the density of PRRa linked to the linear polymers results in enhanced magnitude and duration of innate immune activation that drives CD8 T cell responses. While much of the prior art relates to gels that physically entrap adjuvant to form immunogenic compositions, the data presented herein show importantly that spherical particles carrying PRRa can traffic to draining lymph nodes to target immune cells to enhance immunity, whereas the amophorous gel matrices described in previous reports are too large to traffic to lymph nodes.

### Results

### Combinatorial Synthesis and In Vivo Structure-Activity Relationship Studies Identify Parameters Important for Poly-7/8a Activity

An aim of this study was to define how properties of adjuvant delivery platforms influence innate immune activity in draining lymph nodes, the site of T cell priming following immunization. Immunologically inert HPMA-based polymers were chosen as scaffolds for initial studies for the delivery of TLR-7/8a since they are safe and effective delivery platforms for use in humans for other indications. Polymer-reactive TLR-7/8a were prepared according to previous reports (FIG. 2) and linked to reactive HPMA-based polymer carriers (FIG. 3) to produced polymer-TLR-7/8a conjugates (referred to herein as Poly-7/8a), which are protein-sized (˜30-50 kd) linear macromolecules with pendantly arrayed TLR-7/8a (FIGS. 3 and 4) . . . . It was hypothesized that certain properties, such as the density of agonists (mol % 7/8a, which relates to spacing of TLR-7/8a along the polymer), or chemical composition (hydrophobic/hydrophilic, length) of linkers anchoring the agonist to the polymer, may be important for activity. To evaluate these and other parameters, Poly-7/8a with varying physicochemical properties were generated by synthesizing various TLR-7/8a and control ligands (FIG. 5) with polymer carriers through combinatorial synthesis (FIG. 6) and then screened in vivo for the capacity to induce critical cytokines (I1-12, IP-10, IFNα and IFNγ) for driving cellular immune responses.

Comparing different Poly-7/8a normalized for TLR-7/8a dose, increasing agonist density significantly increased lymph node cytokine production (FIGS. 7 and 8). Poly-7/8a with the hydrophilic PEG linker induced the highest production of IL-12 in vivo and was used for the remainder of the experiments in this study. Importantly, enhanced in vivo activity by increasing densities of TLR-7/8a was associated with Poly-7/8a assembling into particles in aqueous conditions (FIG. 7b and FIG. 8). Accordingly, whereas Poly-7/8a with low to intermediate agonist densities (1-4 mol % 7/8a) adopt random coil confirmations, referred to as polymer coils (PC, ˜10 nm diameter), in aqueous conditions and induce no measurable cytokine production, Poly-7/8a with high agonist density (8-10 mol % 7/8a) assemble into submicron polymer particles (PP, ˜700 nm diameter) and induce significantly higher lymph node cytokine production as compared with the SM 7/8a and polymer controls (FIGS. 7b and 8). These structure activity studies suggest that either increasing densities of agonist on polymers, particle formation, or both, are critical to the in vivo cytokine responses by Poly-7/8a.

Previous studies report that particles alone can induce innate immune activation through the NLRP3 Inflammasome. However, cytokine responses by particle-forming Poly-7/8a with high agonist density (PP-7/8a^{10%}) were found to be dependent on TLR-7 but independent of Caspase 1/11 (FIG. 8f,g).

Particle Morphology Enhances Retention and APC Uptake Necessary for Persistent Innate Immune Activation that Drives T Cell Responses

The next studies assessed the in vivo mechanisms that account for how different physicochemical characteristics of TLR-7/8a delivery influence both innate and adaptive immune responses. First, the effect that different morphologies (small molecule, polymer coil and polymer particle) and densities of TLR-7/8a (Lo=3-4 and Hi=10 mol % 7/8a) have on biodistribution (FIG. 9a), kinetics (FIG. 9b) and cellular localization (FIG. 9c-f) were evaluated following subcutaneous administration of dye-labeled materials normalized for TLR-7/8a dose. While unformulated SM 7/8a distributed systemically and was undetectable after 1 day, all Poly-7/8a were retained locally and persisted in draining lymph nodes for up to 20 days, with particulate morphologies exhibiting the highest retention. Analysis of lymph node APC populations by flow cytometry (FIG. 9c) shows that all polymers independent of morphology or agonist density—are taken up by high proportions of total DCs (40-50%, FIG. 9d). However, the relative amount of material taken up by individual DCs was markedly higher for polymer particles as compared with polymer coils (FIG. 9e), which is particularly evident when comparing PP-7/8a^{Lo} with agonist density and dose matched PC-7/8a^{Lo} (FIG. 9f).

Particulate Poly-7/8a also led to significantly higher recruitment and activation of DCs in draining lymph nodes (FIG. 9g,h) as compared with all other groups. Relatively inefficient uptake of the polymer coil Poly-7/8a (PC-7/8a^{Lo}) by DCs was associated with limited DC recruitment, activation and cytokine production in lymph nodes (FIG. 9g-i). In contrast, the SM 7/8a was associated with splenic APC high levels of serum cytokines (FIG. 9j) but induced limited lymph node responses (FIG. 9g-i). Notably, increasing agonist density on particle forming Poly-7/8a led to more persistent cytokine production (FIG. 9i).

Different Poly-7/8a and controls were co-administered with a model antigen, ovalbumin (OVA). After two immunizations, the particulate Poly-7/8a with high agonist density (PP-7/8a^{Hi}) resulted in significantly higher CD8 T cell responses as compared with all other groups (FIG. 9k), and these responses were durable up to 10 weeks after vaccination (FIG. 9m). Notably, particulate Poly-7/8a with increasing agonist densities, potencies and doses was associated with increasing magnitude of CD8 T cell responses (FIG. 9l). Antibody responses induced by the different adjuvants co-administered with protein antigen was also assessed. The particle forming Poly-7/8a with high TLR-7/8a density was also associated with higher magnitude total anti-OVA IgG antibody titers as well as Th1-skewed antibody responses (FIG. 9n,o).

Together, these data suggest that particulate morphology and high agonist density, potency and dose are critical for promoting high magnitude and persistent innate immune activation necessary for generating T cell responses using polymers linked to PRR agonists.

### Persistent Local (Lymph Node) Activity by Poly-7/8a is Necessary and Sufficient for Inducing Protective Th1-Type CD4 and CD8 T Cell Responses

To benchmark innate and adaptive immune responses, the lead Poly-7/8a (PP-7/8a^{Hi}) was compared with two commercially available TLRa, the small molecule TLR-7/8a, R848 (Resiquimod), and a TLR-9 agonist, CpG, which is especially potent in mice due to broad expression of TLR-9 across murine APC subsets. Whereas R848 only induced systemic (sera) cytokines (FIG. 10a,b), the Poly-7/8a, PP-7/8^{Hi}, induced significantly higher levels of local cytokines but low systemic cytokine responses. In contrast, CpG induced high levels of both local and systemic cytokine production (FIG. 10a,b). Systemic inflammation induced by R848 and CpG was closely associated with transient decreases in mouse body weight (FIG. 10c), a finding that was observed for other systemic, but not locally acting, adjuvants.

Persistent local activity was found to be critical to the capacity of the adjuvants to induce protective CD8 T cell responses when co-administered with the protein antigens OVA and SIV Gag (FIG. 10). Poly-7/8a and CpG, which induce persistent local innate immune activity, both elicit CD8 T cell responses that are of sufficient magnitude to protect against infectious challenge with Listeria monocytogenes expressing OVA (LM-OVA). In contrast, R848, which induces high levels of transient systemic cytokine production, but no local activity, provided no improvement in CD8 T cell responses or protection as compared with protein immunization alone.

Vaccines against parasitic and mycobacterial infections will likely need to elicit potent and durable Tₕ1-type CD4 cells. The capacity of Poly-7/8a to induce such responses was assessed in the mouse model of Leishmania major, which requires Tₕ1 CD4 cells to clear the parasite from infected cells. Mice were immunized with MML, a protein derived from L. major, either alone or with adjuvant. Poly-7/8a and CpG induced comparable magnitudes and qualities of Tₕ1-type CD4 cells, while responses to MML co-administered with either the SM 7/8a or polymer controls were equivalent to MML administered alone (FIG. 11a). Following intradermal challenge with an infectious dose of L major, mice immunized with MML+Poly-7/8a or CpG had significant reductions in ear lesion size and more rapid resolution of the infection. These results show that the particulate Poly-7/8a can induce protective Tₕ1 CD4 cell responses (FIG. 11b).

Recent clinical trials data has emerged suggesting that the ability of checkpoint inhibitors (e.g., anti-PD1, anti-CTLA4 antibodies) to mediate tumor regression in patients in part depends on the capacity of these immunotherapies to activate otherwise quiescent CD8 T cell responses against mutated self-proteins expressed by the cancer, referred to as neoantigens. One means of enhancing neoantigen-specific CD8 T cell responses is through vaccination. As a proof-of-concept for the capacity of the present invention to induce CD8 T cell responses against tumor neoantigens, the particle-forming Poly-7/8a was co-administered with a model tumor neoantigen, Reps1, recently described by Yadav et al and derived from murine melanoma (Yadav, M. et al. Predicting immunogenic tumour mutations by combining mass spectrometry and exome sequencing. Nature 515, 572-576 (2014).) The optimized particle-forming polymer (PP-7/8a) described herein induced higher magnitude Reps1-specific CD8 T cell responses as compared with CpG and pICLC (FIG. 12), indicating that the PP-7/8a is an effective adjuvant for inducing neoantigen-specific CD8 T cell responses.

### Polymer Carriers of Additional PRRa

Agonists of TLR-2/6 and TLR-4 were linked to linear polymers (FIG. 13) to extend the finding that linking PRRa to linear polymers that assemble into particles is an effective means of enhancing local innate immune activity while reducing systemic toxicity. The data show that linking TLR-2/6 and TLR-4a to linear polymer carriers that assemble into particles is an effective means of enhancing DC activation and cytokine production (FIG. 13 b,c), while reducing systemic cytokines and morbidity (loss of body weight) associated with the free, un-linked TLR-2/6 and TLR-4 agonists (FIG. 13 d,e).

In Vivo Particle Formation with Thermo-Responsive Poly-7/8a Enhances Local Innate Immune Activation and Protective Cellular Immunity

Having demonstrated the requirement of particle assembly for the in vivo activity of Poly-7/8a, but acknowledging the inherent challenges in the manufacturing and storage of particle-based adjuvants, thermo-responsive polymer particle (TRPP)-7/8a conjugates were developed that exist as water soluble random coil-forming macromolecules during manufacturing and storage (T<30° C.) but undergo particle assembly in vivo (T<36° C.), above a thermodynamically-defined transition temperature (FIG. 14). The transition temperature of TRPP-7/8a was tuned by modulating the density and or hydrophilic/hydrophobic character of ligands attached to the polymer backbones (FIG. 14c and FIG. 15a,b), allowing the production of TRPP-7/8a that form particles below or above body temperature (FIG. 14 and FIG. 15). Consistent with earlier findings, only TRPP-7/8a capable of forming particles in vivo lead to persistent and high levels of local cytokines (FIG. 14d,e) that are sufficient for generating CD8 T cell responses that mediate protection (FIG. 14f,g and FIG. 15c,d) and enhance antibody responses (FIG. 15e,f). These results substantiate the importance of particulate adjuvants for inducing protective immune responses and show that in situ particle formation using thermo-responsive carriers can be a suitable alternative to using preformed particles. These results were extended to the delivery of a TLR-4a, showing that the thermo-responsive polymer can be used to potentate activity of additional TLRa (FIG. 16).

Finally, steps were taken to further refine the structure of TRPP-7/8a to promote biodegradability and improve generalizability of the approach. First, as bioaccumulation of polymers is a potential safety concern, a di-block copolymer with ester side chains was used to promote degradation of the particles to individual polymer chains that can be excreted by the kidneys. Secondly, prior studies have shown that synchronous delivery of protein antigen with innate immune stimulation is a highly efficient approach for optimizing T cell priming, a TRPP-7/8a was generated with coil peptides that provide a generalizable strategy for site-specially attaching antigen-coil fusion proteins to the polymer carriers through coiled-coil interactions. To demonstrate the utility of this approach, a recombinant HIV Gag-coil fusion protein (FIGS. 17-19) was site-specifically linked to a TRPP-7/8a through self-assembly using peptide-based coiled-coil interaction (FIG. 19). Mixing aqueous solutions of the HIV Gag-coil protein with a TRPP-7/8a modified with a complementary coil peptide results in self-assembly of a TRPP-7/8a-(coil-coil)-Gag complex that undergoes particle formation (FIG. 19a-c) at temperatures greater than 34° C. and ensures co-delivery of Gag with TRPP-7/8a (FIG. 19d,e). Co-delivery of Gag with TRPP-7/8a (TRPP-7/8a-(coil-coil)-Gag) resulted in enhanced T cell and antibody responses (FIG. 19 f-i).

To further demonstrate the generalizability of the thermo-responsive polymer platform for linking antigen and PRRa, an RSV-F trimeric glycoprotein was delivered on the TRPP-7/8a described above and the data shows that this approach is effective for inducing high titer antibody responses after a single vaccine administration.

### DISCUSSION

By accounting for biodistribution, kinetics and cellular localization, we establish how physicochemical parameters of PRRa delivery directly influence the location, magnitude and duration of innate immune activation in vivo. These studies established that polymers linked to high densities and potencies of PRRa and that assembled into particles are critical for inducing high magnitude and persistent (>8 days) innate immune activation in lymph nodes that is necessary for eliciting protective CD4 and CD8 T cell responses and high magnitude antibody responses.

Biodistribution is the most important factor dictating the balance between local (lymph node) activity and systemic inflammation. Whereas small molecule PRRa distributed systemically and resulted in high levels of transient (<24 h) systemic inflammation, polymeric particle carriers of PRRa were retained locally for 2-4 weeks and induced persistent local innate immune activation. Indeed, earlier studies have shown improved activity of various TLRa delivered on macromolecular or particulate carriers, or even after formulating the TLRa within particles. Taken together, these data suggest that improved activity by macromolecular and particulate delivery systems may be in part due to increased local retention. Local retention is critical to adjuvant activity but not sufficient. Despite improved retention by all polymer-based macromolecular carriers of PRRa, it was observed that only the particle forming polymer-PRRa conjugates are taken up efficiently by APCs and induce innate immune responses in lymph nodes.

Earlier studies have reported that persistent innate immune responses are important for inducing cellular immunity. In this study, additional clarification was provided by defining that innate immune activation >8 days in lymph nodes is critical for optimizing protective CD4 and CD8 T cell responses. Additionally, in contrast to earlier reports, it was observed that systemic cytokines are dispensable to CD8 T cell priming and expansion. Observations that lymph node cytokines, but not systemic cytokine production, are important for inducing CD8 T cell responses may provide clarity to what previous reports have referred to as the “temporal conundrum” regarding the discordance between when systemic cytokines and CD8 T cell responses peak, 2-6 hours and ˜7-10 days after vaccination, respectively.

### Example Polymers

The following examples provide details of polymers, agonists and linkers, which may be used in the invention individually or in the combinations provided (i.e. each linkage, functional group, or polymer described below may be used with any other linkage, functional group or polymer where appropriate). The skilled person will understand that alternative linkages and functional groups may be used in each example.

### Examples of Statistical-Copolymers

Copolymers of thermoresponsive monomers were prepared as previously described^{2, 3}. This includes polymers comprised of the above-mentioned thermoresponsive macromolecules-forming monomeric units (NIPAAm, NIPMAm, etc.) and methacrylate or methacrylamide-based monomeric units bearing the functional groups (FGs) attached to the methacryloyl moiety directly or through various spacers (SPs). The FGs include amino groups; azide group-reactive propargyl (Pg) and dibenzocyclooctyne groups (DBCO); alkyne group-reactive azide groups; thiol group reactive pyridyl disulfide (PDS) and maleimide (MI) groups; carbonyl-group reactive monohydrazide and aminooxy groups; and amino-group reactive N-succinimidyl ester (OSu), pentafluorophenyl (PFP) and carboxythiazolidin-2-thione (TT) groups. The SPs include aminoacyls (e.g. glycyl, β-alanyl, 6-aminohexanoyl, 4-aminobenzoyl, etc.), diamines (ethylenediamine, 1,3-propylenediamine, 1,6-diaminohexane, etc.) or oligo (ethylene glycol)-based derivatives comprising from 4 to 24 ethylene glycol units.

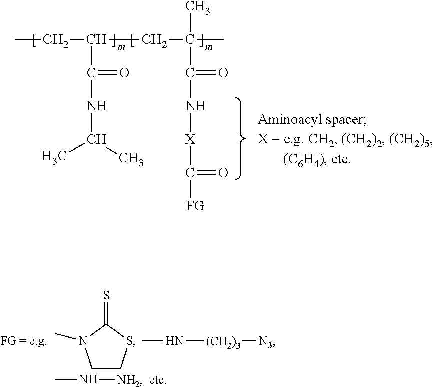

Formula 1: Example of statistical copolymer consisting of NIPAM monomeric units and methacrylamide-based monomeric units bearing the functional groups (FGs) attached to the methacryloyl moiety through the aminoacyl spacers.

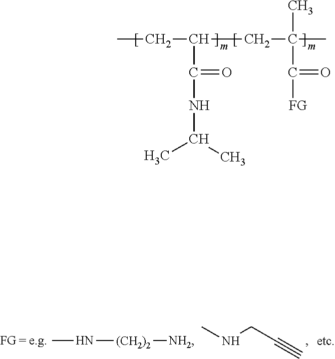

Formula 2: Example of statistical copolymer consisting of NIPAM monomeric units and methacrylamide-based monomeric units bearing the functional groups (FGs) directly attached to the methacryloyl moiety.

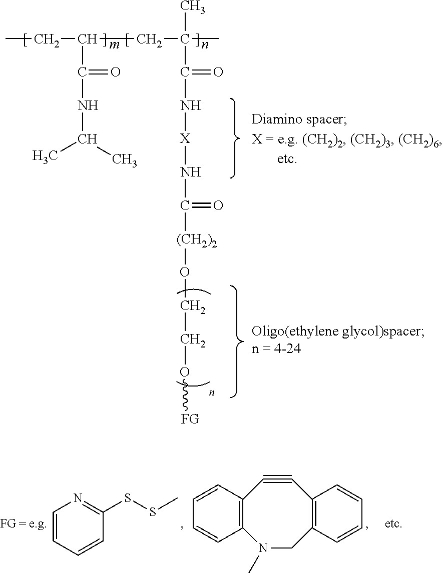

Formula 3: Example of statistical copolymer consisting of NIPAM monomeric units and methacrylamide-based monomeric units bearing the functional groups (FGs) attached to the methacryloyl moiety through the diamino and oligo (ethylene glycol spacers.

### Examples of Block-Copolymers

This includes A-B type of amphiphilic copolymers comprised of two blocks of polymers, where the first block is composed of macromolecules with hydrophilic character and the adjacent one is composed of macromolecules exhibiting the thermoresponsive properties, as described above. The hydrophilic block includes but is not limited to polymers and statistical copolymers comprised of dominant monomer unit N-(2-hydroxypropyl) methacrylamide (HPMA) and the all above mentioned comonomer units based on methacrylates or methacrylamides bearing the functional groups (FGs) attached to the methacryloyl moiety directly or through the various spacers (SPs). The thermo-responsive block includes polymers and statistical copolymers comprised of dominant thermo-responsive macromolecules-forming monomer units (see above) and (meth)acrylate or (meth)acrylamide-based monomeric units bearing the functional groups (FGs) attached to the methacryloyl moiety directly or through the various spacers (SPs).

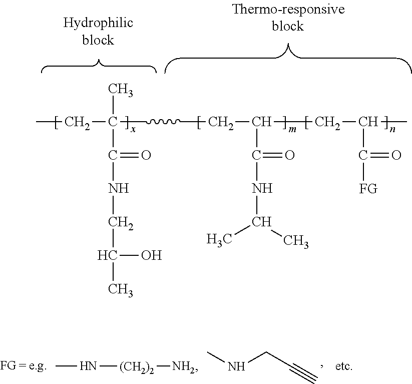

Formula 4: Example of A-B type diblock copolymer consisting of PHPMA hydrophilic block and PNIPAAm-based thermo-responsive block. The thermo-responsive block is composed of major NIPAM monomeric units and minor acrylamide-based comonomeric units bearing the functional groups (FGs) directly attached to the methacryloyl moiety.

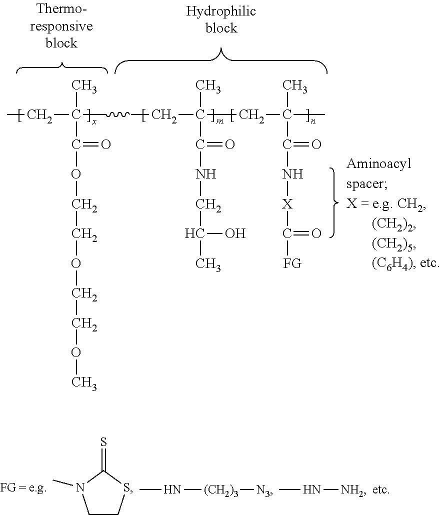

Formula 5: Example of A-B type diblock copolymer consisting of PDEGMA thermo-responsive block and PHPMA-based hydrophilic block. The hydrophilic block is composed of major HPMA monomeric units and minor methacrylamide-based monomeric units bearing the functional groups (FGs) attached to the methacryloyl moiety through the aminoacyl spacers.

### Examples of Graft-Copolymers

This includes statistical copolymers and/or A-B type diblock copolymers (see above), where the parts of the FGs in the side chains of the polymers are grafted to a protein molecule.

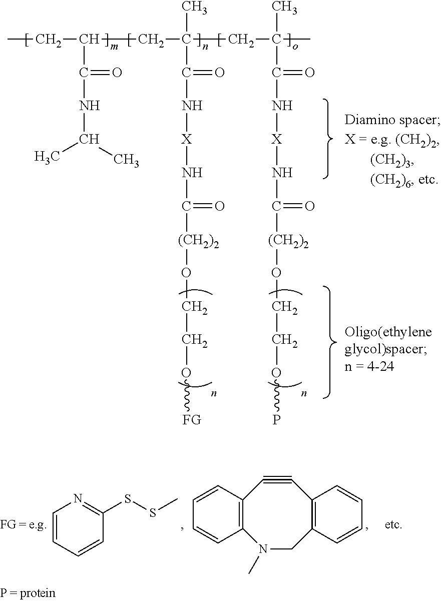

Formula 6: Example of NIPAM-based statistical copolymer grafted with a protein. The main polymer chain on to witch the protein is grafted is composed of major NIPAM monomeric units and minor methacrylamide-based comonomeric units bearing the functional groups (FGs) attached to the methacryloyl moiety through the diamino and oligo (ethylene glycol) spacers.

### Immune Potentiators (Adjuvants)

Immune potentiators can be any one of a broad and diverse class of synthetic or naturally occurring compounds that are recognized by pattern recognitions receptors (PRRs). The immune potentiator is attached to the thermoresponsive polymer carrier (described below). Examples of immune potentiators include the following PRR agonists:
a) Toll-like receptor (TLR) agonists: this includes but is not limited to TLR-1/2/6 agonists (e.g., lipopeptides and glycolipids); TLR-3 agonists (e.g., dsRNA and nucleotide base analogs), TLR-4 (e.g., lipopolysaccharide (LPS) and derivatives); TLR-5 (Flagellin); TLR-7/8 agonists (e.g., ssRNA and nucleotide base analogs); TLR-9 agonists (e.g., unmethylated CpG)

### Conjugatable TLR-7/8 Agonists

Several conjugatable TLR-7/8a that are suitable for attachment to thermoresponsive polymers are described in the literature⁴⁻⁷. Examples of conjugatable TLR-7/8a that were attached to the polymer carriers are shown:

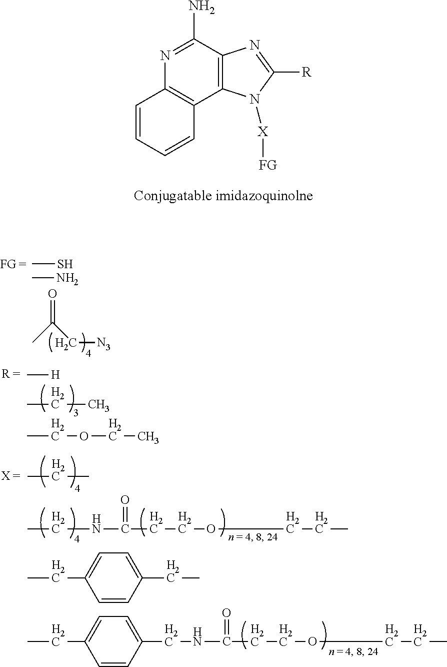

Formula 7: Conjugatable TLR-7/8 agonists. The structure in the top left is a generic conjugatable imidazoquinoline-based combined TLR-7 and TLR-8 agonist. Note that the R group can be changed to modulate specificity for either TLR-7 or TLR-8. X is the cross-linker and was prepared as a short butyl group or a xylene group with or without a PEG spacer. FG is the functional group that allows for attachment to the polymer chain using either a thiol, primary amine or azide group.

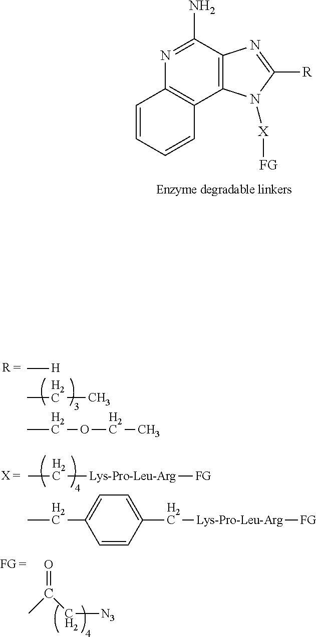

Formula 8: Conjugatable TLR-7/8 agonists with enzyme degradable linkers. Several TLR-7/8a were prepared with short tetrapeptides that are recognized and cleaved by protease (cathepsins). Note that the functional group on these peptides is an azide that permits selective attachment to polymer carriers using “click chemistry.”

### Conjugatable TLR-1/2/6 Agonists

Conjugatable derivatives of Pam2cys and Pam3cys were prepared from commercially available precursors as previously described⁸⁻¹⁰.

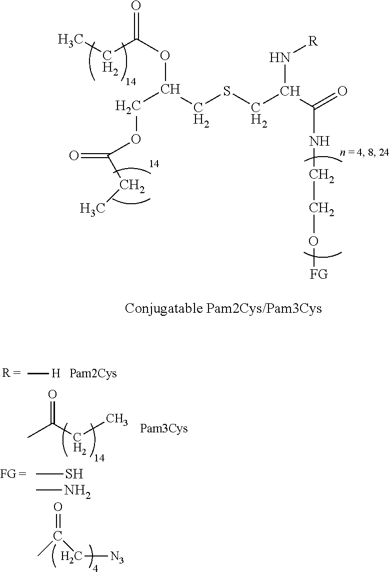

Formula 9: Conjugatable TLR-1/2/6 agonists. The structure in the top left is a generic conjugatable derivative of Pam2Cys (R═H) or Pam3Cys (R=palmitic acid). Note that the R group can be changed to modulate specificity for either TLR-1/2 or TLR-2/6. X is the cross-linker and was prepared as a PEG spacer. FG is the functional group that allows for attachment to the polymer chain using a thiol, primary amine or azide group.
b) NOD-like receptors (NLR) agonists: this includes but is not limited to peptidogylcans and structural motifs from bacteria (e.g., meso-diaminopimelic acid and muramyl dipeptide)
c) Agonists of C-type lectin receptors (CLRs), which include various mono, di, tri and polymeric sugars that can be linear or radially branched (e.g., mannose, Lewis-X trisaccharides, etc.)

### Conjugatable C-Type Lectin Receptors

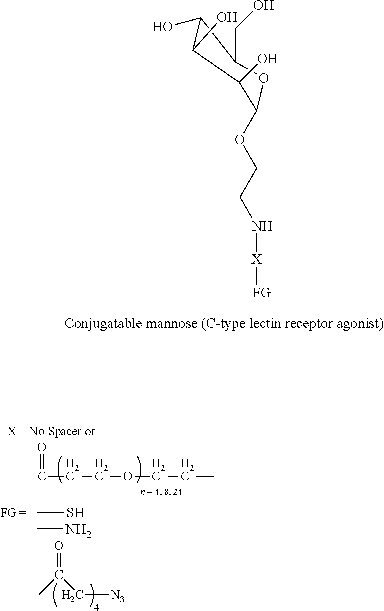

Formula 10: Conjugatable mannose derivatives. The structure in the top left is a generic mannose molecule. X is the cross-linker and was prepared as a PEG spacer. FG is the functional group that allows for attachment to the polymer chain using a thiol, primary amine or azide group.
d) Agonists of STING (e.g., cyclic dinucleotides)
1. Hruby, M. et al. New bioerodable thermoresponsive polymers for possible radiotherapeutic applications. Journal of controlled release: official journal of the Controlled Release Society 119, 25-33 (2007).
2. Subr, V. & Ulbrich, K. Synthesis and properties of new N-(2-hydroxypropyl)-methacrylamide copolymers containing thiazolidine-2-thione reactive groups. React Funct Polym 66, 1525-1538 (2006).
3. Nanba, R. J., Iizuka, Takao (JP), Ishii, Takeo (JP) (TERUMO CORP (JP), 1999).
4. Russo, C. et al. Small molecule Toll-like receptor 7 agonists localize to the MHC class II loading compartment of human plasmacytoid dendritic cells. Blood 117, 5683-5691 (2011).
5. Shukla, N. M., Malladi, S. S., Mutz, C. A., Balakrishna, R. & David, S. A. Structure-activity relationships in human toll-like receptor 7-active imidazoquinoline analogues. J Med Chem 53, 4450-4465 (2010).
6. Shukla, N. M. et al. Syntheses of fluorescent imidazoquinoline conjugates as probes of Toll-like receptor 7. Bioorg Med Chem Lett 20, 6384-6386 (2010).
7. Khan, S. et al. Chirality of TLR-2 ligand Pam3CysSK4 in fully synthetic peptide conjugates critically influences the induction of specific CD8+ T-cells. Mol Immunol 46, 1084-1091 (2009).
8. Khan, S. et al. Distinct uptake mechanisms but similar intracellular processing of two different toll-like receptor ligand-peptide conjugates in dendritic cells. J Biol Chem 282, 21145-21159 (2007).
9. Jackson, D. C. et al. A totally synthetic vaccine of generic structure that targets Toll-like receptor 2 on dendritic cells and promotes antibody or cytotoxic T cell responses. Proc Natl Acad Sci USA 101, 15440-15445 (2004).

### Examples of Protein or Peptide Antigens for Specific Disease Indications

### Cancer Peptide Antigen Attached to a Polymer Scaffold Through “Click Chemistry”

Peptide-based cancer antigens represent subunits of mutated forms of normal host proteins. Peptides such as NY-ESO from testicular cancer and NA17 from melanoma can induce responses in the general population; though, high throughput proteomics technology can be used to identify cancer antigens (e.g., peptides) that are unique to individual patients. Regardless of the source or exact structure of the antigen, peptides can be produced through solid-phase peptide synthesis that contain azide or alkyne “clickable” functional groups that allows for their attachment to polymer scaffolds using click chemistry.

The following tumor antigen, Na17 was produced with an N-terminal azide that allowed for coupling to the polymer scaffolds as previously described^{11, 12}.

### Example of Azido-(Cancer)-Peptide Produced by Solid-Phase Peptide Syntheis

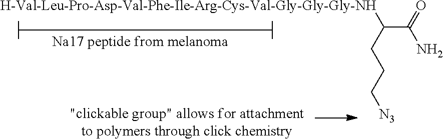

Recombinant Protein Antigens Fused with Polypeptide Domains (e.g., Coil Peptides) that Permit Site-Specific Attachment to Polymer Scaffolds

Protein antigens are typically larger than 100 amino acids and require complicated post-translational modification steps that require their production using in vitro expression systems. As such, in some circumstances it may not be easy to chemically incorporate “clickable”/bio-orthogonal groups, which allow for site-specific attachment into proteins. Instead, recombinant technologies can be used express antigens as fusion proteins with coil domains¹³, split inteins¹⁴ and Spy tags¹⁵ that permit site-selective docking to polymeric platforms.

### Example: HIV Gag Protein Produced as Protein-Coil Fusion to Attach to Polymers as Previously Described¹³

With reference to FIG. 20, HIV-Gag coil fusion protein was produced in yeast. The data shown in FIG. 21 demonstrates successful expression of the Gag-coil fusion protein for attachment to polymers. FIG. 22 shows a schematic representation of the incorporation of protein antigen (HIV-Gag) coil protein into a thermo-responsive polymer. FIG. 23 details the coil-coil interactions.

### REFERENCES

1. Shi, H. S. et al. Novel vaccine adjuvant LPS-Hydrogel for truncated basic fibroblast growth factor to induce antitumor immunity. Carbohydr Polym 89, 1101-1109 (2012).
2. Hruby, M. et al. New bioerodable thermoresponsive polymers for possible radiotherapeutic applications. Journal of controlled release: official journal of the Controlled Release Society 119, 25-33 (2007).
3. Subr. V. & Ulbrich, K. Synthesis and properties of new N-(2-hydroxypropyl)-methacrylamide copolymers containing thiazolidine-2-thione reactive groups. React Funct Polym 66, 1525-1538 (2006).
4. Nanba, R. J., Iizuka, Takao (JP), Ishii, Takeo (JP) (TERUMO CORP (JP), 1999).
5. Russo, C. et al. Small molecule Toll-like receptor 7 agonists localize to the MHC class II loading compartment of human plasmacytoid dendritic cells. Blood 117, 5683-5691 (2011).
6. Shukla, N. M., Malladi, S. S., Mutz, C. A., Balakrishna, R. & David, S. A. Structure-activity relationships in human toll-like receptor 7-active imidazoquinoline analogues. J Med Chem 53, 4450-4465 (2010).
7. Shukla, N. M. et al. Syntheses of fluorescent imidazoquinoline conjugates as probes of Toll-like receptor 7. Bioorg Med Chem Lett 20, 6384-6386 (2010).
8. Khan, S. et al. Chirality of TLR-2 ligand Pam3CysSK4 in fully synthetic peptide conjugates critically influences the induction of specific CD8+ T-cells. Mol Immunol 46, 1084-1091 (2009).
9. Khan, S. et al. Distinct uptake mechanisms but similar intracellular processing of two different toll-like receptor ligand-peptide conjugates in dendritic cells. J Biol Chem 282, 21145-21159 (2007).
10. Jackson, D. C. et al. A totally synthetic vaccine of generic structure that targets Toll-like receptor 2 on dendritic cells and promotes antibody or cytotoxic T cell responses. Proc Natl Acad Sci USA 101, 15440-15445 (2004).
11. Pola, R., Braunova, A., Laga, R., Pechar, M. & Ulbrich, K. Click chemistry as a powerful and chemoselective tool for the attachment of targeting ligands to polymer drug carriers. Polymer Chemistry 5, 1340-1350 (2014).
12. Jung, B. & Theato, P. in Bio-synthetic Polymer Conjugates, Vol. 253. (ed. H. Schlaad) 37-70 (Springer Berlin Heidelberg, 2013).
13. Pechar, M. & Pola, R. The coiled coil motif in polymer drug delivery systems. Biotechnology advances 31, 90-96 (2013).
14. Shah, N. H., Dann, G. P., Vila-Perello, M., Liu, Z. & Muir, T. W. Ultrafast protein splicing is common among cyanobacterial split inteins: implications for protein engineering. Journal of the American Chemical Society 134, 11338-11341 (2012).
15. Fierer. J. O., Veggiani, G. & Howarth, M. SpyLigase peptide-peptide ligation polymerizes affibodies to enhance magnetic cancer cell capture. Proc Natl Acad Sci USA 111, E1176-1181 (2014).

### Supplementary Materials and Methods

### Chemicals

All chemicals were purchased from Sigma-Aldrich (St. Louis, MO) as reagent grade or higher purity, unless stated otherwise. Ethoxyacetic acid was obtained from Alfa Acsar (Ward Hill, MA). Boc-15-amino-4,7,10,13-tetraoxapentadecanoic acid (PEG4) was purchased from EMD Millipore (Darmstadt, Germany). N-Boc-1,4-diaminobutane¹ and 2-Chloro-4,6-dimethoxy-1,3,5-triazine (CDMT)² were prepared as previously described. Green fluorescent reactive dyes Alexa Fluor® 488 carboxylic acid tetrafluorophenyl ester, Alexa Fluor® 488 cadaverine were purchased from Life Technologies (Carlsbad, CA) and Carboxyrhodamine 110 PEG3 azide was purchased from Alfa Aesar. Amine reactive infrared fluorescent reactive dye IRDye® 800CW NHS Ester was purchased from LI-COR (Lincoln, NE). Nucleophilic infrared fluorescent reactive dye, CruzFluor sm™ 8 amine, was purchased from Santa Cruz Biotechnology (Dallas, TX). Dibenzocyclooctyne (DBCO) modified PEG spacer (DBCO-PEG4-Amine) was purchased from Click Chemistry Tools (Scottsdale, AZ). Peptides were produced by solid phase peptide synthesis and were obtained from American Peptide Company (Vista, CA).

### Instrumentation for Synthesis, Purification and Chemical Characterization

Microwave irradiation was carried out in a CEM Discover Synthesizer with 150 watts max power. Flash column chromatography was performed on a Biotage SP4 Flash Purification system (Uppsala, Sweden) using Biotage® SNAP Cartridges and SNAP Samplet Cartridges with KP-Silica 60 mm. Analytical HPLC analyses were performed on an Agilent 1200 Series instrument equipped with multi-wavelength detectors using a Zorbax Stable Bond C-18 column (4.6×50 mm, 3.5 mm) with a flow rate of 0.5 mL/min or 1.0 mL/min. Solvent A was 0.05% trifluoroacetic acid (TFA) in water (H₂O), solvent B was 0.05% TFA in acetonitrile (ACN), and a linear gradient of 5% B to 95% B over 10 minutes was used. ESI or APCI mass spectrometry (MS) were performed on an LC/MSD TrapXCI Agilent Technologies instrument or on a 6130 Quadrupole LC/MS Agilent Technologies instrument equipped with a diode array detector. ¹H NMR spectra were recorded on a Varian spectrometer operating at 400 MHz. Ultraviolet-Visible (UV-Vis) light spectroscopy was performed on a Lambda25 UV/Vis system from PerkinElmer (Waltham, MA) and fluorescence spectroscopy was carried out on a PerkinElmer brand Fluorescence Spectrometer, model LS 55.

### Synthesis of Polymer Reactive Small Molecule TLR-7/8a

Synthesis of imidazoquinoline-based TLR-7/8a was based on previous reports^{3,7} and is described in more detail below.

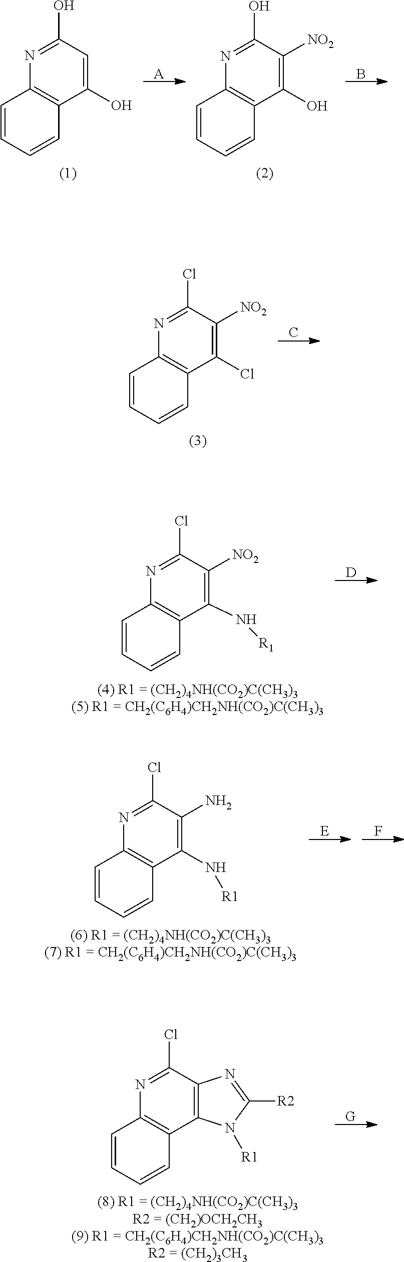

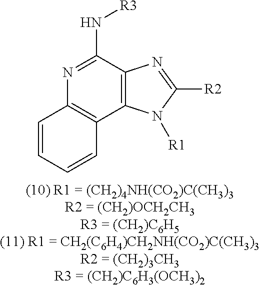

### Synthesis of imidazoquinoline-based TLR-7/8a: (A) HNO₃, heat; (B) PhPOCl₂, heat; (C) NH₂R₁, Et₃N, heat; (D) 10% Pt/c, H₂(g) 55 PSI, Ethyl acetate; (E) R₂COOH, CDMT, NMM, EtOAc; (F) CaO, heat, MeOH; (G) NH₂R₃, Et₃N, MeOH, heat; (H) H₂SO₄, heat

(4) The synthesis of tert-butyl (4-((2-chloro-3-nitroquinolin-4-yl)amino)butyl) carbamate was carried out as previously described^{3. 1}H NMR (400 MHZ, CDCl₃) δ 8.11 (d, J=7.6 Hz, 1H), 7.91 (dd, J=8.4, 1 Hz, 1H), 7.74 (m, 1H) 7.52 (m, 1H), 6.40 (br s, 1H), 4.66 (br s, 1H), 3.48 (m, 2H), 3.20 (m, 2H), 1.80 (m, 2H), 1.65 (m, 2H), 1.47 (br s, 9H). MS (APCI) calculated for C₈H₂₃ClN₄O₄, m/z, 394.1, found 394.9 (M+H)⁺.

(5) The synthesis of tert-butyl (4-(((2-chloro-3-nitroquinolin-4-yl)amino)methyl)benzyl) carbamate was carried out as previously described^{6. 1}H NMR (400 MHZ, DMSO-d6) δ 8.51 (d, J=8.5 Hz, 1H), 8.46 (t, J=6.4 Hz, 1H), 7.88-7.78 (m, 2H), 7.65 (dd, J=8.4, 5.5 Hz, 1H), 7.33 (t, J=6.2 Hz, 1H), 7.17 (q, J=8.2 Hz, 4H), 4.39 (d, J=6.2 Hz, 2H), 4.07 (d, J=6.2 Hz, 2H), 1.36 (s, 9H). MS (APCI) calculated for C₂₂H₂₃ClN₄O₄, m/z, 442.1, found 464.9 (M+Na)⁺.

(6) tert-butyl (4-((3-amino-2-chloroquinolin-4-yl)amino)butyl) carbamate. A 23 g solution of (5) and 230 mg of Na₂SO₄ in 200 mL of ethyl acetate was bubbled with Argon for 5 minutes to remove oxygen. To this solution, 230 mg of 10% Pt/c was added and the mixture was flushed with Argon for an additional 5 minutes and then pressurized with H₂(g) 55 mm Hg. The reaction mixture was agitated with a mechanical shaker. The reaction was considered complete (˜3 hours) once the pressure remained constant at a constant volume of H₂(g). The reaction mixture was filtered through celite and evaporated to dryness to obtain yellow oil. Trituration with 1:1 hexanes/ether yielded white crystals that were collected by filtration. Drying overnight under vacuum yielded 20.12 g (94.7% yield) of spectroscopically pure (>95% at 254 nm) white crystals. ¹H NMR (400 MHz, DMSO-d6) δ 8.03-7.95 (m, 1H), 7.70-7.61 (m, 1H), 7.44-7.34 (m, 2H), 6.73 (s, 1H), 5.14 (t, J=6.7 Hz, 1H), 5.00 (s, 2H), 3.19 (q, J=7.0 Hz, 2H), 2.87 (q, J=6.5 Hz, 2H), 1.55-1.34 (m, 4H), 1.33 (s, 9H). MS (APCI) calculated for C₁₈H₂₅ClN₄O₂, m/z, 364.2, found 365.2 (M+H)⁺.

(7) tert-butyl 4-(((3-amino-2-chloroquinolin-4-yl)amino) methyl)benzylcarbamate. The synthetic protocol is the same as for (6), except 5 g of (5) was used as the starting material. Product was spectroscopically pure (>95% at 254 nm) following passage through celite. Solvent was removed under vacuum and yielded 4.57 g (93% yield) of white crystals. ¹H NMR (400 MHZ, DMSO-d6) δ 8.00-7.93 (m, 1H), 7.63 (dd, J=8.0, 1.7 Hz, 1H), 7.35 (tt, J=6.9, 5.2 Hz, 2H), 7.31-7.25 (m, 3H), 7.11 (d, J=7.9 Hz, 2H), 5.79 (t, J=7.1 Hz, 1H), 5.04 (s, 2H), 4.40 (d, J=7.2 Hz, 2H), 4.04 (d, J=6.2 Hz, 2H), 1.36 (s, 9H). MS (APCI) calculated for C₂₂H₂₅ClN₄O₂, m/z, 412.2, found 413.2 (M+H)⁺.

(8) Tert-butyl (4-(4-chloro-2-(ethoxymethyl)-1H-imidazo[4,5-c]quinolin-1-yl)butyl) carbamate. To 2.5 mL of 2-ethoxyacetic acid (0.026 mol, 1.2 eq) in 150 mL of ethyl acetate were added 4.6 g (0.026 mol, 1.2 eq) CDMT, followed by dropwise addition of 6.0 mL (0.055 mol, 2.5 eq) of N-methylmorpholine (NMM). After 5 minutes, 8 g (0.022 mol, 1.0 eq) of (6) was added and the reaction was refluxed using an oil bath. A white precipitate was formed after several minutes corresponding to the NMM·Cl salt. After 16 hours, the reaction mixture was filtered and washed 3×150 mL with 1M HCl. The organic phase was dried with Na₂SO₄, filtered and evaporated to dryness. The resulting crude product was added to 20 mL of methanol with 800 mg (10% wt/wt) CaO and then microwaved at 100° C. for 3 hours. The CaO was removed by filtration and the resulting solution was evaporated to dryness to obtain an oily product that was purified by flash chromatography using a 0-6% methanol in DCM gradient, yielding 9.44 g of clear oil. Recrystallization from 5:1 hexane/ethyl acetate yielded 5.59 g (58.9% yield) of spectroscopically pure (>95% at 254 nm) white crystals. ¹H NMR (400 MHZ, DMSO-d6) δ 8.37-8.28 (m, 1H), 8.11-8.04 (m, 1H), 7.81-7.70 (m, 2H), 6.83-6.75 (m, 1H), 4.84 (s, 2H), 4.65 (t, J=7.9 Hz, 2H), 3.62-3.52 (m, 2H), 2.96 (q, J=6.4 Hz, 2H), 1.85 (t, J=7.9 Hz, 2H), 1.56 (t, J=7.7 Hz, 2H), 1.30 (s, 9H), 1.20-1.12 (m, 3H). MS (APCI) calculated for C₂₂H₂₉ClN₄O₃ m/z 432.2, found 433.2 (M+H)⁺.

(9) Tert-butyl 4-((2-butyl-4-chloro-1H-imidazo[4,5-c]quinolin-1-yl)methyl)benzylcarbamate. The synthetic protocol is the same as for (8), except 2 g of (7) was used as the starting material and pentanoic acid was used in place of 2-ethoxyacetic acid. Flash purification was not required, but the product was recrystallized from methanol to obtain 1.4 g (58% yield) of spectroscopically pure (>95% at 254 nm) yellow crystals. NMR (400 MHz, DMSO-d6) δ 8.08 (d, J=8.3 Hz, 1H), 8.02 (d, J=8.4 Hz, 1H), 7.63 (dd, J=8.2, 6.8 Hz, 1H), 7.50 (t, J=7.7 Hz, 1H), 7.30 (t, J=8 Hz, 1H), 7.15 (d, J=7.9 Hz, 2H), 7.01-6.94 (m, 2H), 5.94 (s, 2H), 4.04 (d, J=6.2 Hz, 2H), 2.96 (t, J=7.7 Hz, 2H), 1.73 (q, J=7.6 Hz, 2H), 1.38 (q, J=7.4 Hz, 2H), 1.33 (s, 9H), 0.86 (t, J=7.3 Hz, 3H). MS (APCI) calculated for C₂₇H₃₁ClN₄O₂ m/z 478.2, found 479.2 (M+H)⁺.

(10) Tert-butyl (4-(4-(benzylamino)-2-(ethoxymethyl)-1H-imidazo[4,5-c]quinolin-1-yl)butyl) carbamate. 6.5 g of (8) (0.015 mol, 1 eq) was added to 16 mL of benzylamine (0.15 mol, 10 eq) and reacted for 6 hours at 110° C. in a microwave apparatus (CEM Discover Synthesizer). After completion, the reaction mixture was cooled to room temperature and then added to 100 mL of DCM and washed 4×100 mL with 1 M HCl. The resulting yellow oil was recrystallized from 4:1 hexane/ethyl acetate to obtain 7.3 g (97.1%) of spectroscopically pure (>95% at 254 nm) white crystals. ¹H NMR (400 MHZ, DMSO-d6) δ 7.99 (d, J=8.0 Hz, 1H), 7.66-7.55 (m, 2H), 7.41 (d, J=7.3 Hz, 3H), 7.25 (td, J=7.5, 5.6 Hz, 3H), 7.20-7.12 (m, 1H), 6.80 (t, J=5.7 Hz, 1H), 4.79-4.72 (m, 4H), 4.53 (t, J=7.8 Hz, 2H), 3.54 (q, J=7.0 Hz, 2H), 2.95 (q, J=6.5 Hz, 2H), 1.85 (m, 2H), 1.54 (t, J=7.7 Hz, 2H), 1.31 (s, 9H), 1.15 (t, J=7.0 Hz, 3H). MS (APCI) calculated for C₂₉H₃₇N₅O₃ m/z 503.3, found 504.3 (M+H)⁺.

(11) Tert-butyl 4-((2-butyl-4-((2,4-dimethoxybenzyl)amino)-1H-imidazo[4,5-c]quinolin-1-5 yl)methyl)benzylcarbamate. The synthetic protocol was the same as for (10), except 300 mg of (9) was used as the starting material and 2,4-dimethoxy benzylamine was used in place of benzylamine. Product was recrystallized from 3:1 hexane/ethyl acetate to obtain 272 mg (78% yield) of a spectroscopically pure product (>95% at 254 nm). ¹H NMR (400 MHZ, DMSO-d6) δ 9.64 (s, 1H), 8.16 (s, 1H), 7.91 (s, 1H), 7.60 (t, J=7.8 Hz, 1H), 7.34 (q, J=7.1, 6.1 Hz, 2H), 7.18 (d, J=8.0 Hz, 3H), 7.02 (d, J=8.0 Hz, 2H), 6.60 (d, J=2.3 Hz, 1H), 6.49 (dd, J=8.3, 2.4 Hz, 1H), 5.91 (s, 2H), 4.89 (s, 2H), 4.05 (d, J=6.2 Hz, 2H), 3.77 (s, 3H), 3.74 (s, 3H), 2.92 (t, J=7.7 Hz, 2H), 1.75-1.66 (m, 2H), 1.37-1.19 (m, 11H), 0.84 (t, J=7.3 Hz, 3H). MS (APCI) calculated for C₃₆H₄₃N₅O₄ m/z 609.3, found 610.3 (M+H)⁺.

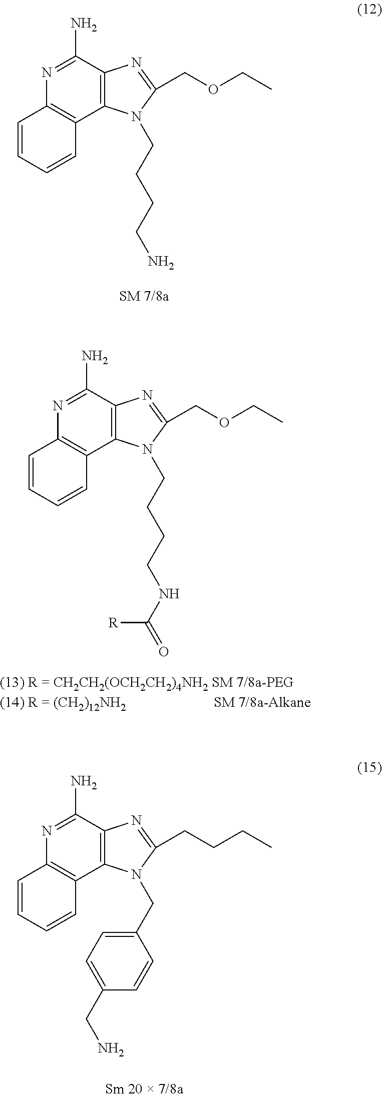

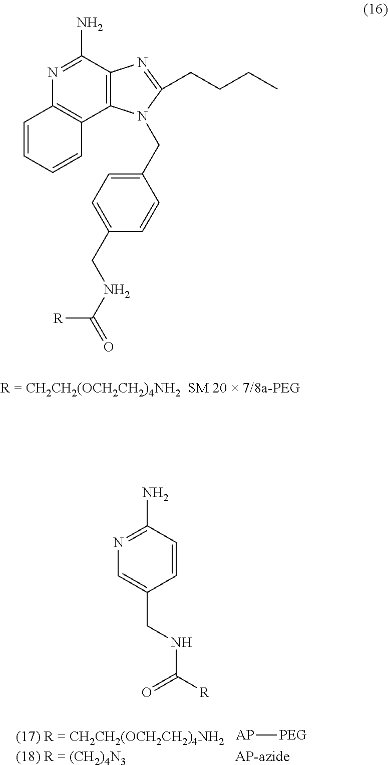

### Polymer Reactive Small Molecule Toll-Like Receptor-7/8 Agonists (TLR-7/8a) and Aromatic Heterocyclic Base Control Ligands Based on Aminopyridine (AP).

(12) SM 7/8a. 1-(4-aminobutyl)-2-(ethoxymethyl)-1H-imidazo[4,5-c]quinolin-4-amine. Simultaneous debenzylation and Boc removal was achieved by adding 36 mL of 98% H₂SO₄ (36.8 N) to 7.2 g (0.014 mol) of (10). The solution turned from faint yellow to cloudy orange over several minutes. Reaction progress was monitored by HPLC. After 3 hours, the reaction mixture was slowly added to 200 mL of DI H₂O and stirred at room temperature for 30 minutes. This mixture was filtered through celite and the resulting clear aqueous solution was adjusted to pH 10 using 10 M NaOH. The aqueous layer was extracted with 6×100 mL DCM. The organic layer was dried with Na₂SO₄ and then evaporated to dryness, yielding 4.03 g (89.6% yield) of a spectroscopically pure (>95% at 254 nm) white powder. ¹H NMR (400 MHZ, DMSO-d6) δ 8.02 (dd, J=16.6, 8.2 Hz, 1H), 7.63-7.56 (m, 1H), 7.47-7.38 (m, 1H), 7.30-7.21 (m, 1H), 6.55 (s, 2H), 4.76 (s, 2H), 4.54 (q. J=6.3, 4.4 Hz, 2H), 3.54 (q. J=7.0 Hz, 2H), 2.58 (t. J=6.9 Hz, 2H), 1.93-1.81 (m, 2H), 1.52 (m, 2H), 1.15 (t. J=7.0 Hz, 3H). MS (APCI) calculated for C₁₇H₂₃N₅O m/z 313.2, found 314.2 (M+H)⁺.

(13) SM 7/8a-PEG, 1-amino-N-(4-(4-amino-2-(ethoxymethyl)-1H-imidazo[4,5-c]quinolin-1-yl)butyl)-3,6,9,12-tetraoxapentadecan-15-amide. To 20 mL of ethyl acetate was added 500 mg (1.6 mmol, 1 eq) of (12), 281 mg (1.6 mmol, 1 eq) of CDMT and 643 mg (1.8 mmol, 1.1 eq) of Boc-15-amino-4,7,10,13-tetraoxapentadecanoic acid (PEG4), followed by the dropwise addition of 441 μL (4.0 mmol, 2.5 eq) of NMM, while stirring vigorously. After 16 hours at room temperature, the reaction mixture was filtered and then washed 3×50 mL with 1 M HCl. The organic phase was dried with Na₂SO₄ and then evaporated to dryness. The resulting solid purified by flash chromatography using a 2-15% methanol/dichloromethane gradient. The resulting clear oil was added to 5 mL of 30% TFA/DCM and reacted for 1 hour at room temperature. The TFA/DCM was removed by evaporation and the resulting residue was dissolved in 1M HCl and filtered. The filtrate was made alkaline by the addition of 10 M NaOH, followed by extraction with 3×50 mL of DCM. The organic phase was dried with Na₂SO₄ and evaporated to dryness to obtain 455 mg (51% yield) of spectroscopically pure (>95% at 254 nm) clear oil. ¹H NMR (400 MHz. DMSO-d6) δ 7.98 (d. J=8 Hz 1H), (7.83 (t. J=5.7 Hz, 1H), 7.60 (dd. J=8.4, 1.3 Hz, 1H), 7.43 (dd. J=8.4, 6.9 Hz, 1H), 7.25 (t. J=7.7 Hz, 1H), 6.56 (s, 2H), 4.75 (s, 2H), 4.59-4.50 (m, 2H), 4.07 (d. J=5.8 Hz, 4H), 3.59-3.39 (m, 16 H) 3.09 (q. J=6.5 Hz, 2H), 2.63 (t, J=5.9 Hz, 2H), 2.24 (t, J=6.5 Hz, 2H), 1.83 (m, 2H), 1.56 (t, J=7.5 Hz, 2H), 1.15 (t, J=7.0 Hz, 3H). MS (APCI) calculated for C₂₈H₄₄N₆O₆ m/z 560.3, found 561.3 (M+H)⁺.

(14) SM 7/8a-Alkane, 12-amino-N-(4-(4-amino-2-(ethoxymethyl)-1H-imidazo[4,5-c]quinolin-1-yl)butyl) dodecanamide. To 20 mL of ethyl acetate was added 200 mg (0.64 mmol, 1 eq) of (12), 112 mg (0.64 mmol, 1 eq) of CDMT and 222 mg (0.70 mmol, 1.1 eq), of N-boc-aminodecanoic acid followed by the dropwise addition of 176 μl (1.6 mmol, 2.5 eq) of NMM while stirring vigorously. After 16 hours at room temperature, the reaction mixture was filtered and washed 3×50 mL with 1 M HCl. The organic phase was dried with Na₂SO₄ and then evaporated to dryness. The resulting solid was suspended in 5 mL of 30% TFA/DCM and reacted for 1 hour at room temperature. The TFA/DCM was removed by evaporation and the resulting residue was dissolved in 1M HCl and filtered. The filtrate was made alkaline by the addition of 10 M NaOH, followed by extraction with 3×50 mL of DCM. The organic phase was then dried with Na₂SO₄ and evaporated to dryness to obtain 279 mg (85.4% yield) of spectroscopically pure (>95% at 254 nm) white solid. ¹H NMR (400 MHZ, DMSO-d6) δ 7.98 (d, J=8.1 Hz, 1H), 7.74 (t, J=5.7 Hz, 1H), 7.60 (d, 8 Hz, 1H), 7.42 (t, J=7.6 Hz, 1H), 7.24 (t, J=7.5 Hz, 1H), 6.56 (s, 2H), 4.75 (s, 2H), 4.53 (t, J=7.9 Hz, 2H), 3.54 (q, J=7.0 Hz, 2H), 3.07 (q, J=6.4 Hz, 2H), 2.60 (t, J=7.1 Hz, 2H), 1.97 (t, J=7.4 Hz, 2H), 1.87-1.78 (m, 2H), 1.55 (t, J=7.6 Hz, 2H), 1.43-1.34 (m, 5H), 1.24-1.10 (m, 18H). MS (APCI) calculated for C₂₉H₄₆N₆O₂ m/z 510.4, found 511.4 (M+H)⁺.

(15) SM 20×7/8a, 1-(4-(aminomethyl)benzyl)-2-butyl-1H-imidazo[4,5-c]quinolin-4-amine. Deprotection of (11) required milder conditions as compared with (12) so as to avoid removal of the xylene diamine linker. Simultaneous removal of the 2,4-dimethoxybenzyl and Boc groups was achieved by adding 300 mg of (11) to a 30 mL solution of 40% TFA/DCM that was stirred at room temperature for 30 hours. The reaction mixture turned from clear to deep red over several hours and the reaction was monitored by HPLC. After completion, the reaction mixture was evaporated to dryness and the resulting red oil was suspended in 200 mL of 1 M HCl. Insoluble pink material was removed by filtration and the resulting clear aqueous solution was adjusted to pH 10 using 10 M NaOH. The aqueous layer was extracted 6×100 mL using DCM as the organic phase. The organic layer was dried with Na₂SO₄ and evaporated to dryness, yielding 172 mg (89.6% yield) of a spectroscopically pure (>95% at 254 nm) white powder. ¹H NMR (400 MHZ, DMSO-d6) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅ m/z 359.2, found 360.3 (M+H)⁺.

(16) SM 20×7/8a-PEG, 1-(4-(aminomethyl)benzyl)-2-butyl-1H-imidazo[4,5-c]quinolin-4-amine. The same reaction conditions and purification scheme were used as for the preparation of (13), except 100 mg of (15) was used in place of (12). 126.2 mg (96% yield) of spectroscopically pure (>95% at 254 nm) clear oil was obtained. ¹H NMR (400 MHZ, DMSO-d6) δ 8.31 (t, J=6.0 Hz, 1H), 7.93 (d, J=8.4 Hz, 1H), 7.78 (d, J=8.3 Hz, 1H), 7.71 (s, 4H), 7.61 (t, J=7.8 Hz, 1H), 7.35 (t, J=7.8 Hz, 1H), 7.18 (d, J=8.0 Hz, 2H), 7.00 (d, J=8.0 Hz, 2H), 5.92 (s, 2H), 4.20 (d, J=5.9 Hz, 2H), 3.62-3.44 (m, 16H), 3.00-2.90 (m, 4H), 2.33 (t, J=6.4 Hz, 2H), 1.75-1.67 (m, 2H), 1.37 (q, J=7.4 Hz, 2H), 0.85 (t, J=7.3 Hz, 3H). MS (APCI) calculated for C₃₃H₄₆N₆O₅ m/z 606.4, found 607.3 (M+H)⁺.

(17) AP-PEG, 1-amino-N-((6-aminopyridin-3-yl)methyl)-3,6,9,12-tetraoxapentadecan-15-amide. The same reaction conditions and purification scheme were used as for the preparation of (13), except 50 mg of tert-Butyl 5-(aminomethyl)-2-pyridinylcarbamate was used in place of (12). 73 mg (88% yield) of spectroscopically pure (>95% at 254 nm) clear oil was obtained. 1H NMR (400 MHZ, DMSO-d6) δ 8.32 (t, J=5.9 Hz, 1H), 7.76 (d, J=2.1 Hz, 1H), 7.66 (dd, J=9.0, 2.2 Hz, 1H), 7.51 (s, 2H), 6.81 (d, J=9.0 Hz, 1H), 4.10 (d, J=5.8 Hz, 2H), 3.67-3.37 (m, 16H), 2.96 (s, 2H), 2.53 (p, J=1.9 Hz, 1H), 2.43 (p, J=1.9 Hz, 1H), 2.33 (t, J=6.4 Hz, 2H). MS (APCI) calculated for C₁₇H₃₀N₄O₅ m/z 370.2, found 371.2 (M+H)⁺.

(18) AP-azide, N-((6-aminopyridin-3-yl)methyl)-5-azidopentanamide. The same reaction conditions and purification scheme were used as for the preparation of (13), except 50 mg of tert-Butyl 5-(aminomethyl)-2-pyridinylcarbamate was used in place of (12). 21.4 mg (39% yield) of spectroscopically pure (>95% at 254 nm) clear oil was obtained. ¹H NMR (400 MHZ, DMSO-d6) δ 8.25 (t, J=5.7 Hz, 1H), 7.75 (d, J=2.2 Hz, 1H), 7.65-7.57 (m, 1H), 7.22 (s, 2H), 6.75 (d, J=8.9 Hz, 1H), 4.07 (d, J=5.8 Hz, 2H), 2.43 (m, 2H), 2.11 (t, J=7.0 Hz, 2H), 1.50 (m, 4H). MS (APCI) calculated for C₁₁H₁₆N₆O m/z 248.1, found 249.1 (M+H)⁺.

### Synthesis of SM TLR-7/8a Dye Conjugates

(19) SM 7/8a-AF488.

The AF488 dye conjugate of the small molecule TLR-7/8a was synthesized by reacting 2 mg (2.3 μmoles, 1 eq) of Alexa Fluor® 488 carboxylic acid tetrafluorophenyl ester with 0.85 mg (2.7 μmoles, 1.2 eq) of (12) in 300 μL of anhydrous DMSO. The reaction was monitored by HPLC and the product, (19), was purified by semi-prep HPLC using a 25% to 35% ACN/H₂O gradient over 16 minutes. The reaction mixture was injected over 3 runs. Fractions containing (19) were consolidated, frozen and lyophilized to yield 1.6 mg (85.5% yield) of spectroscopically pure (>95% at 254 nm) product. MS (ESI) calculated for C₃₈H₃₃N₇O₁₁S₂ m/z 827.2, found 827.7 (M+H)⁺.

(20) SM 7/8a-IRDye800

For the IR Dye conjugate of the SM 7/8a, a PEG spacer was required to increase solubility. The same reaction conditions and purification scheme were used as for the preparation of (19), except 4 mg (3.4 μmoles, 1 eq) of IR Dye 800cw NHS ester was used as the dye and reacted with 2.3 mg (4.1 μmoles, 1 eq) of (13). 3.8 mg (71% yield) of spectroscopically pure (>95% at 254 nm) product was obtained. MS (ESI) calculated for C₇₄H₉₆N8020S₄ m/z 1546, found 1547 (M+H)⁺.

### Synthesis of Amine-Reactive HPMA-Based Copolymers

The N-(2-hydroxypropyl) methacrylamide (HPMA)-based statistical copolymer, p[(HPMA)-co-(Ma-ε-Ahx-TT), was synthesized by free radical solution polymerization as previously described⁸. Briefly, a mixture of HPMA (9.8 wt %), 2-Methyl-N-[6-oxo-6-(2-thioxo-thiazolidin-3-yl)-hexyl]-acrylamide (Ma-ε-Ahx-TT) (5.2 wt %) and azobisisobutyronitrile (AIBN) (1.5 wt %) were dissolved in DMSO (83.5 wt %) and polymerized at 60° C. for 6 hours under argon atmosphere. The polymer was precipitated from a 1:1 mixture of acetone and diethyl ether and then dissolved into methanol and precipitated from a 3:1 mixture of acetone and diethyl ether. The content of TT reactive groups determined by UV-Vis spectrophotometry was 14.8 mol % (ε₃₀₅=10,300 L/mol); the weight- and number-average molecular weights determined by size exclusion chromatography (SEC) were M_{w}=31,850 g/mol and Mₙ=20,330 g/mol, respectively.

### Synthesis of Amine-Reactive NIPAM-Based (Thermo-Responsive) Copolymers

The N-isopropylacrylamide (NIPAM)-based statistical copolymer p [(NIPAM)-co-(Ma-Ahx-TT)] was prepared by free radical solution polymerization as described elsewhere⁹. Briefly, a mixture of NIPAM (10.2 wt %), Ma-8-Ahx-TT (4.8 wt %) and AIBN (1.5 wt %) was dissolved in DMSO (83.5 wt %) and polymerized at 60° C. for 18 hours under argon atmosphere. The reaction mixture was diluted with an HCl aqueous solution (pH 2) and then extracted with dichloromethane (3×). The combined organic phases were dried and evaporated. The resulting residue was dissolved in methanol and precipitated into a 3:1 mixture of acetone and diethyl ether. The content of TT reactive groups determined by UV-Vis spectrophotometry was 10.2 mol % (ε_{305=10,300} L/mol); the weight- and number-average molecular weights determined by SEC were M_{w}=26,830 g/mol and Mₙ=17,650 g/mol, respectively.

### Synthesis of Polymer-TLR-7/8a (Poly-7/8a) Conjugates

Example: To generate p[(HPMA)-co-(Ma-ε-Ahx-PEG4-7/8a)] with an agonist density of ˜10 mol % TLR-7/8a, 10 mg (8.4 μmole TT, 1 eq) of p[(HPMA)-co-(Ma-ε-Ahx-TT)] with ˜14 mol % TT was added to 1 mL of anhydrous methanol. To this solution, 470 μL (4.7 mg, 6.0 μmole, 0.7 eq) of a 10 mg/ml solution of (13) (SM 7/8-PEG) in anhydrous DMSO was slowly added while stirring vigorously. After 16 hours, 1.25 mg (16.8 μmole, 2 eq) of 1-amino-2-propanol was added to remove unreacted TT groups. After an additional 2 hours, the reaction mixture was dialyzed against methanol using Spectra/Por7 Standard Regenerated Cellulose dialysis tubing with a molecular weight cut-off (MWCO) of 25 kDa (Spectrum Labs, Rancho Dominguez, CA). The dialysis tube was suspended in 1000 mL of methanol and the dialysis buffer was changed twice each day for 3 days. The methanol solution containing Poly-7/8a was evaporated to dryness and yielded 11.4 mg of p[(HPMA)-co-(Ma-ε-Ahx-PEG4-7/8a)]. The content of 7/8a-PEG determined by UV-Vis spectrophotometry was 7.9 mol % 7/8a (ε_{325=5,012} L/mol); the weight- and number-average molecular weights determined by SEC were M_{w}=55,680 g/mol and Mₙ=33,850 g/mol, respectively.

Synthesis of Second-Generation TRPP-7/8a with ESE Coil Peptide
TRPP: p[(HPMA)-Co-(PgMA)]-Block-p (DEGMA) Second generation TRPP-7/8a were produced as thermo-responsive A-B type di-block copolymers by RAFT polymerization in two synthetic steps. The hydrophilic block A was prepared by copolymerizing HPMA with N-propargyl methacrylamide (PgMA) using 4,4′-azobis(4-cyanovaleric acid) (ACVA) as an initiator and 4-Cyano-4-(phenylcarbonothioylthio) pentanoic acid (CTP) as a chain transfer agent in molar ratios [M]: [CTP]: [ACVA]=142:2:1 in 1,4-dioxane/H₂O mixture. Briefly, a mixture of 7.6 mg CTP (27.3 μmol) and 3.8 mg ACVA (13.7 μmol) was dissolved in 647 μL of 1,4-dioxane and added to the solution of 250.0 mg HPMA (1.75 mmol) and 23.9 mg PgMA (0.19 mmol) in 1293 μL of DI H₂O. The reaction mixture was thoroughly bubbled with Argon and polymerized in sealed glass ampoules at 70° C. for 6 h. The resulting copolymer was isolated by precipitation into a 3:1 mixture of acetone and diethyl ether and purified by gel filtration using a Sephadex™ LH-20 cartridge with methanol as the eluent. The polymer solution was concentrated in vacuo and precipitated to diethyl ether yielding 131.5 mg of the p[(HPMA)-co-(PgMA)] polymer. The content of dithiobenzoate (DTB) end groups determined by UV-Vis spectrophotometry was n_{DTB}=0.106 mmol/g (ε₃₀₂=12,100 L/mol) corresponding to the functionality of the polymer chain f=0.98. The weight- and number-average molecular weights determined by SEC were M_{w}=9,809 g/mol and Mₙ=9,229 g/mol, respectively. The content of PgMA determined by ¹H NMR was 9.8 mol %.

The hydrophilic polymer block A bearing DTB terminal groups was subjected to a chain-extension polymerization through the RAFT mechanism with di(ethylene glycol) methyl ether methacrylate (DEGMA) to introduce the thermo-responsive polymer block B. Briefly, a mixture of 50.0 mg p[(HPMA)-co-(PgMA)] (5.31 μmol˜DTB gr.), 53.0 mg DEGMA (0.28 mmol) and 0.30 mg ACVA (1.06 μmol) was dissolved in 477 μL of 1,4-dioxane/H₂O (2:1) solution and thoroughly bubbled with argon gas before sealing the glass ampoule reaction vessel and carrying out the reaction at 70° C. for 18 h. The di-block polymer was isolated by precipitation to diethyl ether followed by re-precipitation from methanol to 3:1 mixture of acetone and diethyl ether to yield 84.4 mg of the product. The content of dithiobenzoate (DTB) end groups determined by UV-Vis spectrophotometry was n_{DTB}=31.1 μmol/g (8302=12/,100 L/mol).

To remove the DTB end groups, the polymer and 12.9 mg of AIBN (0.79 μmol) were dissolved in 844 μL of DMF and the solution was heated to 80° C. for 2 h. The resulting polymer was isolated by precipitation in diethyl ether and purified by gel filtration using a Sephadex™ LH-20 cartridge with methanol as the eluent. The polymer solution was concentrated in vacuo and precipitated in diethyl ether yielding 72.4 mg of the product. The weight- and number-average molecular weights determined by SEC were M_{w}=22,020 g/mol and Mₙ=16,790 g/mol, respectively. The transition temperature (TT) of the polymer, determined by DLS, was 38° C. at 1.0 mg/mL 15 M PBS (pH 7.4).

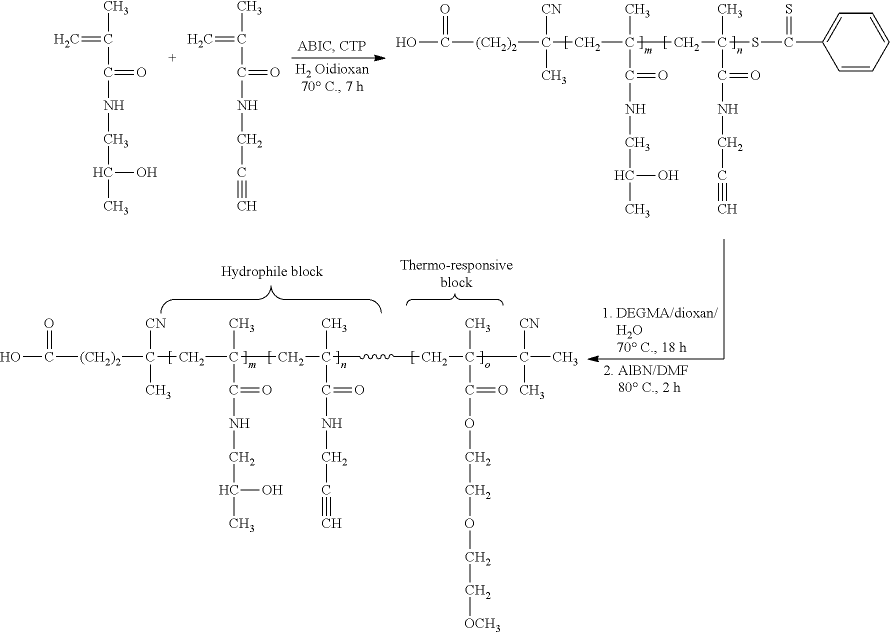

### Attachment of TLR-7/8a, ESE Coil Peptide and Fluorophore to TRPP

Different ligands (TLR-7/8a, ESE-coil peptide, scrambled peptide or fluorophore) functionalized with an azide group were attached to TRPP through the propargyl side chain moieties distributed along the hydrophilic block A of the copolymer by copper catalyzed 1,3 dipolar cycloaddition reaction. Reaction progress was monitored by HPLC.

Example: A mixture of 20.0 mg TRPP (7.1 μmol propargyl group), 1.0 mg TLR-7/8a-azide (2.1 μmol), 0.4 mg Carboxyrhodamine 110-azide (0.7 μmol), 4.6 mg ESE-coil peptide-azide (1.4 μmol) and 1.1 mg TBTA (2.1 μmol) was dissolved in 460 μL of DMSO and the solution was thoroughly bubbled with Argon. Then, 0.84 mg sodium ascorbate (4.2 μmol) in 168 μL of degassed water was added. Finally, a solution of 0.54 mg CuSO₄ in 108 μL of degassed water was pipetted to the reaction mixture to initiate the “click” reaction. The reaction was performed overnight at 45° C. until no unreacted ligands were detected by HPLC. The reaction mixture was diluted (1:1) with a saturated solution of EDTA in 0.15 M PBS (pH 7.4) and the conjugate was purified by gel filtration using a Sephadex™ PD-10 column with H₂O as the eluent. The resulting conjugate was isolated from an aqueous solution by lyophilisation yielding 18.6 mg of the product. See FIG. 21.

### Attachment of HIV Gag-KSK to Fluorescently Labelled TRPP-ESE Conjugate Via the Coiled Coil Interaction

Formation of TRPP-(-coil-coil)-Gag complex was performed in PBS buffer by mixing TRPP-ESE with HIV Gag-KSK at 1.5/1.0 molar ratio (based on coil peptides). Formation of the coiled-coil complex was measured using SEC on MicroSuperose 12 column and by analytical ultracentrifugation (AUC) 1 hour after mixing. See FIG. 22.

### Determination of TLR-7/8a and Fluorophore Content on Polymers

The amount of ligand attached to the copolymers was determined by UV-Vis spectroscopy using the Beer-Lambert law relationship (A=ε*c; where A=absorption and c=mol/L). Samples were suspended in solutions of 1% triethylamine/methanol at known densities (mg/mL) and added to quartz cuvettes with a path length of 1 cm. Absorption was recorded over a spectrum from 250-775 nm using a Lambda25 UV-Vis spectrometer from Perkin Elmer. For example, a 0.1 mg/mL solution of Poly-7/8a in 1% triethylamine/methanol (λₘₐₓ=325 nm, ε₃₂₅=5012 L/mol) has an optical density (OD, arbitrary units) of 0.25 at 325 nm. The concentration of TLR-7/8 can be calculated by solving for c in the Beer-Lambert law relationship and is 5e-5 mol/L, which can be expressed as the amount of TLR-7/8a per mass of copolymer (5e-4 mmol/mg).

The Beer-Lambert relationship was used to determine the amount of ligand molecules and dyes attached to the polymers based on known extinction coefficients.

| | | Extinction | |
|---|---|---|---|
| | | coefficient, | |
| | Max | (L/mol) 1% | |
| | absorption | triethylamine/ | |
| Ligand | ( [symbol] max, nm) | methanol | A₃₂₅/Aₘₐₓ |
| Aminopyridine | 305 | 3,511 | — |
| TLR-7/8a | 325 | 5,012 | 1.00 |
| (SM 7/8a) | | | |
| AF488 | 495 | 167,415 | 0.12 |
| Cruz Fluor 8 | 775 | 71,493 | 0.09 |

Methods table 1: Absorption maxima and extinction coefficients were determined for different ligand and dye molecules in 1% triethylamine/methanol. Note that for copolymers with both TLR-7/8a and dye (AF488 or Cruz Fluor 8), the contribution of absorption at 325 nm by the dye was determined using the relationship described by A₃₂₅/Aₘₐₓ.

### Agonist Density (Mol % 7/8a) Determination

UV-Vis can be used to estimate the agonist density (mol %) of co-monomers. Mol % of co-monomer y, for a statistical copolymer comprised of monomers x and y is estimated using the following relationship:

$$mol {\%}_{y} = ( \frac{1}{1 + ( \frac{ρ \times ε}{A \times {Mw}_{x}} - \frac{{Mw}_{y}}{Mwx} )} ) * 100$$

mol % _{y} (agonist density)=percentage of copolymer that is y (e.g., TLR-7/8a containing monomer), for copolymer comprised of x and y monomers
ρ=volumetric mass density (mg/mL) of copolymer during UV-Vis measurement
ε=molar extinction coefficient for monomer y (e.g. for TLR-7/8a=5,012)
A=Absorbance
Mwₓ=molecular weight (g/mol) of majority monomer
Mw_{y}=molecular weight (g/mol) of minority monomer

### Example Calculation

For poly-7/8a comprised of the majority monomer HPMA (Mw_{HPMA}=143.2) and minority monomer containing the TLR-7/8a (MA-Ahx-PEG4-7/8a; Mw_{MA-PEG4}-7/8a=741.9) that is suspended in methanol at 0.1 mg/mL and measures an average absorbance of 0.25 at 325 nm, the mol % of the minority unit, MA-PEG4-7/8a is:

$$mol {\%}_{MA - Ahx - PEG 4 - 7 / 8 a} = ( \frac{1}{1 + ( \frac{0.1 \times 5012}{0.25 \times 143.2} - \frac{741.9}{143.2} )} ) * 100 = 10.2 \%$$

### Synthesis of Conjugatable TLR-4 Agonists

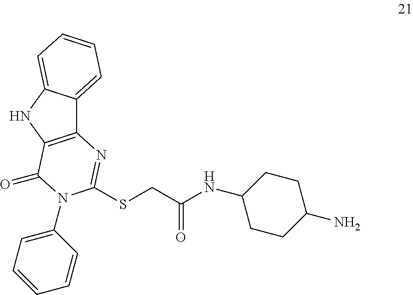 [SMILES: NC1CCC(NC(=O)CSc2nc3c([nH]c4ccccc43)c(=O)n2-c2ccccc2)CC1]

(21) PI-NH₂. tert-butyl (4-(2-((4-oxo-3-phenyl-4,5-dihydro-3H-pyrimido[5,4-b]indol-2-yl)thio) acetamido)cyclohexyl) carbamate. The pyrimidoindole carboxylic acid precursor (2-((4-oxo-3-phenyl-4,5-dihydro-3H-pyrimido[5,4-b]indol-2-yl)thio) acetic acid) was prepared as recently described¹⁰ mg of this compound (0.28 mmol, 1 eq) and 67.1 mg (0.31 mmol, 1.1 eq) of N-Boc-trans-1,4-cyclohexanediamine were then added to 2 mL of DMF with triethylamine 80 μL Et₃N (0.56 mmol, 2 eq). A solution of 118 mg (0.31 mmol, 1.1 eq) of HATU in 400 μL of DMF was then added to the reaction mixture. The reaction was stirred at RT for 24 h. The solution was concentrated and recrystallized from methanol to provide the Boc-protected product as a white solid (108 mg, 70% yield). ¹H NMR (500 MHZ, DMSO-d6) d□12.1 (s, 1H), 8.08 (d, J=8, 1H), 7.63-7.61 (br m, 2H), 7.53 (t, J=8, 2H), 7.50-7.48 (br m, 4H), 7.30 (t, J=6, 1H), 6.72 (d, J=8, 1H), 3.89 (s, 2H), 3.43 (br s, 1H), 3.17 (br s, 1H), 1.76 (br t, J=13, 4H), 1.38 (s, 9H), 1.30-1.14 (br m, 4H). ¹³C NMR (500 MHZ, DMSO-d6) d□166.4, 155.4, 153.0, 139.4, 137.7, 136.6, 130.0, 129.7, 129.4, 128.5, 127.8, 120.8, 120.6, 119.7, 114.7, 113.3, 77.9, 48.1, 46.2, 37.2, 31.7, 31.6, 28.8. TLC: 100% Ethyl acetate, Rf 0.7. HRMS: m/z calcd for C₂₉H₃₃N₅O₄S [M+Na]⁺ 570.2, observed 570.2. 50 mg of the resulting Boc protected compound was then added to 5 mL of 30% TFA/DCM and reacted for 1 hour at room temperature. The TFA/DCM was removed by evaporation and the resulting residue was dissolved in 1M HCl and filtered. The filtrate was made alkaline by the addition of 10 M NaOH, followed by extraction with 3×50 mL of DCM. The organic phase was dried with Na₂SO₄ and evaporated to dryness to obtain 33 mg (80.8% yield) of a spectroscopically pure (>95% at 254 nm) white solid. MS (ESI) calculated for C₂₄H₂₅N₅O₂S m/z 447.17, found 448.2 (M+H)⁺.

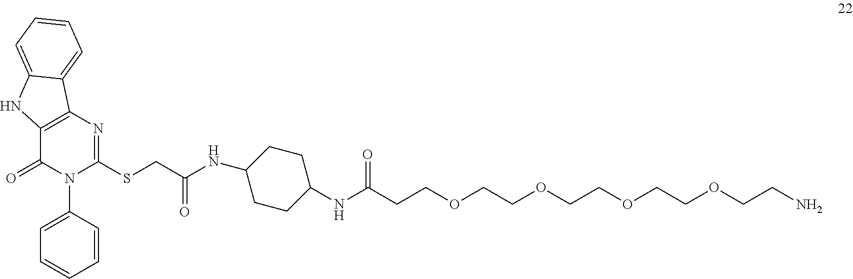 [SMILES: NCCOCCOCCOCCOCCC(=O)NC1CCC(NC(=O)CSc2nc3c([nH]c4ccccc43)c(=O)n2-c2ccccc2)CC1]

(22) PI-PEG, 1-amino-N-(4-(2-((4-oxo-3-phenyl-4,5-dihydro-3H-pyrimido[5,4-b]indol-2-yl)thio) acetamido)cyclohexyl)-3,6,9,12-tetraoxapentadecan-15-amide. To a 1:2 solution of 5 mL of methanol/DCM was added 15.0 mg (0.03 mmol, 1 eq) of (21), 5.9 mg (0.03 mmol, 1 eq) of CDMT and 18.4 mg (0.05 mmol, 1.5 eq) of Boc-15-amino-4,7,10,13-tetraoxapentadecanoic acid (PEG4), followed by the dropwise addition of 9.25 μL (0.08 mmol, 2.5 eq) of NMM, while stirring vigorously. After 16 hours at room temperature, the reaction mixture was filtered and then washed 3×50 mL with 1 M HCl. The organic phase was dried with Na₂SO₄ and then evaporated to dryness to yield solid that was purified by semi-prep HPLC using a 33-55% ACN/H₂O gradient over 14 minutes. 11 mg (41% yield) of white solid was obtained and then added to 1 mL of 30% TFA/DCM and reacted for 1 hour at room temperature. The TFA/DCM was removed by evaporation and the resulting residue was dissolved in 1M HCl and filtered. The filtrate was made alkaline by the addition of 10 M NaOH, followed by extraction with 3×50 mL of DCM. The organic phase was dried with Na₂SO₄ and evaporated to dryness to obtain 7 mg (73% yield) of spectroscopically pure (>95% at 254 nm) white solid. ¹H NMR (400 MHZ, DMSO-d6) δ 8.19 (d, J=7.8 Hz, 1H), 8.06 (d, J=8.0 Hz, 1H), 7.70 (t, J=7.9 Hz, 1H), 7.60 (qd, J=5.2, 1.9 Hz, 2H), 7.55-7.41 (m, 3H), 7.30-7.20 (m, 1H), 4.11 (s, 2H), 3.87 (s, 2H), 3.56 (t, J=6.4 Hz, 2H), 3.56-3.40 (m, 12H), 3.17 (s, 3H), 2.63 (t, J=5.8 Hz, 1H), 2.25 (t, J=6.5 Hz, 2H), 1.81-1.68 (m, 4H), 1.34-1.08 (m, 8H), 0.92-0.78 (m, 1H). MS (APCI) calculated for C₃₅H₄₆N₆O₇S m/z 694.3, found 695.3 (M+H)⁺.

### Synthesis of Polymer-TLR4a Conjugates (PP-PI)

Example: The polymer-particle forming TLR-4a conjugate (PP-PI) described in this study was prepared by reacting (22) with amine reactive p[(HPMA)-co-(Ma-β-Ala-TT)]. In short, 5 mg (3.7 μmol, TT, 1 eq) of p[(HPMA)-co-(Ma-β-Ala-TT)] with ˜11.7 mol % TT was added to 500 μL of anhydrous methanol. To this solution was added 2.6 mg (3.7 μmol, 1 eq) of a 10 mg/ml solution of (22) in anhydrous DMSO while stirring vigorously. After 16 hours, 2 eq of 1-amino-2-propanol was added to remove unreacted TT groups. After an additional 2 hours, the reaction mixture was dialyzed against methanol using Spectra/Por7 Standard Regenerated Cellulose dialysis tubing with a molecular weight cut-off (MWCO) of 25 kDa (Spectrum Labs, Rancho Dominguez, CA). The dialysis tube was suspended in 1000 mL of methanol and the dialysis buffer was changed twice each day for 3 days. The methanol solution containing Poly-PEG-PI was evaporated to dryness and yielded 6.7 mg of p[(HPMA)-co-(Ma-β-Ala-PEG-PI)]. The content of PI-PEG determined by UV-Vis spectrophotometry was 6.3 mol % (ε₃₄₀=7,272 L/mol). 2,744±384.8 nm z-average diameter at 0.1 mg/mL PBS. 414.1±135.4 nm z-average diameter at 0.1 mg/mL PBS.

### Synthesis of Conjugatable Pam2cys (TLR-2/6a)

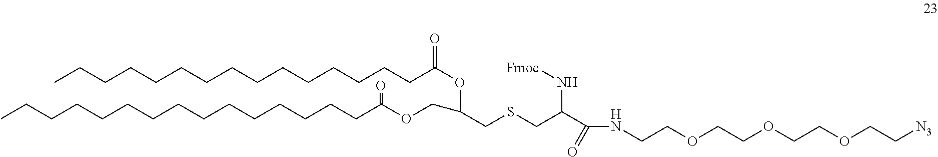

(23) Pam2Cys-PEG-N3 14-((((9H-fluoren-9-yl) methoxy) carbonyl)amino)-1-azido-13-oxo-3,6,9-trioxa-16-thia-12-azanonadecane-18,19-diyl dipalmitate, 1-amino-N-(4-(2-((4-oxo-3-phenyl-4,5-dihydro-3H-pyrimido[5,4-b]indol-2-yl)thio) acetamido)cyclohexyl)-3,6,9,12-tetraoxapentadecan-15-amide. To a 20 mL solution of 1:1 DCM/Methanol, was added 100 mg (0.11 mmol, 1 eq) of Fmoc-protected Pam2Cys-COOH (Fmoc-Cys ((RS)-2,3-di(palmitoyloxy)-propyl)-OH) (Bachem, Bubendorf, Switzerland) 27 mg (0.12 mmol, 1.1 eq) of Amino-11-azido-3,6,9-trioxaundecane and 20 mg (0.11 mmol, 1 eq) of CDMT, followed by the dropwise addition of 25 μl (0.22 mmol, 2.0 eq) of NMM, while stirring vigorously. After 16 hours at room temperature, the reaction mixture was filtered and then washed 3×50 mL with 1 M HCl. The organic phase was dried with Na₂SO₄ and then evaporated to dryness to yield a white solid that was further purified by flash column chromatography using 0-10% methanol/DCM gradient. Fractions were combined and evaporated to dryness to obtain 75.6 mg (62% yield) of spectroscopically pure (>95% at 254 nm by TLC) white solid. MS (APCI) calculated for C₆₁H₉₉N₅O₁₀S m/z 1093.7, found 1113 (M+H₃O)⁺ and 1208 (M+TFA)⁺.

### Synthesis of Polymer-2/6 Conjugates (PP-Pam2Cys)

Example: The polymer-particle forming TLR-2/6a conjugate described in this study was prepared by reacting (23) with amine reactive p[(HPMA)-co-(Ma-β-Ala-TT)] in a 3 step reaction. In the first step, 5 mg (3.7 μmol TT, 1 eq) of p[(HPMA)-co-(Ma-β-Ala-TT)] with ˜11.7 mol % TT was added to 500 μL of anhydrous methanol. To this solution was added 98 μL (1.96 mg, 3.7 μmol, 1 eq) of a 10 mg/ml solution of the cross-linker, DBCO-PEGANH2, in anhydrous DMSO while stirring vigorously. After 2 hours, 204 μL (2.04 mg, 3.7 μmol, 1 eq) of a 10 mg/mL solution of (23) was then added while stirring the reaction mixture vigorously. After 16 hours, 2 eq of 1-amino-2-propanol was added to remove unreacted TT groups. After an additional 2 hours, the reaction mixture was dialyzed against methanol using Spectra/Por7 Standard Regenerated Cellulose dialysis tubing with a molecular weight cut-off (MWCO) of 25 kDa (Spectrum Labs, Rancho Dominguez, CA). The dialysis tube was suspended in 1000 mL of a 1:1 methanol/DCM solution and the dialysis buffer was changed twice over 1 day. The methanol solution containing Poly-PEG-Pam2Cys (Fmoc) was evaporated to dryness and then suspended in a 1 mL solution of 20% Piperidine/DMF for 1 hour to remove the Fmoc group. The reaction mixture was then dialyzed again against a solution of 1:1 methanol/DCM and the dialysis buffer was changed after 15 minutes, and then twice per day for 3 days. The methanol solution containing Poly-PEG-Pam2Cys was evaporated to dryness and yielded 8.1 mg of p[(HPMA)-co-(Ma-β-Ala-PEG-Pam2Cys)]. The content of Pam2Cys-PEG determined by UV-Vis spectrophotometry was 4.5 mol % Pam2Cys as determined using the TNBSA assay to measure primary amine content (8420=11,500 L/mol). 2,744±384.8 nm z-average diameter at 0.1 mg/mL PBS.

Formulation of MPL (TLR-4a) and CpG (TLR-9a) with particulate carriers Both Monophosphoryl Lipid A (MPL) and CpG ODN 1826 were purchased from Invivogen as vaccine grade adjuvants. Alum/MPL for immunizations was comprised of a solution of PBS with 0.1 mg/mL MPL and 1 mg/mL Aluminum Hydroxide (Alhydrogel, Invivogen) that was allowed to incubate at room temperature for 2 hours prior to immunization. Polymer/CpG poly(plex) particles were prepared by formulating 16 kD Poly(L-lysine hydrochloride) (Alamanda Polymers, Huntsville, AL, USA) linear polymers with CpG ODN 1826 at 20:1 N:P in PBS.

### REFERENCES

1. Cinelli, M. A., Cordero, B., Dexheimer, T. S., Pommier, Y. & Cushman, M. Synthesis and biological evaluation of 14-(aminoalkyl-aminomethyl) aromathecins as topoisomerase I inhibitors: Investigating the hypothesis of shared structure-activity relationships. Bioorgan Med Chem 17, 7145-7155 (2009).
2. Kaminski, Z. J. 2-Chloro-4,6-Dimethoxy-1,3,5-Triazine-a New Coupling Reagent for Peptide-Synthesis. Synthesis-Stuttgart, 917-920 (1987).
3. Nanba, R. J., Iizuka, Takao (JP), Ishii, Takeo (JP) (TERUMO CORP (JP), 1999).
4. Russo, C. et al. Small molecule Toll-like receptor 7 agonists localize to the MHC class II loading compartment of human plasmacytoid dendritic cells. Blood 117, 5683-5691 (2011).
5. Shukla, N. M., Malladi, S. S., Mutz, C. A., Balakrishna, R. & David, S. A. Structure-activity relationships in human toll-like receptor 7-active imidazoquinoline analogues. J Med Chem 53, 4450-4465 (2010).
6. Shukla, N. M. et al. Syntheses of fluorescent imidazoquinoline conjugates as probes of Toll-like receptor 7. Bioorg Med Chem Lett 20, 6384-6386 (2010).
7. Gerster, J. F. et al. Synthesis and structure-activity-relationships of 1H-imidazo[4,5-c] quinolines that induce interferon production. J Med Chem 48, 3481-3491 (2005).
8. Subr, V. & Ulbrich, K. Synthesis and properties of new N-(2-hydroxypropyl)-methacrylamide copolymers containing thiazolidine-2-thione reactive groups. React Funct Polym 66, 1525-1538 (2006).
9. Hruby, M. et al. New bioerodable thermoresponsive polymers for possible radiotherapeutic applications. Journal of controlled release: official journal of the Controlled Release Society 119, 25-33 (2007).
10. Chan, M. et al. Identification of substituted pyrimido[5,4-b]indoles as selective Toll-like receptor 4 ligands. J Med Chem 56, 4206-4223 (2013).

The invention claimed is:

1. An adjuvant comprising Pattern Recognition Receptor (PRR) agonist molecules linked to linear or branched unimolecular polymer chains that are capable of undergoing particle formation in aqueous conditions, or in aqueous conditions in response to external stimuli.

2. The adjuvant according to claim 1, wherein the polymer is a thermo-responsive polymer.

3. The adjuvant according to claim 1 or claim 2, wherein the adjuvant is capable of assembling into particles in response to a temperature shift.

4. The adjuvant according to any receding claim, wherein the particles are a size capable of being phagocytosed, or larger.

5. The adjuvant according to any preceding claim, wherein the adjuvant is capable of assembling into particles of sizes between about 20 nm and about 10,000 nm.

6. The adjuvant according to any preceding claim, wherein the adjuvant is for local administration to a specific tissue, site, or region of the body.

7. The adjuvant according to any preceding claim, wherein the adjuvant is capable of being substantially retained in the body at the site of administration.

8. The adjuvant according to any of claims 2 to 7, wherein the thermo-responsive polymer has a lower critical solution temperature (LCST) of ≤40° C.

9. The adjuvant according to any of claims 2 to 8, wherein the thermo-responsive polymer is responsive to a temperature shift from below body temperature to body temperature.

10. The adjuvant according to any of claims 2 to 9, wherein the thermo-responsive polymer has a lower critical solution temperature of between 24° C. and 36° C.

11. The adjuvant according to any of claims 2 to 7, wherein the thermo-responsive polymer has a lower critical solution temperature (LCST) higher than normal body temperature.

12. The adjuvant according to any preceding claim, wherein the adjuvant is capable of forming particles at the site of radiation or inflammation.

13. The adjuvant according to any preceding claim, wherein the polymer comprises or consists of monomers of any of the group selected from N-isopropylacrylamide (NIPAM); N-isopropylmethacrylamide (NIPMAM); N,N′-diethylacrylamide (DEAAM); N-(L)-(1-hydroxymethyl) propyl methacrylamide (HMPMA); N,N′-dimethylethylmethacrylate (DMEMA), 2-(2-methoxyethoxy)ethyl methacrylate (DEGMA); pluronic, PLGA and poly(caprolactone); or combinations thereof.

14. The adjuvant according to any preceding claim, wherein the polymer comprises or consists of block-copolymer or graft-copolymer.

15. The adjuvant according to any preceding claim, wherein the Pattern Recognition Receptor (PRR) agonist comprises a PAMP (pathogen-associated molecular pattern).

16. The adjuvant according to any preceding claim, wherein the PRR agonist comprises a TLR agonist, a NOD-like receptor (NLR) agonist, C-type lectin receptor (CLRs), or agonists of STING.

17. The adjuvant according to any preceding claim, further comprising an antigen linked to the polymer or co-formulated with the polymer.

18. The adjuvant according to claim 17, wherein the antigen comprises a pathogen-derived antigen.

19. The adjuvant according to any of claim 17, wherein antigen comprises a cancer/tumour associated antigen or a tumor neoantigen that is specific to individual patients.

20. The adjuvant according to any preceding claim, wherein the PRR agonist molecules and/or antigens are linked to the monomer units distributed along the polymer backbone at a density of between about 1 mol % and about 100 mol %

21. The adjuvant according to any preceding claim, wherein the PRR agonist molecules and/or antigens are linked to the monomer units distributed along the polymer backbone at a density of between about 5 mol % and about 20 mol %.

22. The adjuvant according to any of claims 1 to 19, wherein the PRR agonist molecules and/or antigens are linked to the monomer units at or substantially near one end of the polymer (semi-telechelic); or linked to the monomer units at or substantially near both ends of the polymer (telechelic).

23. The adjuvant according to any preceding claim, wherein the PRR agonist and/or antigen are covalently linked to the polymer.

24. The adjuvant according to any preceding claim, wherein the PRR agonist and/or antigen are linked to the polymer by a linker molecule.

25. The adjuvant according to claim 24, wherein the linker is hydrophilic.

26. An immunogenic composition comprising an adjuvant and an antigen according to any preceding claim.

27. A method of treatment or prevention of a disease comprising the administration of an adjuvant or immunogenic composition according to any preceding claim to a subject in need thereof.

28. The adjuvant or immunogenic composition according to any of claims 1 to 25 for use as a medicament.

29. A method of eliciting an immune response for a disease comprising the administration of an adjuvant or immunogenic composition according to any of claims 1 to 26 to a subject in need thereof.

30. The method of eliciting an immune response according to claim 26, further comprising concurrent administration with an antigen, optionally wherein the concurrent administration is co-administration.

31. The method according to claim 29 or claim 30, wherein the method comprises the step of forming particles of the adjuvant in the subject by the action of a temperature shift from the administered adjuvant or immunogenic composition moving from outside the body to inside the body of the subject.

32. The method according to any of claims 29 to 31, further comprising the administration in combination with another active agent, such as a therapeutic molecule, biologic or different antigen.

33. A method of preparing an adjuvant comprising polymer particles, the method comprising the steps of:
providing an adjuvant or immunogenic composition according to any of claims 1 to 25;
filter sterilising the adjuvant or immunogenic composition; and
forming adjuvant particles by providing a temperature shift from below the lower critical solution temperature of the polymer to above the lower critical solution temperature of the polymer.

34. An adjuvant, immunogenic composition, vaccine, use or method substantially described herein, and optionally with reference to the accompanying figures.


SEQUENCE LISTING (4 sequences)

SEQ ID NO: 1 - protein, 13 aa, Homo sapiens
VLPDVFIRCVGGG

SEQ ID NO: 2 - protein, 10 aa, Homo sapiens
VLPDVFIRCV

SEQ ID NO: 3 - protein, 28 aa, Homo sapiens
  DOMAIN 10..18: note = CD8 EPITOPE
GRVLELFRAAQLANDVVLQIMELCGATR

SEQ ID NO: 4 - protein, 415 aa, Homo sapiens
GARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMGGGGGGGGGGIAALKSKIAALKSEIAALKSKIAALKSK